(19) United States
(12) Patent Application Publication
(10) Pub. No.: US 2023/0183249 A1
(43) Pub. Date: Jun. 15, 2023

(54) NEW TRIAZINOINDOLE COMPOUNDS

(71) Applicant: JANSSEN PHARMACEUTICA NV, Beerse (BE)

(72) Inventors: Daniel OEHLRICH, Beerse (BE); Michiel Luc Maria VAN GOOL, Madrid (ES); Juan Antonio VEGA RAMIRO, Madrid (ES); Mohamed LAMKANFI, Beerse (BE); Nina VAN OPDENBOSCH, Beerse (BE)

(21) Appl. No.: 17/997,135

(22) PCT Filed: Apr. 29, 2021
(86) PCT No.: PCT/EP2021/061260

§ 371 Date: Oct. 26, 2022

(30) Foreign Application Priority Data
Apr. 30, 2020 (EP) 20382360.4

(51) Int. Cl. C07D 487/04; C07D 519/00; A61K 45/06

(52) U.S. Cl. CPC C07D 487/04; C07D 519/00; A61K 45/06

(57) ABSTRACT

The invention relates to novel compounds for use as inhibitors of NLRP3 inflammasome production, wherein such compounds are as defined by compounds of formula (I) and wherein the integers R¹, R², R^{3a} and R^{3b} are defined in the description, and where the compounds may be useful as medicaments, for instance for use in the treatment of a disease or disorder that is associated with NLRP3 inflammasome activity.

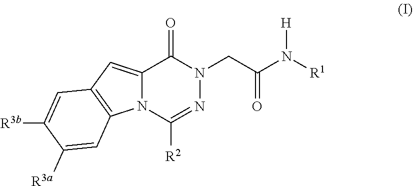

### FIELD OF THE INVENTION

The present invention relates to novel triazinones that are useful as inhibitors of NOD-like receptor protein 3 (NLRP3) inflammasome pathway. The present invention also relates to processes for the preparation of said compounds, pharmaceutical compositions comprising said compounds, methods of using said compounds in the treatment of various diseases and disorders, and medicaments containing them, and their use in diseases and disorders mediated by NLRP3.

### BACKGROUND OF THE INVENTION

Inflammasomes, considered as central signalling hubs of the innate immune system, are multi-protein complexes that are assembled upon activation of a specific set of intracellular pattern recognition receptors (PRRs) by a wide variety of pathogen- or danger-associated molecular patterns (PAMPs or DAMPs). To date, it was shown that inflammasomes can be formed by nucleotide-binding oligomerization domain (NOD)-like receptors (NLRs) and Pyrin- and HIN200-domain-containing proteins (Van Opdenbosch N and Lamkanfi M. Immunity, 2019 Jun. 18; 50(6):1352-1364). The NLRP3 inflammasome is assembled upon detection of environmental crystals, pollutants, host-derived DAMPs and protein aggregates (Tartey S and Kanneganti T D. Immunology, 2019 April; 156(4):329-338). Clinically relevant DAMPs that engage NLRP3 include uric acid and cholesterol crystals that cause gout and atherosclerosis, amyloid-β fibrils that are neurotoxic in Alzheimer's disease and asbestos particles that cause mesothelioma (Kelley et al., Int J Mol Sci, 2019 Jul. 6; 20(13)). Additionally, NLRP3 is activated by infectious agents such as Vibrio cholerae; fungal pathogens such as Aspergillus fumigatus and Candida albicans; adenoviruses, influenza A virus and SARS-CoV-2 (Tartey and Kanneganti, 2019 (see above); Fung et al. Emerg Microbes Infect, 2020 Mar. 14; 9(1):558-570).

Although the precise NLRP3 activation mechanism remains unclear, for human monocytes, it has been suggested that a one-step activation is sufficient while in mice a two-step mechanism is in place. Given the multitude in triggers, the NLRP3 inflammasome requires add-on regulation at both transcriptional and post-transcriptional level (Yang Y et al., Cell Death Dis, 2019 Feb. 12; 10(2):128).

The NLRP3 protein consists of an N-terminal pyrin domain, followed by a nucleotide-binding site domain (NBD) and a leucine-rich repeat (LRR) motif on C-terminal end (Sharif et al., Nature, 2019 June; 570(7761):338-343). Upon recognition of PAMP or DAMP, NLRP3 aggregates with the adaptor protein, apoptosis-associated speck-like protein (ASC), and with the protease caspase-1 to form a functional inflammasome. Upon activation, procaspase-1 undergoes autoproteolysis and consequently cleaves gasdermin D (Gsdmd) to produce the N-terminal Gsdmd molecule that will ultimately lead to pore-formation in the plasma membrane and a lytic form of cell death called pyroptosis. Alternatively, caspase-1 cleaves the pro-inflammatory cytokines pro-IL-1β and pro-IL-18 to allow release of its biological active form by pyroptosis (Kelley et al., 2019—see above).

Dysregulation of the NLRP3 inflammasome or its downstream mediators are associated with numerous pathologies ranging from immune/inflammatory diseases, auto-immune/auto-inflammatory diseases (Cryopyrin-associated Periodic Syndrome (Miyamae T. Paediatr Drugs, 2012 Apr. 1; 14(2):109-17); sickle cell disease; systemic lupus erythematosus (SLE)) to hepatic disorders (eg. non-alcoholic steatohepatitis (NASH), chronic liver disease, viral hepatitis, alcoholic steatohepatitis, and alcoholic liver disease) (Szabo G and Petrasek J. Nat Rev Gastroenterol Hepatol, 2015 July; 12(7):387-400) and inflammatory bowel diseases (eg. Crohn's disease, ulcerative colitis) (Zhen Y and Zhang H. Front Immunol, 2019 Feb. 28; 10:276). Also, inflammatory joint disorders (eg. gout, pseudogout (chondrocalcinosis), arthropathy, osteoarthritis, and rheumatoid arthritis (Vande Walle L et al., Nature, 2014 Aug. 7; 512(7512):69-73) were linked to NLRP3. Additionally, kidney related diseases (hyperoxaluria (Knauf et al., Kidney Int, 2013 November; 84(5):895-901), lupus nephritis, hypertensive nephropathy (Krishnan et al., Br J Pharmacol, 2016 February; 173(4):752-65), hemodialysis related inflammation and diabetic nephropathy which is a kidney-related complication of diabetes (Type 1, Type 2 and mellitus diabetes), also called diabetic kidney disease (Shahzad et al., Kidney Int, 2015 January; 87(1):74-84) are associated to NLRP3 inflammasome activation. Reports link onset and progression of neuroinflammation-related disorders (eg. brain infection, acute injury, multiple sclerosis, Alzheimer's disease) and neurodegenerative diseases (Parkinsons disease) to NLRP3 inflammasome activation (Sarkar et al., NPJ Parkinsons Dis, 2017 Oct. 17; 3:30). In addition, cardiovascular or metabolic disorders (eg. cardiovascular risk reduction (CvRR), atherosclerosis, type I and type II diabetes and related complications (e.g. nephropathy, retinopathy), peripheral artery disease (PAD), acute heart failure and hypertension (Ridker et al., CANTOS Trial Group. N Engl J Med 2017 Sep. 21; 377(12):1119-1131; and Toldo S and Abbate A. Nat Rev Cardiol, 2018 April, 15(4):203-214) have recently been associated to NLRP3. Also, skin associated diseases were described (eg. wound healing and scar formation; inflammatory skin diseases, eg. acne, hidradenitis suppurativa (Kelly et al., Br J Dermatol, 2015 December; 173(6)). In addition, respiratory conditions have been associated with NLRP3 inflammasome activity (eg. asthma, sarcoidosis, Severe Acute Respiratory Syndrome (SARS) (Nieto-Torres et al., Virology, 2015 November; 485:330-9)) but also age-related macular degeneration (Doyle et al., Nat Med, 2012 May; 18(5):791-8). Several cancer related diseases/disorders were described linked to NLRP3 (eg. myeloproliferative neoplasms, leukemias, myelodysplastic syndromes (MOS), myelofibrosis, lung cancer, colon cancer (Ridker et al., Lancet, 2017 Oct. 21; 390(10105):1833-1842; Derangere et al., Cell Death Differ. 2014 December; 21(12):1914-24; Basiorka et al., Lancet Haematol, 2018 September; 5(9): e393-e402, Zhang et al., Hum Immunol, 2018 January; 79(1):57-62).

Several patent applications describe NLRP3 inhibitors, with recent ones including for instance international patent application WO 2020/018975, WO 2020/037116, WO 2020/021447, WO 2020/010143, WO 2019/079119, WO 2019/0166621, WO 2019/121691 and WO 2019/209896, which disclose a range of specific compounds. Various specific compounds can be identified through the Chemical Abstracts Service, for instance compounds that have no ascribed use.

There is a need for inhibitors of the NLRP3 inflammasome pathway to provide new and/or alternative treatments for the diseases/disorders mentioned herein.

### SUMMARY OF THE INVENTION

The invention provides compounds which inhibit the NLRP3 inflammasome pathway.

Thus, in an aspect of the invention, there is now provided a compound of formula (I),

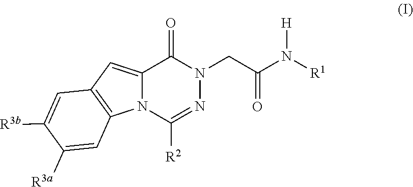

or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
   R² represents:
(i) hydrogen;
(ii) halo;
(iii) —CN;
(iv) C₁₋₆ alkyl optionally substituted with one or more substituents independently selected from halo, —OH, —OC₁₋₃alkyl and oxo;
(v) C₃₋₆ cycloalkyl;
(vi) C₂₋₄alkenyl optionally substituted with —OC₁₋₃alkyl;
(vii) —O—C₁₋₃alkyl;
(viii) —N(R^{2a})R^{2b}; or
(ix) 5-membered heteroaryl, optionally substituted by one or more substituents selected from halo, C₁₋₃ alkyl and —OC₁₋₃ alkyl;
   each R^{2a} and R^{2b} independently represent hydrogen or C₁₋₄alkyl optionally substituted with —OC₁₋₃ alkyl;
   either one of R^{3a} and R^{3b} represents hydrogen and the other represents R³;
   R³ represents:
(i) hydrogen;
(ii) halo; or
(iii) C₁₋₃ alkyl (e.g. methyl),
   which compounds may referred to herein as “compounds of the invention”.

In an embodiment, compounds of the invention that may be mentioned include those in which:
(i) when R² represents hydrogen, R^{3a} and R^{3b} both represent hydrogen, then R¹ does not represent 2,3,4-trimethoxyphenyl, 2,4-dimethylcyclohexyl, 2-ethylphenyl, 3,4-dimethoxyphenyl, 3,4-dimethylphenyl, 3,5-dimethylphenyl, 3-ethylphenyl, 3-fluorophenyl, 4-ethylphenyl, 4-isopropylphenyl (or 4-propan-2-yl-phenyl) or cyclopropyl;
(ii) when R² represents hydrogen, R^{3a} represents hydrogen and R^{3b} represents fluoro, then R¹ does not represent 1,2,3,4-tetrahydronaphthalen-1-yl, 1(R)-1,2,3,4-tetrahydronaphthalen-1-yl, cyclohexyl or cyclopropyl;
(iii) when R² represents methyl, R^{3a} represents hydrogen and R represents fluoro, then R¹ does not represent 1(S),2(R)-2-methylcyclohexyl, 2-methylcyclohexyl, 2,3-dimethylcyclohexyl, (1R),(2R),3(R)-2,3-dimethylcyclohexyl, (1R),(2R),3(S)-2,3-dimethylcyclohexyl, (1R),(2S),3(R)-2,3-dimethylcyclohexyl, (1R),(2S),3(S)-2,3-dimethylcyclohexyl, cyclohexyl or cyclopropyl;
(iv) when R² represents methyl or ethyl, R^{3a} and R^{3b} both represent hydrogen, then R¹ does not represent cyclopropyl,
   which we may refer to herein as “the provisos”.

Hence, there is provided a compound of formula (I) as hereinbefore defined, or a pharmaceutically acceptable salt thereof, provided that it is not a compound of the provisos.

In an aspect of the invention, there is provided a compound of formula (I) as hereinbefore defined, or a pharmaceutically acceptable salt thereof, R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
   R² represents:
(i) hydrogen;
(ii) halo;
(iii) —CN;
(iv) C₁₋₆ alkyl optionally substituted with one or more substituents independently selected from halo, —OH, —OC₁₋₃alkyl and oxo;
(v) C₃₋₆ cycloalkyl;
(vi) C₂₋₄alkenyl optionally substituted with —OC₁₋₃alkyl;
(vii) —O—C₁₋₃alkyl;
(viii) —N(R^{2a})R^{2b}; or
(ix) 5-membered heteroaryl, optionally substituted by one or more substituents selected from halo, C₁₋₃ alkyl and —OC₁₋₃ alkyl;
each R^{2a} and R^{2b} independently represent hydrogen or C₁₋₄alkyl optionally substituted with —OC₁₋₃ alkyl;
   either one of R^{3a} and R^{3b} represents hydrogen and the other represents R³;
   R³ represents:
(i) hydrogen; or
(ii) halo,
   provided that:
(i) when R² represents hydrogen, R^{3a} and R^{3b} both represent hydrogen, then R¹ does not represent 2,3,4-trimethoxyphenyl, 2,4-dimethylcyclohexyl, 2-ethylphenyl, 3,4-dimethoxyphenyl, 3,4-dimethylphenyl, 3,5-dimethylphenyl, 3-ethylphenyl, 3-fluorophenyl, 4-ethylphenyl, 4-isopropylphenyl (or 4-propan-2-yl-phenyl) or cyclopropyl;
(ii) when R² represents hydrogen, R^{3a} represents hydrogen and R^{3b} represents fluoro, then R¹ does not represent 1,2,3,4-tetrahydronaphthalen-1-yl, 1(R)-1,2,3,4-tetrahydronaphthalen-1-yl, cyclohexyl or cyclopropyl;
(iii) when R² represents methyl, R^{3a} represents hydrogen and R^{3b} represents fluoro, then R¹ does not represent I(S),2(R)-2-methylcyclohexyl, 2-methylcyclohexyl, 2,3-dimethylcyclohexyl, (1R),(2R),3(R)-2,3-dimethylcyclohexyl, (1R),(2R),3(S)-2,3-dimethylcyclohexyl, (1R),(2S),3(R)-2,3-dimethylcyclohexyl, (1R),(2S),3(S)-2,3-dimethylcyclohexyl, cyclohexyl or cyclopropyl;
(iv) when R² represents methyl or ethyl, R^{3a} and R^{3b} both represent hydrogen, then R¹ does not represent cyclopropyl,
   which compounds may also be referred to herein as “compounds of the invention”.

In another aspect, there is provided compounds of the invention (without the provisos, where applicable) for use as a medicament. In another aspect, there is provided a pharmaceutical composition comprising a therapeutically effective amount of a compound of the invention (without the provisos, again where applicable).

In a further aspect, there is provided compounds of the invention (without the provisos) (and/or pharmaceutical compositions comprising such compounds) for use: in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor. Specific diseases or disorders may be mentioned herein, and may for instance be selected from inflammasome-related diseases or disorders, immune diseases, inflammatory diseases, auto-immune diseases, or auto-inflammatory diseases.

In another aspect, there is provided a use of compounds of the invention (without the provisos) (and/or pharmaceutical compositions comprising such compounds): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In another aspect, there is provided use of compounds of the invention (without the provisos) (and/or pharmaceutical compositions comprising such compounds) in the manufacture of a medicament for: the treatment of a disease or disorder associated with NLRP3 activity (including inflammasome activity); the treatment of a disease or disorder in which the NLRP3 signalling contributes to the pathology, and/or symptoms, and/or progression, of said disease/disorder; and/or inhibiting NLRP3 inflammasome activity (including in a subject in need thereof).

In another aspect, there is provided a method of treating a disease or disorder in which the NLRP3 signalling contributes to the pathology, and/or symptoms, and/or progression, of said disease/disorder, comprising administering a therapeutically effective amount of a compound of the invention (without the provisos), for instance to a subject (in need thereof). In a further aspect there is provided a method of inhibiting the NLRP3 inflammasome activity in a subject (in need thereof), the method comprising administering to the subject in need thereof a therapeutically effective amount of a compound of the invention (without the provisos).

In further aspect, there is a provided a compound of the invention (without the provisos) in combination (including a pharmaceutical combination) with one or more therapeutic agents (for instance as described herein). Such combination may also be provided for use as described herein in respect of compounds of the invention, or, a use of such combination as described herein in respect of compounds of the invention. There may also be provided methods as described herein in respect of compounds of the invention, but wherein the method comprises administering a therapeutically effective amount of such combination.

### DETAILED DESCRIPTION OF THE INVENTION

The invention provides a compound of formula (I),

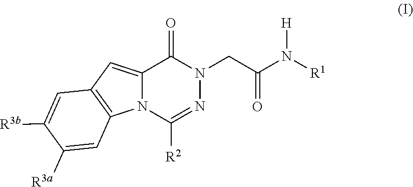

or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
   R² represents:
(i) hydrogen;
(ii) halo;
(iii) —CN;
(iv) C₁₋₆ alkyl optionally substituted with one or more substituents independently selected from halo, —OH, —OC₁₋₃alkyl and oxo;
(v) C₃₋₆ cycloalkyl;
(vi) C₂₋₄alkenyl optionally substituted with —OC₁₋₃alkyl;
(vii) —O—C₁₋₃alkyl;
(viii) —N(R^{2a})R^{2b}; or
(ix) 5-membered heteroaryl, optionally substituted by one or more substituents selected from halo, C₁₋₃ alkyl and —OC₁₋₃ alkyl;
   each R² and R^{2b} independently represent hydrogen or C₁₋₄alkyl optionally substituted with —OC₁₋₃ alkyl;
   either one of R^{3a} and R^{3b} represents hydrogen and the other represents R³;
   R³ represents:
(i) hydrogen;
(ii) halo; or
(iii) C₁₋₃ alkyl (e.g. methyl).

As indicated above, such compounds may be referred to herein as “compounds of the invention”.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine

For the purposes of this invention solvates, prodrugs, N-oxides and steroisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

When an absolute configuration is specified, it is according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain. Such a group is attached to the rest of the molecule by a single bond.

C_{2-q} alkenyl when used herein (again where q is the upper limit of the range) refers to an alkyl group that contains unsaturation, i.e. at least one double bond.

C_{3-q} cycloalkyl (where q is the upper limit of the range) refers to an alkyl group that is cyclic, for instance cycloalkyl groups may be monocyclic or, if there are sufficient atoms, bicyclic. In an embodiment, such cycloalkyl groups are monocyclic.

Such cycloalkyl groups are unsaturated. Substituents may be attached at any point on the cycloalkyl group.

The term “halo”, when used herein, preferably includes fluoro, chloro, bromo and iodo.

C_{1-q} alkoxy groups (where q is the upper limit of the range) refers to the radical of formula —OR^{a}, where R^{a} is a C^{1-q} alkyl group as defined herein.

HaloC_{1-q} alkyl (where q is the upper limit of the range) groups refer to C_{1-q} alkyl groups, as defined herein, where such group is substituted by one or more halo. HydroxyC_{1-q} alkyl (where q is the upper limit of the range) refers to C_{1-q} alkyl groups, as defined herein, where such group is substituted by one or more (e.g. one) hydroxy (—OH) groups (or one or more, e.g. one, of the hydrogen atoms is replaced with —OH). Similarly, haloC_{1-q} alkoxy and hydroxyC_{1-q} alkoxy represent corresponding —OC_{1-q} alkyl groups that are substituted by one or more halo, or, substituted by one or more (e.g. one) hydroxy, respectively.

Heterocyclyl groups that may be mentioned include non-aromatic monocyclic and bicyclic heterocyclyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocyclyl groups may also be bridged. Such heterocyclyl groups are saturated. C_{2-q} heterocyclyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocyclyl groups may, where appropriate, be located on any atom in the ring system including a heteroatom. The point of attachment of heterocyclyl groups may be via any atom in the ring system including (where appropriate) a heteroatom (such as a nitrogen atom), or an atom on any fused carbocyclic ring that may be present as part of the ring system. Heterocyclyl groups may also be in the N- or S-oxidised form. In an embodiment, heterocyclyl groups mentioned herein are monocyclic.

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. When aryl groups are polycyclic, in an embodiment, each ring is aromatic. In an embodiment, aryl groups mentioned herein are monocyclic or bicyclic. In a further embodiment, aryl groups mentioned herein are monocyclic.

“Heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. In an embodiment, when heteroaryl groups are polycyclic, then each ring is aromatic. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. When heteroaryl groups are polycyclic in which there is a non-aromatic ring present, then that non-aromatic ring may be substituted by one or more ═O group. In an embodiment, heteroaryl groups mentioned herein may be monocyclic or bicyclic. In a further embodiment, heteroaryl groups mentioned herein are monocyclic.

Heteroatoms that may be mentioned include phosphorus, silicon, boron and, preferably, oxygen, nitrogen and sulfur.

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

Various embodiments of the invention will now be described, including embodiments of the compounds of the invention.

In an embodiment, there is provided a compound of formula (I), as hereinbefore defined, or a pharmaceutically acceptable salt thereof, wherein: R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
   R² represents:
(i) hydrogen;
(ii) halo;
(iii) —CN;
(iv) C₁₋₆ alkyl optionally substituted with one or more substituents independently selected from halo, —OH, —OC₁₋₃alkyl and oxo;
(v) C₃₋₆ cycloalkyl;
(vi) C₂₋₄alkenyl optionally substituted with —OC₁₋₃alkyl;
(vii) —O—C₁₋₃alkyl;
(viii) —N(R^{2a})R^{2b}; or
(ix) 5-membered heteroaryl, optionally substituted by one or more substituents selected from halo, C₁₋₃ alkyl and —OC₁₋₃ alkyl;
   each R^{2a} and R^{2b} independently represent hydrogen or C₁₋₄alkyl optionally substituted with —OC₁₋₃ alkyl;
   either one of R^{3a} and R^{3b} represents hydrogen and the other represents R³;
   R³ represents:
(i) hydrogen; or
(ii) halo.

In an embodiment, there is provided compounds of the invention in which:
at least one of R^{3a} and R^{3b} does not represent hydrogen;
R² does not represent hydrogen;
R² does not represent methyl;
when R² represents ethyl, then R¹ does not represent cycloalkyl (hence R¹ represents, in this instance, aryl or heteroaryl or heterocyclyl, all of which are optionally substituted as herein defined; and/or
R² does not represent ethyl.

In another embodiment, there is provided compounds of the invention in which:

R² represents:
(i) hydrogen;
(ii) halo;
(iii) —CN;
(iv) C₃₋₆ alkyl optionally substituted with one or more substituents independently selected from halo, —OH, —OC₁₋₃alkyl and oxo;
(v) C₃₋₆ cycloalkyl;
(vi) C₂₋₄alkenyl optionally substituted with —OC₁₋₃alkyl;
(vii) —O—C₁₋₃alkyl;
(viii) —N(R^{2a})R^{2b}; or
(ix) 5-membered heteroaryl, optionally substituted by one or more substituents selected from halo, C₁₋₃ alkyl and —OC₁₋₃ alkyl.

In an embodiment, compounds of the invention include those in which R¹ represents: (i) C₃₋₆ cycloalkyl; (ii) aryl or heteroaryl; or (iii) or heterocyclyl, all of which are optionally substituted as herein defined.

In an embodiment when R¹ represents optionally substituted C₃₋₆ cycloalkyl, then it represents C₃₋₆ cycloalkyl (or, in an embodiment, C₃₋₄ cycloalkyl) optionally substituted by one or two substituents selected from C₁₋₃ alkyl (e.g. methyl) and —OH. In a further embodiment, R¹ represents cyclopropyl (e.g. unsubstituted) or cyclobutyl. In a further embodiment, R¹ represents cyclohexyl. In yet a further embodiment, R¹ represents unsubstituted cyclopropyl or cyclobutyl substituted by —OH and methyl (e.g. at the same carbon atom). In yet a further embodiment, R¹ represents cyclohexyl, for instance substituted by —OH (e.g. by one —OH group). In an embodiment therefore, R¹ represents:

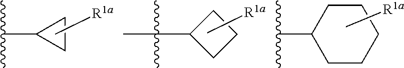

where each R^{1a} represents one or two optional substituents selected from —OH and C₁₋₃ alkyl (e.g. methyl). In a particular embodiment of this aspect, R¹ represents C₃₋₆ cycloalkyl, such as optionally substituted cyclohexyl, optionally substituted cyclobutyl or unsubstituted (or optionally substituted) cyclopropyl, for instance:

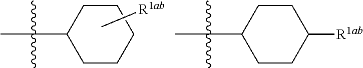

where each R^{1ab} represents one or two optional substituents selected from those defined by R^{1a}, and in an embodiment, represents one optional substituent selected from —OH;

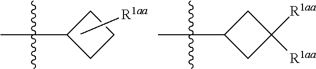

where each R^{1aa} represents one or two optional substituents selected from those defined by R^{1a}, and in an embodiment represents two substituents, methyl and —OH; or

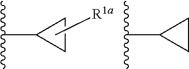

where R^{1a} is as defined above, but where, in a particular embodiment, it is not present.

In an embodiment where R¹ represents aryl or heteroaryl, optionally substituted as defined herein, then it may represent: (i) phenyl; (ii) a 5- or 6-membered mono-cyclic heteroaryl group; or (iii) a 9- or 10-membered bicyclic heteroaryl group, all of which are optionally substituted by one to three substituents as defined herein. In an embodiment, the aforementioned aryl and heteroaryl groups are optionally substituted with one or two (e.g. one) substituent(s) selected from halo (e.g. fluoro), —OH, C₁₋₃ alkyl and —OC₁₋₃ alkyl. In a further embodiment, such optional substituents are selected from fluoro, methyl, hydroxy and methoxy. In one embodiment, R¹ represents phenyl or a mono-cyclic 5- or 6-membered heteroaryl group (in one aspect a 5-membered heteroaryl group) and in another embodiment it may represent a 9- or 10-membered (e.g. 9-membered) bicyclic heteroaryl group. Hence, in an embodiment, R¹ may represent:

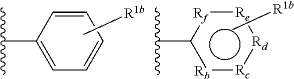

wherein R^{1b} represents one or two optional substituents selected from halo, —CH₃, —OH and —OCH₃ (and in a further embodiment, such optional substituents are selected from fluoro and methoxy), and at least one of R_{b}, R_{c}, R_{d}, Rₑ and R_{f} represents a nitrogen heteroatom (and the others represent CH). In an embodiment, either one or two of R_{b}, R_{c}, R_{d}, Rₑ and R_{f} represent(s) a nitrogen heteroatom, for instance, R_{d} represents nitrogen and, optionally, R_{b} represents nitrogen, or, R_{c} represents nitrogen. In an aspect: (i) R_{b} and R_{d} represent nitrogen; (ii) R_{d} represents nitrogen; or (iii) R_{c} represents nitrogen. Hence, R¹ may represent 3-pyridyl, 4-pyridyl or 4-pyrimidinyl, all of which are optionally substituted as herein defined, for instance with one substituent selected from fluoro, methyl, hydroxy and methoxy (for instance, selected from methyl, —OH and —OCH₃). In a further embodiment, R¹ represents unsubstituted phenyl, 2-methoxyphenyl, unsubstituted 4-pyrimidinyl, unsubstituted 4-pyridyl, unsubstituted 3-pyridyl, 3-fluoro-4-pyridyl, 3-methoxy-4-pyridyl, 2-methoxy-3-pyridyl or 2-methyl-4-pyridyl. In another embodiment, R¹ may represent:

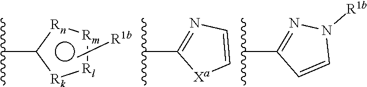

wherein R^{1b} is as defined above (i.e. represents one or two optional substituents) but in an aspect, is preferably not present (and, as such, in an embodiment, represents an unsubstituted 5-membered heteroaryl group), and at least one of Rₖ, Rₗ, Rₘ and Rₙ represents a heteroatom, and in an embodiment, at least one of these represents N and the others are independently selected from CH, N, O and S (provided that the rules of valency are adhered to); for instance, in an embodiment, one of Rₖ and Rₙ represents N, the other represents N, O, S or CH, and Rₗ and Rₘ each represent CH, and, in a further particular embodiment, X^{a} represents N, O, S or CH, for instance X^{a} represents O, so forming a 2-oxazolyl group. As such, in a particular embodiment, R¹ represents unsubstituted 2-oxazolyl. In another particular embodiment, R¹ represents a 3-pyrazolyl group (for instance in which Rₖ and Rₗ represents N, Rₙ and Rₘ represent CH, and R^{1b} represents a C₄ alkyl (e.g. isopropyl) that is on the 1-(N) atom). In another embodiment, R¹ may represent:

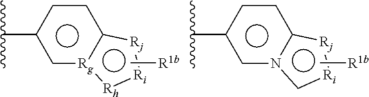

wherein R^{1b} is as defined above (i.e. represents one or two optional substituent as defined above), each ring of the bicyclic system is aromatic, R_{g} represents a N or C atom and any one or two of Rₕ, Rᵢ and Rⱼ (for instance, one or two of Rᵢ and Rⱼ) represents N and the other(s) represent(s) C (provided that, as the skilled person would understand, the rules of valency are adhered to; for instance when one of the atoms of the (hetero)aromatic ring represents C, then it is understood that it may bear a H atom).

In an embodiment R¹ represents:

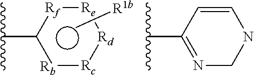

in which R_{b} and R_{d} represent a nitrogen atom, and, in an embodiment, there is no R^{1b} substituent present.

In another embodiment, R¹ represents:

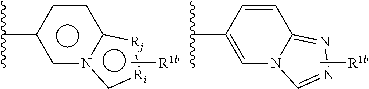

in which one of Rᵢ and Rⱼ represents N and the other represents C, or, both Rᵢ and Rⱼ represent N, and, in an embodiment, there is no R^{1b} substituent present.

In a further embodiment, R¹ represents phenyl or a 6-membered heteroaryl group (containing between one and three heteroatoms) and which is optionally substituted as defined herein. In an embodiment, R¹ represents a 6,5-fused bicyclic ring containing one to five heteroatoms (wherein at least two are nitrogen) and which group is optionally substituted as herein defined.

In a further embodiment, R¹ represents:

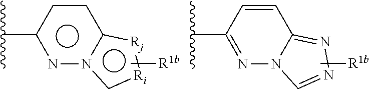

in which Rⁱ, R^{j} and R^{1b} are as hereinbefore defined.

In an embodiment where R¹ represents heterocyclyl, optionally substituted as defined herein, such group is in a further aspect a 5- or 6-membered heterocyclyl group, for instance containing at least one nitrogen or oxygen heteroatom; for instance, in a particular embodiment, in this instance R¹ may represent a 6-membered nitrogen-containing heterocyclyl group optionally substituted by one substituent selected from C₁₋₃ alkyl and C₃₋₆ cycloalkyl. In an aspect of this embodiment, the 6-membered heterocyclyl group may be piperidinyl (e.g. 3-piperidinyl) optionally substituted by C₃₋₄ cycloalkyl (e.g. cyclobutyl) or the 6-membered heterocyclyl group may be tetrahydropyran, e.g. 4-tetrahydropyranyl (which is preferably unsubstituted).

In an embodiment where R¹ represents aryl, specific groups that may be mentioned include phenyl and methoxy-phenyl (such as 2-methoxy-phenyl). In an embodiment where R¹ represents heteroaryl, it is a mono-cyclic 6-membered ring, for instance containing at least one nitrogen heteroatom and thereby forming a pyridyl or pyrimidinyl group, or, it is a mono-cyclic 5-membered ring, for instance containing at least one nitrogen heteroatom, so forming e.g. an oxazolyl (e.g. 2-oxazolyl) group. Specific mono-cyclic heteroaryl groups that R¹ may represent include 4-pyridyl, 3-pyridy, 4-pyrimidinyl and 2-oxazolyl (all of which are optionally substituted as defined herein). In view of the optional substitution mentioned herein, such groups may represent an unsubstituted 4-pyrimidinyl, unsubstituted 3-pyridyl, 2-methoxy-3-pyridyl, 2-methyl-4-pyridyl and unsubstituted 2-oxazolyl group.

In a particular embodiment, R¹ represents cyclopropyl or a mono-cyclic heteraryl group optionally substituted as defined herein. In an aspect, R¹ represents a mono-cyclic heteroaryl group, for instance a 6-membered mono-cyclic heteroaryl group containing one or two nitrogen heteroatoms, and which groups is optionally substituted by one or more substituents selected from fluoro and methoxy.

In an embodiment R² represents: (i) hydrogen; (ii) halo; (iii) —CN; (iv)C₁₋₄ alkyl optionally substituted with one or more substituents independently selected from halo, —OH and —OC₁₋₂ alkyl; (v) Cm cycloalkyl; (vi) —O—C₁₋₂alkyl; (vii) —N(R^{2a})R^{2b}; or (viii) 5-membered heteroaryl.

In an embodiment when R² represents optionally substituted C₁₋₆ alkyl, then it represents C₁₋₄ alkyl optionally substituted by one or more substitutents selected from fluoro and —OH. In an embodiment when R² represents C₃₋₆ cycloalkyl, then it represents unsubstituted C₃₋₆ cycloalkyl. In an embodiment when R² represents —OC₁₋₃ alkyl, then it represents unsubstituted —OC¹⁻² alkyl. In an embodiment when R² represents —N(R^{2a})R^{2b}, then one of R^{2a} and R^{2b} represents C₁₋₃ alkyl and the other represents hydrogen or C₁₋₃ alkyl, or, for instance both R^{2a} and R^{2b} represent unsubstituted C₁₋₃ alkyl. In an embodiment when R² represents 5-membered heteroaryl, then it represents a 5-membered heteroaryl group containing one or two heteroatoms selected from nitrogen, oxygen and sulfur, and which heteroaryl group is preferably unsubstituted.

In an embodiment, each R^{2a} and R^{2b} independently represent hydrogen or unsubstituted C₁₋₄ alkyl, and, in an embodiment, R^{2a} and R^{2b} independently represent C₁₋₃ alkyl (such as methyl).

In an embodiment, specific R² groups that may be mentioned include hydrogen, chloro, —CN, methyl, ethyl, isopropyl, isobutyl (—CH₂C(H)(CH₃)₂), —CHF₂, —CF₃, —C(CH₃)F₂, —C(H)(CH₃)OH, cyclopropyl, cyclobutyl, cyclopentyl, cyclohexyl, —OCH₃, —N(CH₃)₂, thienyl (including 2-thienyl) and oxazolyl (including 2-oxazolyl). In an alternative embodiment, R² represents —N(C₁₋₃ alkyl)₂ or —OC₁₋₃alkyl (for instance, —N(CH₃)₂ or —OCH₃).

In an embodiment, R³ represents (i) hydrogen; or (ii) fluoro. Hence, in an aspect, one of R^{3a} and R^{3b} represents hydrogen and other represents hydrogen or fluoro. In an embodiment, both R^{3a} and R^{3b} represent hydrogen. In another embodiment, R^{3b} represents hydrogen and R^{3a} represents fluoro. In an alternative embodiment, one of R^{3a} and R^{3b} represents methyl and the other represents hydrogen.

The names of the compounds of the present invention were generated according to the nomenclature rules agreed upon by the Chemical Abstracts Service (CAS) using Advanced Chemical Development, Inc., software (ACD/Name product version 10.01; Build 15494, 1 Dec. 2006) or according to the nomenclature rules agreed upon by the International Union of Pure and Applied Chemistry (IUPAC) using Advanced Chemical Development, Inc., software (ACD/Name product version 10.01.0.14105, October 2006). In case of tautomeric forms, the name of the depicted tautomeric form of the structure was generated. The other non-depicted tautomeric form is also included within the scope of the present invention.

### Preparation of the Compounds

In an aspect of the invention, them is provided a process for the preparation of compounds of the invention, where reference here is made to compounds of formula (I) as defined herein.

Compounds of formula (I) may be prepared by:
(i) reaction of a compound of formula (II),

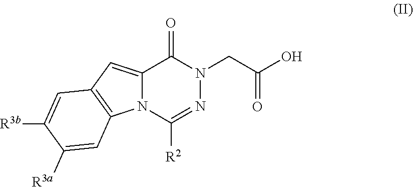

or a derivative thereof (e.g. a salt), wherein R², R^{3a} and R^{3b} are as hereinbefore defined, with a compound of formula (III),

H₂N—R¹ (III)

or a derivative thereof, wherein R¹ is as hereinbefore defined, under amide-forming reaction conditions (also referred to as amidation), for example in the presence of a suitable coupling reagent (e.g. propylphosphonic anhydride, I-[bis(dimethylamino)methylene]-1H-1,2,3-triazolo[4,5-b]pyridinium 3-oxide hexafluorophosphate (O-(7-azabenzotriazol-1-yl)-N,N,N′,N′-tetramethyluronium hexafluorophosphate), 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosponium hexafluorophosphate, 2-(1H-benzotriazol-1-yl)-1,1,3,3-tetramethyluronium tetra-fluorocarbonate, 1-cyclohexylcarbodiimide-3-propyloxymethyl polystyrene, O-benzotriazol-1-yl-N,N,N′,N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. Alternatively, a carboxylic acid group may be converted under standard conditions to the corresponding acyl chloride (e.g. in the presence of SOCl₂ or oxalyl chloride), which acyl chloride is then reacted with a compound of formula (II), for example under similar conditions to those mentioned above;

(ii) reaction of a compound of formula (IV),

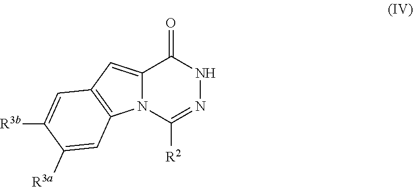

wherein R², R^{3a} and R^{3b} are as hereinbefore defined, with a compound of formula (V),

LG^{a}-CH₂—C(O)—N(H)R¹ (V)

wherein LG^{a} represents a suitable leaving group (e.g. halo, such as chloro) and R¹ is as defined herein, under suitable reaction conditions, e.g. in the presence of an appropriate base, e.g. Cs₂CO₃, K₂CO₃ or LiHMDS, or the like, or alternative alkylation reaction conditions;

(iii) by transformation (such transformation steps may also take place on intermediates) of a certain compound of formula (I) into another, for example:
for compounds of formula (I) in which R² represents —N(R^{2a})R^{2b}, reaction of a corresponding compound of formula (I) in which R² represents halo, with an appropriate amine HN(R^{2a})R^{2b} (wherein R^{2a} and R^{2b} are as herein defined), in an amination reaction under appropriate conditions, e.g. using under standard coupling conditions, in the presence of a catalyst, e.g. CuI, a ligand, e.g. D/L-proline and a base, e.g. K₂CO₃; similar transformations may be performed on compounds in which another group represents halo, and an amine is desired at another position;
for compounds of formula (I) containing an alkene, reduction to a corresponding compound of formula (I) containing an alkane, under reduction conditions, e.g. with hydrogen in the presence of a suitable catalyst such as, for example, palladium on carbon, in a suitable reaction-inert solvent, such as, for example, ethanol or methanol;
coupling to convert a halo or triflate group to e.g. an alkyl, alkenyl or aryl/heteroaryl group, for example in the presence of a suitable coupling reagent, e.g. where the reagent comprises the appropriate alkyl, alkenyl or aryl/heteroaryl group attached to a suitable group such as —B(OH)₂, —B(OR^{wx})₂, zincates (e.g. including —Zn(R^{wx})₂, —ZnBrR^{wx}) or —Sn(R^{wx})₃, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), thereby forming e.g. a pinacolato boronate ester group. The reaction may be performed in the presence of a suitable catalyst system, e.g. a metal (or a salt or complex thereof) such as Pd, CuI, Pd/C, PdCl2, Pd(OAc)₂, Pd(Ph₃P)₂Cl₂, Pd(Ph₃P)₄ (i.e. palladium tetrakistriphenylphosphine), Pd₂(dba)₃ and/or NiCl₂ (preferred cataysts include RuPhos Pd G3, XPhos Pd and bis(tri-tert-butylphosphine)palladium(0)) and optionally a ligand such as PdCl₂(dppf).DCM, t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-bi-naphthyl, 1,1′-bis(diphenyl-phosphino-ferrocene), 1,3-bis(diphenylphosphino)propane, xantphos, or a mixture thereof, together with a suitable base, such as Na₂CO₃, K₃PO₄, Cs₂CO₃, NaOH, KOH, K₂CO₃, CsF, Et₃N, (i-Pr)₂NEt, t-BuONa or t-BuOK (or mixtures thereof; preferred bases include Na₂CO₃ and K₂CO₃) in a suitable solvent such as dioxane, toluene, ethanol, dimethylformamide, dimethoxyethane, ethylene glycol dimethyl ether, water, dimethylsulfoxide, acetonitrile, dimethylacetamide, N-methylpyrrolidinone, tetrahydrofuran or mixtures thereof (preferred solvents include dimethylformamide and dimethoxyethane);
reduction of a ketone to an alcohol, in the presence of suitable reducing conditions, e.g. NaBH₄ or the like;
conversion of —C(═CH₂)—OCH₂CH₃ to —C(O)CH₃, by reaction in the presence of HCl, e.g. also in a suitable solvent such as THF;
conversion of a —C(O)alkyl moiety to a —C(OH)(alkyl)(alkyl) moiety by reaction of an appropriate Grignard reagent, e.g. alkylMgBr;
transformation of a alkene ═CH₂ moiety to a carbonyl ═O moiety, for instance, in the presence of AD-mix-Alpha and methane-sulfonamide;
transformation of a ketone to an alcohol —OH moiety;
alkylation of a —OH moiety (to —O-alkyl), under appropriate reaction conditions.

The compound of formula (II) may be prepared by hydrolysis of the corresponding carboxylic acid ester (for example under standard hydrolysis conditions, e.g. base hydrolysis in the presence of an alkali metal hydroxide (such as lithium hydroxide)), which in turn is prepared by reaction of a compound of formula (IV),

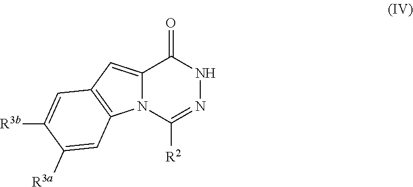

wherein R², R^{3a} and R^{3b} are as hereinbefore defined, with a compound of formula (VI),

LG-CH₂—C(O)O—R^{aa} (VI)

wherein R^{aa} represents C₁₋₆ alkyl (e.g ethyl) and LG represents a suitable leaving group, such as halo (e.g. chloro), for instance under reaction conditions and using reagent such as those described herein.

In general the compounds of the invention can therefore be made with reference to the procedures above. However, in the interests of versatility, further schemes are provided below in order to provide intermediate and final compounds of the invention. Further details are provided in the schemes below (as well as in the specific details of the experimental described hereinafter).

In this respect, Scheme 1 outlines a typical synthesis:

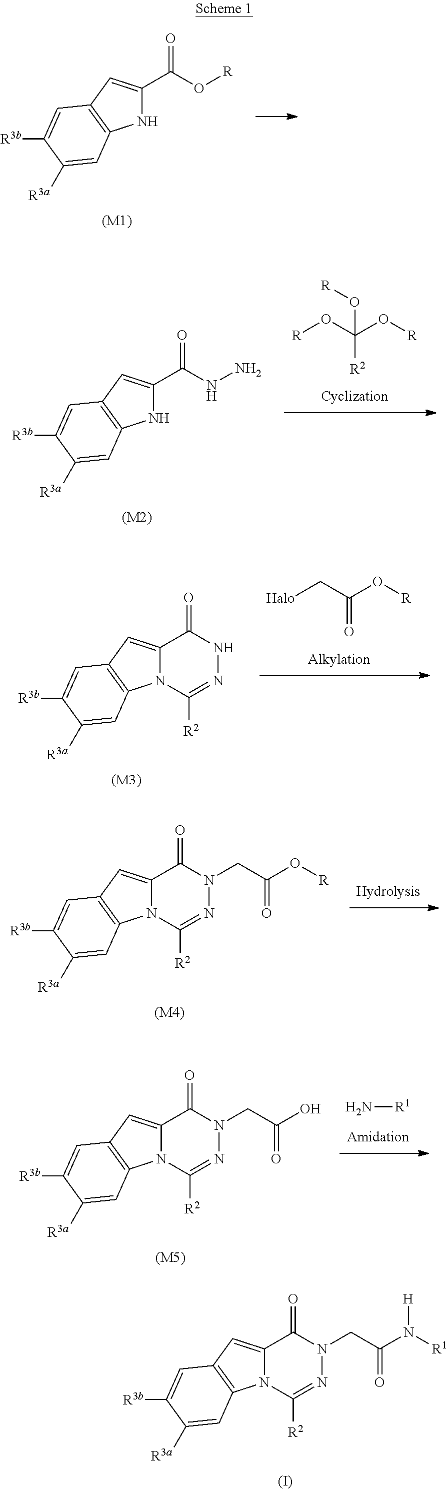

Compounds of the invention, as described herein, can be prepared by a reaction sequence shown in Scheme 1 (above), whereby an appropriately substituted indole (M1), wherein R is C₁₋₄ alkyl (and R^{3a} and R^{3b} are as defined herein), is reacted with hydrazine to give hydrazide (M2), which is then cyclized by reaction with an appropriate orthoester, wherein R is C₁₋₄alkyl (and R² is as defined herein), in the presence of a Lewis acid, e.g. aluminum isopropoxide, to the triazinone (M3) (also referred to herein as compound of formula (IV)) which is then alkylated with an appropriate alkyl haloacetate, wherein R is C₁₋₄ alkyl, in the presence of abase, e.g. K₂CO₃, a nucleophilic catalyst, e.g. KI and a crown ether, e.g. 18-crown-6, to provide ester (M4) which is typically cleaved e. g. under basic conditions, e.g. aqueous LiOH in THF or NaOH in MeOH to yield the acid intermediate (M5) (also referred to herein as compound of formula (II)), followed by amidation with R¹—NH₂ (wherein if R¹ has a functional group such as OH, NH₂, CO₂H, such group is optionally protected) using standard coupling conditions, e.g. 1-propanephosphonic anhydride and a base, e.g. triethylamine, optionally followed by an additional deprotection step to provide a compound of Formula (I), or a pharmaceutically acceptable salt thereof.

Further the following transformations, depicted in Schemes 2, 3 and 4 below, show versatility in allowing introduction of other substituents at the R² position of such intermediates too (as well as for final compounds):

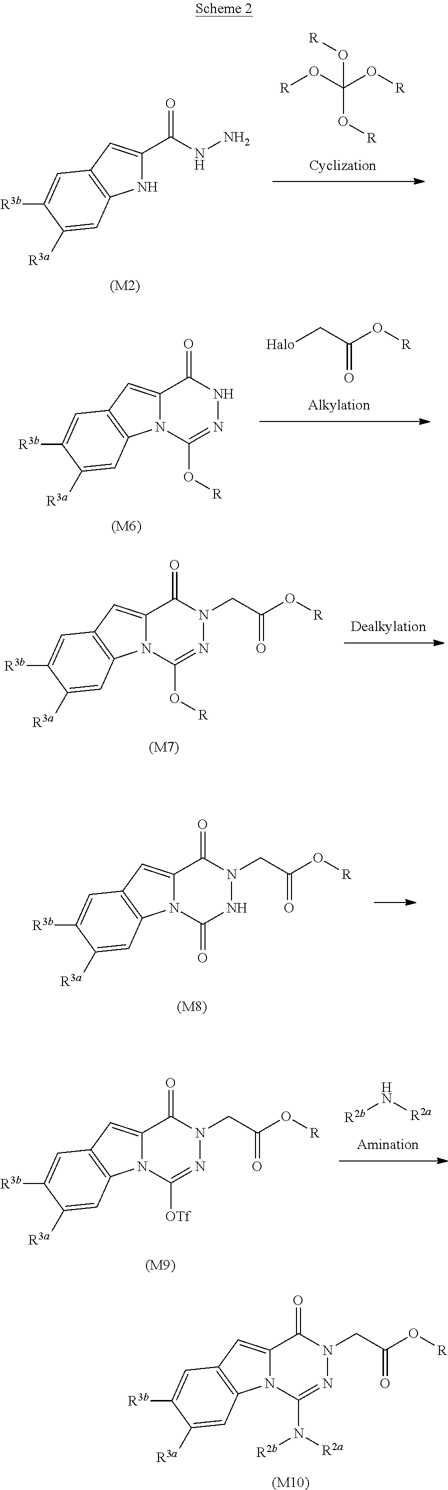

In Scheme 2 (above), whereby an appropriately substituted hydrazide (M2), wherein R^{3a} and R^{3b} are as defined herein, is cyclized by reaction with an appropriate orthoester, wherein R is C₁₋₄ alkyl, e.g. tetramethyl orthocarbonate, in the presence of a Lewis acid, e.g. aluminum isopropoxide, to the triazinone (M6) which is then alkylated with an appropriate alkyl haloacetate, wherein R is C₁₋₄ alkyl, in the presence of a base, e.g. K₂CO₃, a nucleophilic catalyst, e.g. KI and a crown ether, e.g. 18-crown-6, to provide ester (M7) which is then subjected to a ether-dealkylation reaction in the presence of a silyl halide, e.g. chlorotrimethylsilane and a nucleophilic catalyst, e.g. NaI, to yield intermediate (M8) which is then converted to a triflate intermediate (M9) in the presence of an electrophile, e.g. trifluoromethanesulfonic anhydride and a base, e.g. triethylamine, followed by an amination step with an appropriately substituted amine to give ester (M10), wherein R^{2a} and R^{2b} are defined herein, for e.g. are each, independently, C₁₋₄ alkyl.

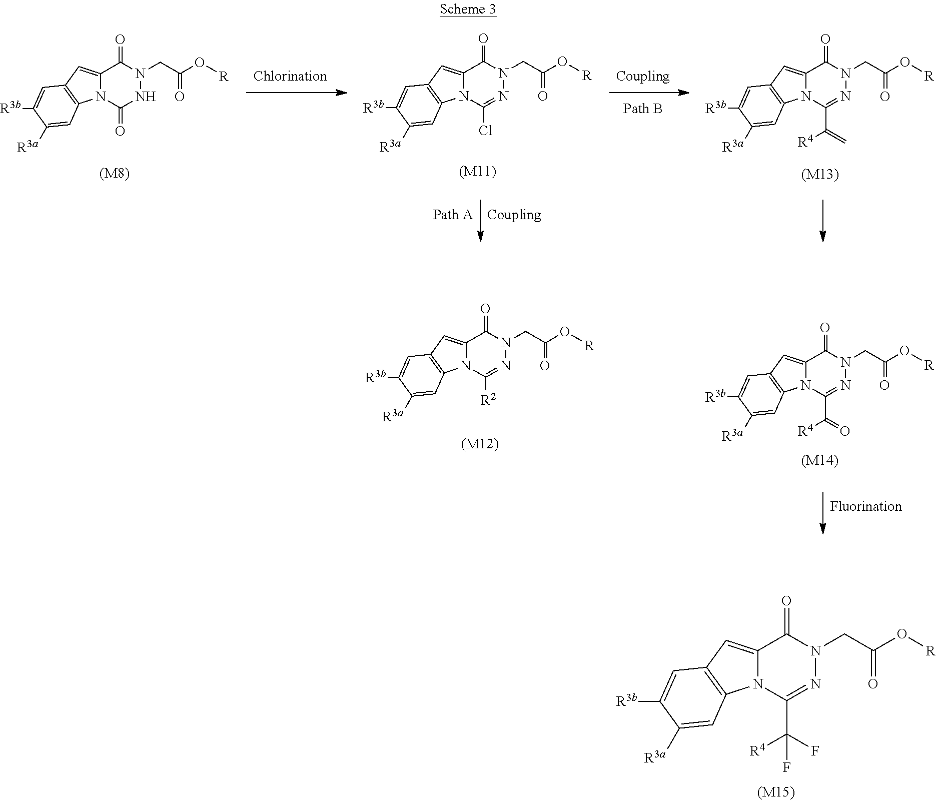

Alternatively, as per Scheme 3 above, intermediate (1′48), wherein R^{3a} and R^{3b} are as defined herein, is halogenated, e.g. with phosphorus (V) oxychloride, to give chloro-triazinone (M11), which is a very versatile intermediate as the chloro moiety may be replaced in a variety of coupling reactions. For instance, as described above, intermediate (M12) (Path A), wherein R² (in this scheme) is an hydrogen, an alkyl, a cycloalkyl or aryl/heteroaryl group, can be prepared by a Suzuki, Negishi or Stifle cross-coupling reactions, in the presence of an appropriate reagent such as a boronic acid, boronic ester, zincate or organotin compound, a suitable catalyst system, e.g. bis[tris(tert-butyl)phosphine]palladium or XPhos Pd G3, together with a suitable base, e.g. triethylamine. Additionally, intermediate (M12), wherein R² is a cyano group, may be prepared by a reaction with an appropriate salt, e.g. sodium cyanide and a base, e.g. 1,4-diazabicyclo[2.2.2]octane.

Alternatively, as described above in Scheme 3, an alkenyl intermediate (M13) (Path B), wherein R⁴ is hydrogen or an ethoxy group, may be prepared by a Stille cross-coupling reaction, in the presence of an appropriate organotin compound, e.g. bis[tris 1-ethoxy-1-(tributylstannyl)ethylene or tributyl(vinyl)tin, and a suitable catalyst system, e.g. bis[tris(tert-butyl)phosphine]palladium, followed by an oxidative cleavage using standard conditions, e.g. osmium tetroxide in combination with sodium metaperiodate and N-methylmorpholine N-oxide or hydrochloric acid for the enol ether cleavage to provide intermediate (M14), which is then reacted with a fluorinating reagent, e.g. bis(2-methoxyethyl) aminosulfur trifluoride or (diethylamino)sulfur trifluoride to yield intermediate (M15).

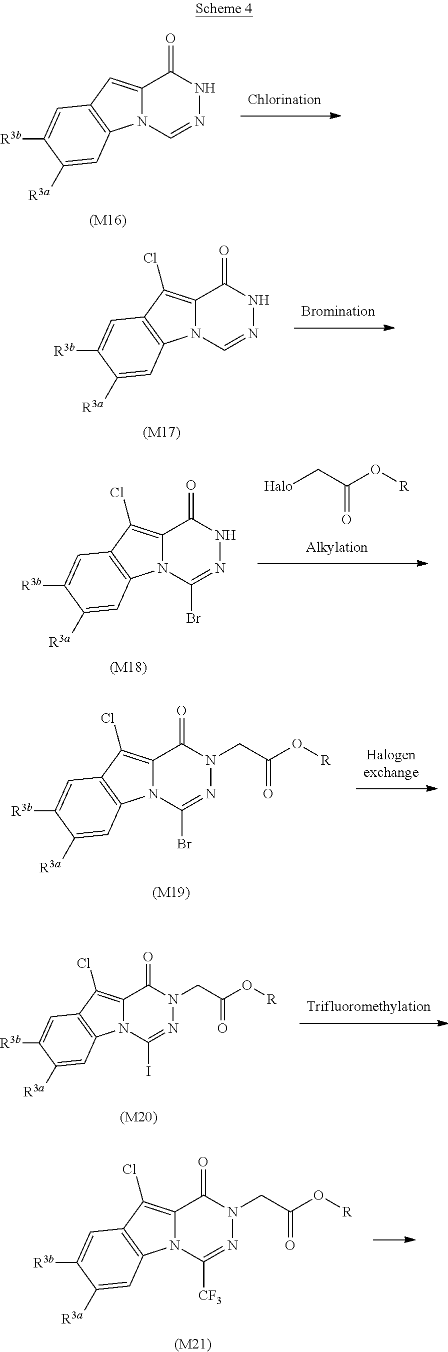

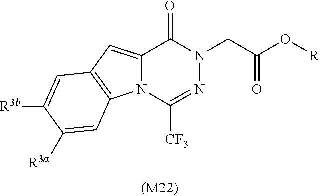

For instance, as per Scheme 4 above, the triazinone (M17) wherein R^{3a} and R^{3b} are as defined herein, may be prepared by a chlorination reaction, e.g. with N-chlorosuccinimide, of an appropriate substituted compound (M16), which is then followed by a bromination step, with e.g. benzyltrimethylammonium tribromide and a base, e.g. K₂CO₃ to provide dihalogenated intermediate (M18), which is then alkylated with an appropriate alkyl haloacetate, wherein R is C₁₋₄ alkyl, in the presence of a base, e.g. K₂CO₃, a nucleophilic catalyst, e.g. KI and a crown ether, e.g. 18-crown-6, to provide ester (M19), which is converted to the iodo-intermediate (M20) by an halogen exchange reaction in the presence of a iodine source, e.g. NaI, and a catalyst system, e.g. CuI and trans-N,N′-dimethylcyclohexane-1,2-diamine, which is then trifluoromethylated in the presence of a trifluoromethylating reagent, e.g. methyl 2,2-difluoro-2-(fluorosulfonyl) acetate and a catalyst, e.g. CuI to afford intermediate (M21), which is finally dechlorinated by hydrogenation in the presence of a catalyst, e.g. Pd/C and a base, e.g. triethylamine to yield intermediate (M22).

Certain intermediate compounds may be commercially available, may be known in the literature, or may be obtained either by analogy with the processes described herein, or by conventional synthetic procedures, in accordance with standard techniques, from available starting materials using appropriate reagents and reaction conditions.

Certain substituents on/in final compounds of the invention or relevant intermediates may be modified one or more times, after or during the processes described above by-way of methods that are well known to those skilled in the art. Examples of such methods include substitutions, reductions, oxidations, alkylations, acylations, hydrolyses, esterifications, etherifications, halogenations, nitrations or couplings.

Compounds of the invention may be isolated from their reaction mixtures using conventional techniques (e.g. recrystallisations, where possible under standard conditions).

It will be appreciated by those skilled in the art that, in the processes described above and hereinafter, the functional groups of intermediate compounds may need to be protected by protecting groups.

The need for such protection will vary depending on the nature of the remote functionality and the conditions of the preparation methods (and the need can be readily determined by one skilled in the art). Suitable amino-protecting groups include acetyl, trifluoroacetyl, t-butoxycarbonyl (BOC), benzyloxycarbonyl (CBz), 9-fluorenyl-methyleneoxycarbonyl (Fmoc) and 2,4,4-trimethylpentan-2-yl (which may be deprotected by reaction in the presence of an acid, e.g. HCl in water/alcohol (e.g. MeOH)) or the like. The need for such protection is readily determined by one skilled in the art. For example the a —C(O)O-tert-butyl ester moiety may serve as a protecting group for a —C(O)OH moiety, and hence the former may be converted to the latter for instance by reaction in the presence of a mild acid (e.g. TFA, or the like).

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

The compounds of the invention as prepared in the hereinabove described processes may be synthesized in the form of racemic mixtures of enantiomers which can be separated from one another following art-known resolution procedures. Those compounds of the invention that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of the invention involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

### Pharmacology

There is evidence for a role of NLRP3-induced IL-1 and IL-18 in the inflammatory responses occurring in connection with, or as a result of, a multitude of different disorders (Menu et al., Clinical and Experimental Immunology, 2011, 166, 1-15; Strowig et al., Nature, 2012, 481, 278-286). NLRP3 mutations have been found to be responsible for a set of rare autoinflammatory diseases known as CAPS (Ozaki et al., J. Inflammation Research, 2015, 8, 15-27; Schroder et al., Cell, 2010, 140: 821-832; Menu et al., Clinical and Experimental Immunology, 2011, 166, 1-15). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1 beta. NLRP3 has also been implicated in a number of autoinflammatory diseases, including pyogenic arthritis, pyoderma gangrenosum and acne (PAPA), Sweet's syndrome, chronic nonbacterial osteomyelitis (CNO), and acne vulgaris (Cook et al., Eur. J. Immunol., 2010, 40, 595-653).

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Braddock et al., Nat. Rev. Drug Disc. 2004, 3, 1-10; Inoue et al., Immunology, 2013, 139, 11-18; Coll et al., Nat. Med. 2015, 21(3), 248-55; Scott et al., Clin. Exp. Rheumatol. 2016, 34(1), 88-93), systemic lupus erythematosus and its complications such as lupus nephritis (Lu et al., J. Immunol., 2017, 198(3), 1119-29), and systemic sclerosis (Artlett et al., Arthritis Rheum. 2011, 63(11), 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 2014, 184: 42-54; Kim et al., Am. J. Respir. Crit. Care Med 2017, 196(3), 283-97). NLRP3 has also been suggested to have a role in a number of central nervous system conditions, including Multiple Sclerosis (MS), Parkinson's disease (PD), Alzheimer's disease (AD), dementia, Huntington's disease, cerebral malaria, brain injury from pneumococcal meningitis (Walsh et al., Nature Reviews, 2014, 15, 84-97; and Dempsey et al., Brain. Behav. Immun. 2017, 61, 306-16), intracranial aneurysms (Zhang et al., J. Stroke and Cerebrovascular Dis., 2015, 24, 5, 972-9), and traumatic brain injury (Ismael et al., J. Neurotrauma., 2018, 35(11), 1294-1303). NLRP3 activity has also been shown to be involved in various metabolic diseases including type 2 diabetes (T2D) and its organ-specific complications, atherosclerosis, obesity, gout, pseudo-gout, metabolic syndrome (Wen et al., Nature Immunology, 2012, 13, 352-357; Duewell et al., Nature, 2010, 464, 1357-1361; Strowig et al., Nature, 2014, 481, 278-286), and non-alcoholic steatohepatitis (Mridha et al., J. Hepatol. 2017, 66(5), 1037-46). A role for NLRP3 via EL-1 beta has also been suggested in atherosclerosis, myocardial infarction (van Hout et al., Eur. Heart J. 2017, 38(11), 828-36), heart failure (Sano et al., J. Am. Coll. Cardiol. 2018, 71(8), 875-66), aortic aneurysm and dissection (Wu et al., Arteriosc/er. Thromb. Vase. Biol., 2017, 37(4), 694-706), and other cardiovascular events (Ridker et al., N. Engl. J. Med., 2017, 377(12), 1119-31).

Other diseases in which NLRP3 has been shown to be involved include: ocular diseases such as both wet and dry age-related macular degeneration (Doyle et al., Nature Medicine, 2012, 18, 791-798; Tarallo et al., Cell 2012, 149(4),847-59), diabetic retinopathy (Loukovaara et al., Acta Ophthalmol., 2017, 95(8), 803-8), non-infectious uveitis and optic nerve damage (Puyang et al., Sci. Rep. 2016, 6, 20998); liver diseases including non-alcoholic steatohepatitis (NASH) and acute alcoholic hepatitis (Henao-Meija et al., Nature, 2012, 482, 179-185); inflammatory reactions in the lung and skin (Primiano et al., J. Immunol. 2016, 197(6),2421-33) including contact hypersensitivity (such as bullous pemphigoid (Fang et al., J Dermatol Sci. 2016, 83(2), 116-23)), atopic dermatitis (Niebuhr et al., Allergy, 2014, 69(8), 1058-67), Hidradenitis suppurativa (Alikhan et al., J. Am. Acad. Dermatol., 2009, 60(4), 539-61), and sarcoidosis (Jager et al., Am. J. Respir. Crit. Care Med., 2015, 191, A5816); inflammatory reactions in the joints (Braddock et al., Nat. Rev. Drug Disc, 2004, 3, 1-10); amyotrophic lateral sclerosis (Gugliandolo et al., Int. J. Mol. Sci., 2018, 19(7), E1992); cystic fibrosis (lannitti et al., Nat. Commun., 2016, 7, 10791); stroke (Walsh et al., Nature Reviews, 2014, 15, 84-97); chronic kidney disease (Granata et al., PLoS One 2015, 10(3), eoi22272); and inflammatory bowel diseases including ulcerative colitis and Crohn's disease (Braddock et al., Nat. Rev. Drug Disc, 2004, 3, 1-10; Neudecker et al., J. Exp. Med. 2017, 214(6), 1737-52; Lazaridis et al., Dig. Dis. Sci. 2017, 62(9),2348-56). The NLRP3 inflammasome has been found to be activated in response to oxidative stress. NLRP3 has also been shown to be involved in inflammatory hyperalgesia (Dolunay et al., Inflammation, 2017, 40, 366-86).

Activation of the NLRP3 inflammasome has been shown to potentiate some pathogenic infections such as influenza and Leishmaniasis (Tate et al., Sci Rep., 2016, 10(6), 27912-20; Novias et al., PLOS Pathogens 2017, 13(2), e1006196).

NLRP3 has also been implicated in the pathogenesis of many cancers (Menu et al., Clinical and Experimental Immunology, 2011, 166, 1-15). For example, several previous studies have suggested a role for IL-1 beta in cancer invasiveness, growth and metastasis, and inhibition of IL-1 beta with canakinumab has been shown to reduce the incidence of lung cancer and total cancer mortality in a randomised, double-blind, placebo-controlled trial (Ridker et al., Lancet., 2017, 390(10105), 1833-42). Inhibition of the NLRP3 inflammasome or IL-1 beta has also been shown to inhibit the proliferation and migration of lung cancer cells in vitro (Wang et al., Onco Rep., 2016, 35(4), 2053-64). A role for the NLRP3 inflammasome has been suggested in myelodysplastic syndromes, myelofibrosis and other myeloproliferative neoplasms, and acute myeloid leukemia (AML) (Basiorka et al., Blood, 2016, 128(25), 2960-75.) and also in the carcinogenesis of various other cancers including glioma (Li et al., Am. J. Cancer Res. 2015, 5(1), 442-9), inflammation-induced tumors (Allen et al., J. Exp. Med. 2010, 207(5), 1045-56; Hu et al., PNAS., 2010, 107(50), 21635-40), multiple myeloma (Li et al., Hematology, 2016 21(3), 144-51), and squamous cell carcinoma of the head and neck (Huang et al., J. Exp. Clin. Cancer Res., 2017, 36(1), 116). Activation of the NLRP3 inflammasome has also been shown to mediate chemoresistance of tumor cells to 5-Fluorouracil (Feng et al., J. Exp. Clin. Cancer Res., 2017, 36(1), 81), and activation of NLRP3 inflammasome in peripheral nerve contributes to chemotherapy-induced neuropathic pain (Jia et al., Mol. Pain., 2017, 13, 1-11). NLRP3 has also been shown to be required for the efficient control of viruses, bacteria, and fungi.

The activation of NLRP3 leads to cell pyroptosis and this feature plays an important part in the manifestation of clinical disease (Yan-gang et al, Cell Death and Disease, 2017, 8(2), 2579; Alexander et al., Hepatology, 2014, 59(3), 898-910; Baldwin et al, J. Med. Chem., 2016, 59(5), 1691-1710; Ozaki et al., J. Inflammation Research, 2015, 8, 15-27; Zhen et al., Neuroimmunology Neuroinflammation, 2014, 1(2),60-65; Mattia et al, J. Med Chem., 2014, 57(24), 10366-82; Satoh et al., Cell Death and Disease, 2013, 4, 644). Therefore, it is anticipated that inhibitors of NLRP3 will block pyroptosis, as well as the release of pro-inflammatory cytokines (e.g. IL-1 beta) from the cell.

Hence, the compounds of the invention, as described herein (and, where applicable, without the provisos) (e.g. in any of the embodiments described herein, including by the examples, and/or in any of the forms described herein, e.g. in a salt form or free form, etc) exhibit valuable pharmacological properties, e.g. NLRP3 inhibiting properties on the NLRP3 inflammasome pathway e.g. as indicated in vitro tests as provided herein, and are therefore indicated for therapy or for use as research chemicals, e.g. as tool compounds. Compounds of the invention (and, where applicable, without the provisos) may be useful in the treatment of an indication selected from: inflammasome-related diseases/disorders, immune diseases, inflammatory diseases, auto-immune diseases, or auto-inflammatory diseases, for example, of diseases, disorders or conditions in which NLRP3 signaling contributes to the pathology, and/or symptoms, and/or progression, and which may be responsive to NLRP3 inhibition and which may be treated or prevented, according to any of the methods/uses described herein, e.g. by use or administration of a compound of the invention, and, hence, in an embodiment, such indications may include:
I. Inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity. Examples of inflammation that may be treated or prevented include inflammatory responses occurring in connection with, or as a result of:
   a. a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
   b. a joint condition such as osteoarthritis, systemic juvenile idiopathic arthritis, adult-onset Still's disease, relapsing polychondritis, rheumatoid arthritis, juvenile chronic arthritis, crystal induced arthropathy (e.g. pseudo-gout, gout), or a seronegative spondyloarthropathy (e.g. ankylosing spondylitis, psoriatic arthritis or Reiter's disease);
   c. a muscular condition such as polymyositis or myasthenia gravis;
   d. a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
   e. a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
   f. a vascular condition such as atherosclerosis, Behcet's disease, vasculitides, or Wegener's granulomatosis;
   g. an immune condition, e.g. autoimmune condition, such as systemic lupus erythematosus (SLE), Sjogren's syndrome, systemic sclerosis, Hashimoto's thyroiditis, type I diabetes, idiopathic thrombocytopenia purpura, or Graves disease;
   h. an ocular condition such as uveitis, allergic conjunctivitis, or vernal conjunctivitis;
   i. a nervous condition such as multiple sclerosis or encephalomyelitis;
   j. an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;
   k. a renal condition such as mesangial proliferative glomerulonephritis, nephrotic syndrome, nephritis, glomerular nephritis, acute renal failure, uremia, or nephritic syndrome;
   l. a lymphatic condition such as Castleman's disease;
   m. a condition of, or involving, the immune system, such as hyper lgE syndrome, lepromatous leprosy, familial hemophagocytic lymphohistiocytosis, or graft versus host disease;
   n. a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepaiitis (ASH) or primary biliary cirrhosis;
   o. a cancer, including those cancers listed herein below;
   p. a burn, wound, trauma, haemorrhage or stroke;
   q. radiation exposure;
   r. obesity; and/or
   s. pain such as inflammatory hyperalgesia;
II. Inflammatory disease, including inflammation occurring as a result of an inflammatory disorder, e.g. an autoinflammatory disease, such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD);
III. Immune diseases, e.g. auto-immune diseases, such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Beliefs disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, giant cell arteritis, vitiligo or vulvodynia; IV. Cancer including lung cancer, renal cell carcinoma, non-small cell lung carcinoma (NSCLC), Langerhans cell histiocytosis (LCH), myeloproliferative neoplams (MPN), pancreatic cancer, gastric cancer, myelodysplastic syndrome (MOS), leukaemia including acute lymphocytic leukaemia (ALL) and acute myeloid leukaemia (AML), promyelocytic leukemia (APML, or APL), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;
V. Infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), papillomavirus, or SARS-CoV-2) bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), and prion infections;
VI. Central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, multiple sclerosis, and amyotrophic lateral sclerosis;
VII. Metabolic diseases such as type 2 diabetes (T2D), atherosclerosis, obesity, gout, and pseudo-gout;
VIII. Cardiovascular diseases such as hypertension, ischaemia, reperfusion injury including post-Ml ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, cardiovascular risk reduction (CvRR), and pericarditis including Dressler's syndrome;
IX. Respiratory diseases including chronic obstructive pulmonary disorder (COPD), asthma such as allergic asthma and steroid-resistant asthma, asbestosis, silicosis, nanoparticle induced inflammation, cystic fibrosis, and idiopathic pulmonary fibrosis;
X. Liver diseases including non-alcoholic fatty liver disease (NAFLD) and nonalcoholic steatohepatitis (NASH) including advanced fibrosis stages F3 and F4, alcoholic fatty liver disease (AFLD), and alcoholic steatohepatitis (ASH);
XI. Renal diseases including acute kidney disease, hyperoxaluria, chronic kidney disease, oxalate nephropathy, nephrocalcinosis, glomerulonephritis, and diabetic nephropathy;
XII. Ocular diseases including those of the ocular epithelium, age-related macular degeneration (AMO) (dry and wet), uveitis, corneal infection, diabetic retinopathy, optic nerve damage, dry eye, and glaucoma;
XIII. Skin diseases including dermatitis such as contact dermatitis and atopic dermatitis, contact hypersensitivity, sunburn, skin lesions, hidradenitis suppurativa (HS), other cyst-causing skin diseases, and acne conglobata;
XIV. Lymphatic conditions such as lymphangitis, and Castleman's disease;
XV. Psychological disorders such as depression, and psychological stress;
XVI. Graft versus host disease;
XVII. Bone diseases including osteoporosis, osteopetrosis;
XVIII. Blood disease including sickle cell disease;
XIX. Allodynia including mechanical allodynia; and
XX. Any disease where an individual has been determined to carry a germline or somatic non-silent mutation in NLRP3.

More specifically the compounds of the invention (without the provisos) may be useful in the treatment of an indication selected from: inflammasome-related diseases/disorders, immune diseases, inflammatory diseases, auto-immune diseases, or auto-inflammatory diseases, for example, autoinflammatory fever syndromes (e.g., cryopyrin-associated periodic syndrome), sickle cell disease, systemic lupus erythematosus (SLE), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimers disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemias, myelodysplastic syndromes (MOS), myelofibrosis). In particular, autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis).

In particular, compounds of the invention (without the provisos), may be useful in the treatment of a disease or disorder selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). Thus, as a further aspect, the present invention provides the use of a compound of the invention (without the provisos) (hence, including a compound as defined by any of the embodiments/forms/examples herein) in therapy. In a further embodiment, the therapy is selected from a disease, which may be treated by inhibition of NLRP3 inflammasome. In another embodiment, the disease is as defined in any of the lists herein. Hence, there is provided any one of the compounds of the invention (without the provisos) described herein (including any of the embodiments/forms/examples) for use in the treatment of any of the diseases or disorders described herein (e.g. as described in the aforementioned lists).

### Pharmaceutical Compositions and Combinations

In an embodiment, the invention also relates to a composition comprising a pharmaceutically acceptable carrier and, as active ingredient, a therapeutically effective amount of a compound of the invention (without the provisos). The compounds of the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

In an embodiment, and depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

In an embodiment, there is provided a combination comprising a therapeutically effective amount of a compound of the invention (without the provisos), according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH4) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention (without the provisos), e.g. for use in the treatment of a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in respect of compounds of the invention (without the provisos), but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein), for instance, in an embodiment, the combination may be a kit of parts. Such combinations may be referred to as “pharmaceutical combinations”. The route of administration for a compound of the invention (without the provisos) as a component of a combination may be the same or different to the one or more other therapeutic agent(s) with which it is combined. The other therapeutic agent is, for example, a chemical compound, peptide, antibody, antibody fragment or nucleic acid, which is therapeutically active or enhances the therapeutic activity when administered to a patient in combination with a compound of the invention (without the provisos).

The weight ratio of (a) the compound according to the invention and (b) the other therapeutic agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The pharmaceutical composition or combination of the present invention can be in unit dosage of about 1-1000 mg of active ingredient(s) for a subject of about 50-70 kg, or about 1-500 mg, or about 1-250 mg, or about 1-150 mg, or about 1-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10−³ molar and 10−⁹ molar concentrations. A therapeutically effective amount in vivo may range depending on the route of administration, between about 0.1-500 mg/kg, or between about 1-100 mg/kg.

As used herein, term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The term “subject” as used herein, refers to an animal, preferably a mammal, most preferably a human, for example who is or has been the object of treatment, observation or experiment.

The term “therapeutically effective amount” as used herein, means that amount of compound of the invention (including, where applicable, form, composition, combination comprising such compound of the invention) elicits the biological or medicinal response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by NLRP3, or (ii) associated with NLRP3 activity, or (iii) characterised by activity (normal or abnormal) of NLRP3; or (2) reduce or inhibit the activity of NLRP3; or (3) reduce or inhibit the expression of NLRP3. In another non-limiting embodiment, the term “a therapeutically effective amount” refers to the amount of the compound of the present invention that, when administered to a cell, or a tissue, or a non-cellular-biological material, or a medium, is effective to at least partially reduce or inhibit the activity of NLRP3; or at least partially reduce or inhibit the expression of NLRP3.

As used herein, the term “inhibit”, “inhibition” or “inhibiting” refers to the reduction or suppression of a given condition, symptom, or disorder, or disease, or a significant decrease in the baseline activity of a biological activity or process. Specifically, inhibiting NLRP3 or inhibiting NLRP3 inflammasome pathway comprises reducing the ability of NLRP3 or NLRP3 inflammasome pathway to induce the production of IL-1 and/or IL-18. This can be achieved by mechanisms including, but not limited to, inactivating, destabilizing, and/or altering distribution of NLRP3.

As used herein, the term “NLRP3” is meant to include, without limitation, nucleic acids, polynucleotides, oligonucleotides, sense and anti-sense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals. The single components may be packaged in a kit or separately. One or both of the components (e.g. powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “pharmaceutical combination” as used herein refers to either a fixed combination in one dosage unit form, or non-fixed combination or a kit of parts for the combined administration where two or more therapeutic agents may be administered independently at the same time or separately within time intervals. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g. tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Summary of Pharmacology. Uses. Compositions and Combinations

In an embodiment, there is provided a pharmaceutical composition comprising a therapeutically effective amount of a compound of the invention (without the provisos), according to any one of the embodiments described herein, and a pharmaceutically acceptable carrier (including one or more pharmaceutically acceptale carriers).

In an embodiment, there is provided a compound of the invention (without the provisos), according to any one of the embodiments described herein, for use as a medicament.

In an embodiment, there is provided a compound of the invention (without the provisos), according to any one of the embodiments described herein (and/or pharmaceutical compositions comprising such compound of the invention (without the provisos), according to any one of the embodiment described herein) for use: in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder, in inhibiting NLRP3 inflammasome activity (including in a subject in need thereof); and/or as an NLRP3 inhibitor.

In an embodiment, there is provided a use of compounds of the invention (without the provisos), according to any one of the embodiments described herein (and/or pharmaceutical compositions comprising such compound of the invention (without the provisos), according to any one of the embodiment described herein): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder, in inhibiting NLRP3 inflammasome activity (including in a subject in need thereof); and/or as an NLRP3 inhibitor.

In an embodiment, there is provided use of compounds of the invention (without the provisos), according to any one of the embodiments described herein (and/or pharmaceutical compositions comprising such compound of the invention (without the provisos), according to any one of the embodiment described herein), in the manufacture of a medicament for the treatment of a disease or disorder associated with NLRP3 activity (including inflammasome activity); the treatment of a disease or disorder in which the NLRP3 signalling contributes to the pathology, and/or symptoms, and/or progression, of said disease/disorder; and/or inhibiting NLRP3 inflammasome activity (including in a subject in need thereof).

In an embodiment, there is provided a method of treating a disease or disorder in which the NLRP3 signalling contributes to the pathology, and/or symptoms, and/or progression, of said disease/disorder, comprising administering a therapeutically effective amount of a compound of the invention (without the provisos), according to any one of the embodiments described herein (and/or pharmaceutical compositions comprising such compound of the invention (without the provisos), according to any one of the embodiment described herein), for instance to a subject (in need thereof). In a further embodiment, there is provided a method of inhibiting the NLRP3 inflammasome activity in a subject (in need thereof), the method comprising administering to the subject in need thereof a therapeutically effective amount of a compound of the invention, according to any one of the embodiments described herein (and/or pharmaceutical compositions comprising such compound of the invention, according to any one of the embodiment described herein).

In all relevant embodiment of the invention, where a disease or disorder is mentioned (e.g. hereinabove), for instance a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, then such disease may include inflammasome-related diseases or disorders, immune diseases, inflammatory diseases, auto-immune diseases, or auto-inflammatory diseases. In a further embodiment, such disease or disorder may include autoinflammatory fever syndromes (e.g cryopyrin-associated periodic syndrome), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g acute, chronic), kidney related diseases (e.g. hyperoxaluria, lupus nephritis, Type II/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukaemia, myelodysplastic syndromes (MOS), myelofibrosis). In a particular aspect, such disease or disorder is selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). In a particular embodiment, the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

In an embodiment, there is provided a combination comprising a therapeutically effective amount of a compound of the invention (without the provisos), according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH4) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention (without the provisos), e.g. for use in the treatment of a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in respect of compounds of the invention (without the provisos), but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

For instance, compounds of the invention may have the advantage that they have a good or an improved thermodynamic solubility (e.g. compared to compounds known in the prior art; and for instance as determined by a known method and/or a method described herein). Compounds of the invention may have the advantage that they will block pyroptosis, as well as the release of pro-inflammatory cytokines (e.g. IL-1β) from the cell. Compounds of the invention may also have the advantage that they avoid side-effects, for instance as compared to compounds of the prior art, which may be due to selectivity of NLRP3 inhibition. Compounds of the invention may also have the advantage that they have good or improved in vivo pharmacokinetics and oral bioavailabilty. They may also have the advantage that they have good or improved in vivo efficacy. Specifically, compounds of the invention may also have advantages over prior art compounds when compared in the tests outlined hereinafter (e.g. in Examples C and D).

### General Preparation and Analytical Processes

The compounds according to the invention can generally be prepared by a succession of steps, each of which may be known to the skilled person or described herein.

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SFC).

The starting materials and the intermediates are compounds that are either commercially available or may be prepared according to conventional reaction procedures generally known in the art.

### Analytical Part

### LC-MS (LIQUID CHRoMAToGRAPHY/MAS SPECROMETRY)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

TABLE
LCMS Method codes (Flow expressed in mL/min; column temperature (T)
in ° C.; Run time in minutes).

| | | | | | Flow | |
|---|---|---|---|---|---|---|
| Method | | | | | Col | Run |
| code | Instrument | column | mobile phase | gradient | T | time |
| Method A | Waters: | Agilent: | A: 95% | From 95% | 0.8 | 2.5 |
| | Acquity ® | RRHD | CH₃COONH₄ | A to 5% A | 50 | |
| | IClass | (1.8 μm, | 6.5 mM + 5% | in 2.0 min, | | |
| | UPLC ®-DAD | 2.1 × 50 mm) | CH₃CN, B: | held for | | |
| | and SQD | | CH₃CN | 0.5 min | | |
| Method B | Waters: | Waters: | A: 95% | From 95% | 0.8 | 2.5 |
| | Acquity ® | BEH C18 | CH₃COONH₄ | A to 5% A | 50 | |
| | UPLC ®-DAD | (1.7 μm, | 6.5 mM + 5% | in 2.0 min, | | |
| | and SQD | 2.1 × 50 mm) | CH₃CN, B: | held for | | |
| | | | CH₃CN | 0.5 min | | |
| Method C | Waters: | Waters: | A: 95% | From 95% | 1 | 2 |
| | Acquity ® | BEH C18 | CH₃COONH₄ | A to 40% | 50 | |
| | IClass | (1.7 μm, | 6.5 mM + 5% | A in 1.2 min, | | |
| | UPLC ®-DAD | 2.1 × 50 mm) | CH₃CN, B: | to 5% A in | | |
| | and Xevo | | CH₃CN | 0.6 min, held | | |
| | G2-S QTOF | | | for 0.2 min | | |
| Method D | Waters: | Waters: | A: 95% | From 95% | 1 | 5 |
| | Acquity ® | BEH C18 | CH₃COONH₄ | A to 5% A | 50 | |
| | IClass | (1.7 μm, | 6.5 mM + 5% | in 4.6 min, | | |
| | UPLC ®-DAD | 2.1 × 50 | CH₃CN, B: | held for | | |
| | and Xevo G2-S | mm) | CH₃CN | 0.4 min | | |
| | QTOF | | | | | |
| Method E | Waters: | Waters: | A: 95% | From 95% | 1 | 2 |
| | Acquity ® | BEH C18 | CH₃COONH₄ | A to 40% A | 50 | |
| | IClass | (1.7 μm, | 6.5 mM + 5% | in 1.2 min, to | | |
| | UPLC ®-DAD | 2.1 × 50 | CH₃CN, B: | 5% A in 0.6 | | |
| | and Xevo G2-S | mm) | CH₃CN | min, held for | | |
| | QTOF | | | 0.2 min | | |
| Method F | Waters: | Waters: | A: 10 mM | From | 0.6 | 3.5 |
| | Acquity ® | BEH | CH₃COONH₄ | 100% A to | 55 | |
| | UPLC ®-DAD | (1.8 μm, | in 95% | 5% A in | | |
| | and SQD | 2.1*100 | H₂O + 5% | 2.10 min, | | |
| | | mm) | CH₃CN, | to 0% A | | |
| | | | B: CH₃CN | in 0.9 min, | | |
| | | | | to 5% A | | |
| | | | | in 0.5 min | | |
| Method G | Waters: | Waters: | A: 95% | From 95% A | 0.8 | 5 |
| | Acquity ® | BEH C18 | CH₃COONH₄ | to 5% A in | 50 | |
| | UPLC ®-DAD | (1.7 μm, | 6.5 mM + 5% | 4.5 min, | | |
| | and SQD | 2.1 × 50 | CH₃CN, B: | held for | | |
| | | mm) | CH₃CN | 0.5 min | | |
| Method H | Waters: | Agilent: | A: 95% | From | 0.8 | 5 |
| | Acquity ® | RRHD | CH₃COONH₄ | 95% A to | 50 | |
| | IClass | (1.8 μm, | 6.5 mM + 5% | 5% A in | | |
| | UPLC ®-DAD | 2.1 × 50 | CH₃CN, B: | 4.5 min, | | |
| | and SQD | mm) | CH₃CN | held for | | |
| | | | | 0.5 min | | |
| Method I | Waters: | Waters: | A: 95% | From | 1 | 5 |
| | Acquity ® | BEH C18 | CH₃COONH₄ | 95% A to | 50 | |
| | IClass | (1.7 μm, | 6.5 mM + 5% | 5% A in | | |
| | UPLC ®- | 2.1 × 50 | CH₃CN, B: | 4.6 min, | | |
| | DAD and | mm) | CH₃CN | held for | | |
| | XevoG2-s | | | 0.4 min | | |
| | QTOF | | | | | |
| Method J | Waters: | Waters: | A: 95% | From | 1 | 3 |
| | Acquity ® | XBridge | HCO₃NH₄ | 90% A to | 25 | |
| | HClass | C18 (2.5 μm, | 2.5 g/L (32 | 0% A in | | |
| | UPLC ®-DAD | 2.1 × 50 | mM), B: | 2.0 min, | | |
| | and QDa | mm) | CH₃CN | held for | | |
| | | | | 0.5 min, | | |
| | | | | to 90% A | | |
| | | | | in 0.2 | | |
| | | | | min, held | | |
| | | | | for 0.3 min | | |
| Method K | Waters: | Waters: | A: 95% | From | 0.8 | 3 |
| | Acquity ® | XBridge | HCO₃NH₄ | 90% A to | 25 | |
| | HClass | C18 (2.5 μm, | 2.5 g/L (32 | 0% A in 3.0 | | |
| | UPLC ®-DAD | 2.1 × 50 | mM), B: | min, held for | | |
| | and QDa | mm) | CH₃CN | 0.5 min, to | | |
| | | | | 90% A in 0.7 | | |
| | | | | min, held | | |
| | | | | for 0.8 min | | |
| Method L | Waters: | Waters: | A: 10 mM | From 100% A | 0.6 | 3.5 |
| | Acquity ® | BEH | HCO3NH4 | to 5% A in | 55 | |
| | UPLC ®-DAD | (1.8 μm, | in 95% | 2.10 min, to | | |
| | and SQD | 2.1*100 | H2O + 5% | 0% A in 0.9 | | |
| | | mm) | CH3CN, | min, to 5% A | | |
| | | | B: CH3CN | in 0.5 min | | |
| Method M | Agilent 1260 | YMC-pack | A: 0.1% | From 95% A | 2.6 | 6.8 |
| | Infinity | ODS-AQ | HCOOH in. | to 5% A in | 35 | |
| | DAD TOF- | C18 (50 × | H2O | 4.8 min, held | | |
| | LC/MS | 4.6 mm, 3 | B: CH3CN | for 1.0 min, | | |
| | G6224A | μm) | | to 95% A | | |
| | | | | in 0.2 min. | | |
| Method N | Agilent 1100 | YMC-pack | A: 0.1% | From 95% A | 2.6 | 6.2 |
| | HPLC DAD | ODS-AQ | HCOOH in | to 5% A in | 35 | |
| | LC/MS | C18 (50 × | H2O | 4.8 min, held | | |
| | G1956A | 4.6 mm, 3 | B: CH3CN | for 1.0 min, | | |
| | | μm) | | to 95% A | | |
| | | | | in 0.2 min | | |
| Method O | Agilent: | Waters: | A: | From 90% A | 0.8 | 5 |
| | 1290 Infinity | XBridgeC18 | HCO3NH4 | to 0% A in | 25 | |
| | II-DAD and | (2.5 μm, | 2.5 g/L (32 | 3.0 min, held | | |
| | MSD/XT | 2.1 × 50 | mM) | for 0.5 min, | | |
| | | mm) | B: CH3CN | to 90% A in | | |
| | | | | 0.7 min, held | | |
| | | | | for 0.8 min | | |

### NMR

For a number of compounds, ¹H NMR spectra were recorded on a Bruker Avance M spectrometer operating at 300 or 400 MHz, on a Bruker Avance III-HD operating at 400 MHz, on a Bruker Avance NEO spectrometer operating at 400 MHz, on a Bruker Avance Neo spectrometer operating at 500 MHz, or on a Bruker Avance 600 spectrometer operating at 600 MHz, using CHLOROFORM-d (deuterated chloroform, CDCl₃), DMSO-d₆ (deuterated DMSO, dimethyl-d6 sulfoxide), METHANOL-d₄ (deuterated methanol), BENZENE-d₆ (deuterated benzene, C₆D₆) or ACETONE-d₆(deuterated acetone, (CD₃)₂CO) as solvents. Chemical shifts (□) are reported in parts per million (ppm) relative to tetramethylsilane (TMS), which was used as internal standard.

### Melting Points

Values are either peak values or melt ranges, and are obtained with experimental uncertainties that are commonly associated with this analytical method. For a number of compounds, melting points were determined with a DSC823e (Mettler Toledo) apparatus. Melting points were measured with a temperature gradient of 10° C./minute. Standard maximum temperature was 300° C.

### Experimental Part

Hereinafter, the term “m.p.” means melting point, “aq.” means aqueous, “r.m.” means reaction mixture, “rt” means room temperature, ‘DIPEA’ means N,N-diiso-propylethylamine, “DIPE” means diisopropylether, ‘THF’ means tetrahydrofuran, ‘DMF’ means dimethylformamide, ‘DCM’ means dichloromethane, “EtOH” means ethanol ‘EtOAc’ means ethyl acetate, “AcOH” means acetic acid, “iPrOH” means isopropanol, “iPrNH2” means isopropylamine, “MeCN” or “ACN” means acetonitrile, “MeOH” means methanol, “Pd(OAc)₂” means palladium(II)diacetate, “rac” means racemic, ‘sat.’ means saturated, ‘SFC’ means supercritical fluid chromatography, ‘SFC-MS’ means supercritical fluid chromatography/mass spectrometry, “LC-MS” means liquid chromatography/mass spectrometry, “GCMS” means gas chromatography/mass spectrometry, “HPLC” means high-performance liquid chromatography, “RP” means reversed phase, “UPLC” means ultra-performance liquid chromatography, “Rₜ” (or “RT”) means retention time (in minutes), “[M+H]⁺” means the protonated mass of the free base of the compound, “DAST” means diethylaminosulfur trifluoride, “DMTMM” means 4-(4,6-dimethoxy-1,3,5-triazin-2-yl)-4-methylmorpholinium chloride, “HATU” means O-(7-azabenzotriazol-1-yl)-N,N,N′,N′-tetramethyluronium hexafluorophosphate (1-[bis(dimethylamino)methylene]-1H-1,2,3-triazolo[4,5-b]pyridinium 3-oxide hexafluorophosphate), “Xantphos” means (9,9-dimethyl-9H-xanthene-4,5-diyl)bis[diphenylphosphine], “TBAT” means tetrabutyl ammonium triphenyldifluorosilicate, “TFA” means trifuoroacetic acid, “Et₂O” means diethylether, “DMSO” means dimethylsulfoxide, “SiO₂” means silica, “XPhos Pd G3” means (2-dicyclohexylphosphino-2′,4′,6′-triisopropyl-1,1′-biphenyl)[2-(2′-amino-1,1′-biphenyl)]palladium(II) ethanesulfonate, “CDCl₃” means deuterated chloroform, “MW” means microwave or molecular weight, “min” means minutes, “h” means hours, “rt” means room temperature, “quant” means quantitative, “n.t.” means not tested, “Cpd” means compound, “POCl₃” means phosphorus(V) oxychloride.

For key intermediates, as well as some final compounds, the absolute configuration of chiral centers (indicated as R and/or S) were established via comparison with samples of known configuration, or the use of analytical techniques suitable for the determination of absolute configuration, such as VCD (vibrational circular dichroism) or X-ray crystallography. When the absolute configuration at a chiral center is unknown, it is arbitrarily designated R*.

### EXAMPLES

### Example A

### Preparation of Intermediates

### Synthesis of 1H-indole-2-carbohydrazide 1A

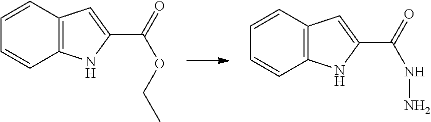

Hydrazine hydrate [7803-57-8] (13.66 mL, 0.28 mol) was added to a stirred solution of ethyl indole-2-carboxylate [3770-50-1] (5.2 g, 0.027 mol) in EtOH (40 mL) in a 100 ml round bottom flask. The mixture was stirred at 75° C. for 20 h. After that, the reaction mixture was cooled to 0° C. and the solids were filtered, washed with cold water and cold EtOH and then dried under vacuo at 50° C. overnight to yield 1H-indole-2-carbohydrazide 1A (4.20 g, 87%) as a white crystalline solid.

Structure analogs were synthesized using the same procedure.

| Reagent | Intermediate | Yield % | Number |
|---|---|---|---|
| 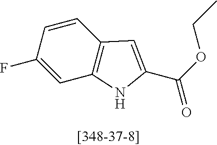 | 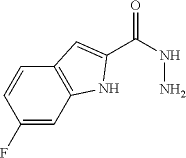 [SMILES: NNC(=O)c1cc2ccc(F)cc2[nH]1] | 65 | 2A |
| 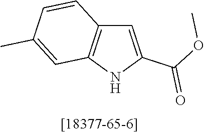 | 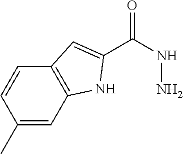 | 91 | 3A |
| 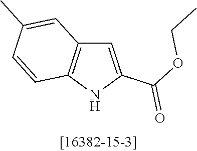 | 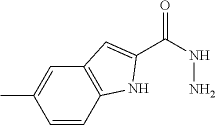 | 92 | 4A |
| 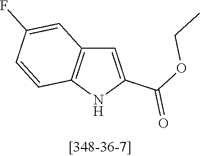 | 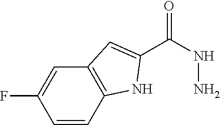 [SMILES: NNC(=O)c1cc2cc(F)ccc2[nH]1] | 90 | 5A |

### Synthesis of 4-methoxy-2H-[1,2,4]triazino[4,5-a]indol-1-one 1B

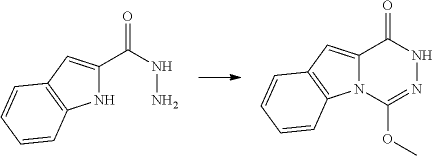 [SMILES: NNC(=O)c1cc2ccccc2[nH]1 ; COc1n[nH]c(=O)c2cc3ccccc3n12]

Aluminum isopropoxide [555-31-7] (0.116 g, 0.57 mmol) was added to a stirred mixture of 1H-indole-2-carbohydrazide [5055-39-0] 1A (1 g, 5.71 mmol) and tetramethyl orthocarbonate [1850-14-2] (1.14 mL, 8.56 mmol) in ACN (17.9 mL) at room temperature in a microwave vial. The mixture was stirred at 50° C. for 3 days. The reaction has stopped in the intermediate N-(dimethoxymethyleneamino)-1H-indole-2-carboxamide, so it is heated at 120° C. for 1 week. Then, the mixture was diluted with water and extracted with DCM. The organic layer was separated, dried (Na₂SO₄), filtered and the solvents evaporated in vacuo. The crude product was triturated with Et₂O to yield 4-methoxy-2H-[1,2,4]triazino[4,5-a]indol-1-one 1B (0.97 g, 72%) as a brown solid.

Structure analogs were synthesized using the same procedure.

| Reagent | Intermediate | Product | Yield % | Number |
|---|---|---|---|---|
| 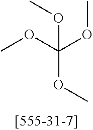 | 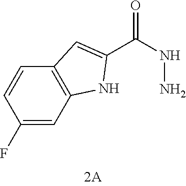 [SMILES: NNC(=O)c1cc2ccc(F)cc2[nH]1] | 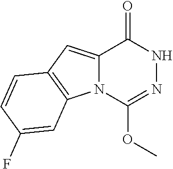 [SMILES: COc1n[nH]c(=O)c2cc3ccc(F)cc3n12] | 72 | 2B |
| 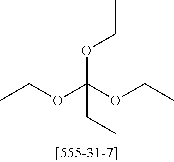 | 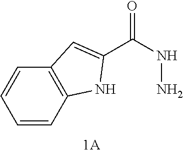 [SMILES: NNC(=O)c1cc2ccccc2[nH]1] | 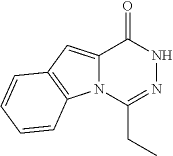 [SMILES: CCc1n[nH]c(=O)c2cc3ccccc3n12] | 39 | 3B |
| 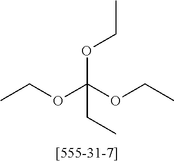 | 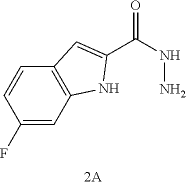 [SMILES: NNC(=O)c1cc2ccc(F)cc2[nH]1] | 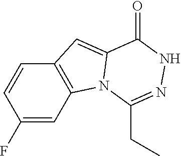 [SMILES: CCc1n[nH]c(=O)c2cc3ccc(F)cc3n12] | 95 | 4B |
| 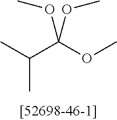 | 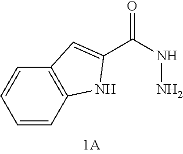 [SMILES: NNC(=O)c1cc2ccccc2[nH]1] | 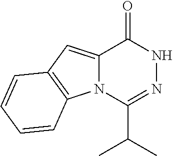 [SMILES: CC(C)c1n[nH]c(=O)c2cc3ccccc3n12] | 51 | 5B |
| 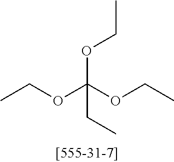 | 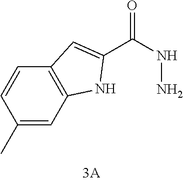 | 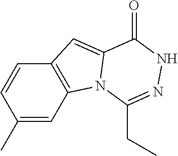 [SMILES: CCc1n[nH]c(=O)c2cc3ccc(C)cc3n12] | 75^{a} | 8B |
| 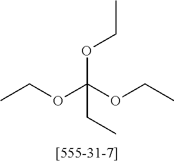 | 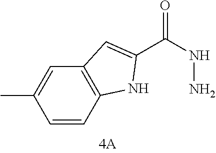 | 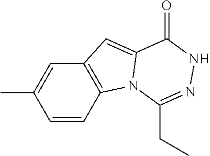 [SMILES: CCc1n[nH]c(=O)c2cc3cc(C)ccc3n12] | 51^{b} | 9B |
| 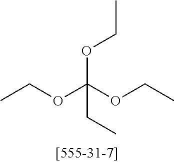 | 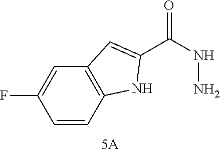 [SMILES: NNC(=O)c1cc2cc(F)ccc2[nH]1] | 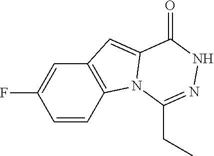 [SMILES: CCc1n[nH]c(=O)c2cc3cc(F)ccc3n12] | 16^{b} | 10B |
| 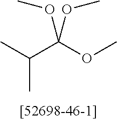 | 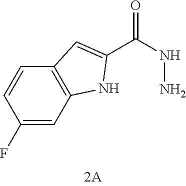 [SMILES: NNC(=O)c1cc2ccc(F)cc2[nH]1] | 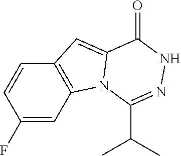 [SMILES: CC(C)c1n[nH]c(=O)c2cc3ccc(F)cc3n12] | 77^{c} | 11B |

aObtained as a 2:3 mixture of triazinone 8B and regioisomeric indolo-oxadiazole;
^{b}Crude product boiled in methanol (75 mL per l g of hydrazide used in the reaction), cooled to it and filtered to obtain pure triazinone product;
^{c}Obtained as a 88:12 mixture of triazinone 11B and regioisomeric indolo-oxadiazole.

### Synthesis of 10-chloro-2H-[1,2,4]triazino[4,5-a]indol-1-one 6B

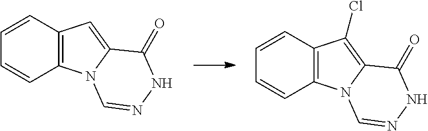 [SMILES: O=c1[nH]ncn2c1cc1ccccc12 ; O=c1[nH]ncn2c1c(Cl)c1ccccc12]

N-Chlorosuccinimide [128-09-6] (0.39 g, 2.97 mmol) was added portionwise to a solution of 2H-[1,2,4]triazino[4,5-a]indol-1-one [37574-73-5] (0.5 g, 2.70 mmol) in DMF (15 mL) and MeOH (1.5 mL). The mixture was heated in a closed pressure tube at 60° C. for 24h. The mixture was evaporated in vacuo and the crude product was boiled in ACN. The solid formed was filtered off to yield 10-chloro-2H-[1,2,4]triazino[4,5-a]indol-1-one 6B (498 mg, 84%) as a white solid.

### Synthesis of 4-bromo-10-chloro-2H-[1,2,4]triazino[4,5-a]indol-1-one 7B

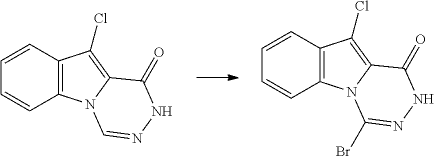 [SMILES: O=c1[nH]ncn2c1c(Cl)c1ccccc12 ; O=c1[nH]nc(Br)n2c1c(Cl)c1ccccc12]

Grinded K₂CO₃ [584-08-7] (1.57 g, 11.38 mmol) was added to a stirred suspension of 10-chloro-2H-[1,2,4]triazino[4,5-a]indol-1-one 6B (1 g, 4.55 mmol) in ACN (136 mL). The mixture was stirred for 15 min at rt, then benzyltrimethylammonium tribromide [111865-47-5] (2.31 g, 5.92 mmol) was added portionwise followed by the addition of MeOH (1.85 mL). The mixture was stirred at rt for 48 h. The mixture was diluted with water and extracted with DCM. The organic layer was separated, dried (MgSO4), filtered and evaporated in vacuo. The crude product was purified by flash column chromatography (silica, MeOH in DCM 0/100 to 2/98). The desired fractions were collected and concentrated in vacuo to yield 4-bromo-10-chloro-2H-[1,2,4]triazino[4,5-a]indol-1-one 7B (615 mg, 45%) as a white solid.

### Synthesis of methyl 2-(4-methoxy-1-oxo-[1,2,4]triazino[4,5-a]indol-2-yl)acetate 1C

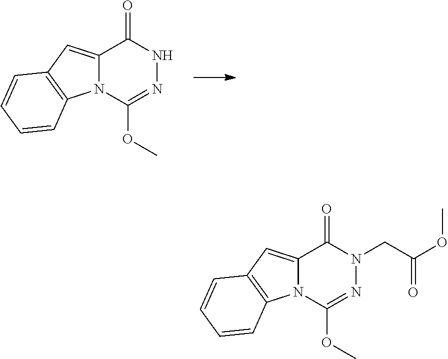 [SMILES: COc1n[nH]c(=O)c2cc3ccccc3n12 ; COC(=O)Cn1nc(OC)n2c(cc3ccccc32)c1=O]

A mixture of 4-methoxy-2H-[1,2,4]triazino[4,5-a]indol-1-one 1B (0.3 g, 1.28 mmol), methyl 2-chloroacetate [96-34-4] (0.16 mL, 1.92 mmol), 18-crown-6 [17455-13-9] (17 mg, 0.064 mmol), KI [7681-11-0] (26 mg, 0.15 mmol) and K₂CO₃ [584-08-7] (0.27 g, 1.92 mmol) in ACN (10 mL) was stirred at 80° C. overnight. The mixture was diluted with water was added and extracted with DCM, dried (Na₂SO₄) and concentrated in vacuo to afford methyl 2-(4-methoxy-1-oxo-[1,2,4]triazino[4,5-a]indol-2-yl)acetate 1C (0.36 g, 93%) as a brown solid. The product was used in next step without further purification.

Structure analogs were synthesized using the same procedure.

| reagent | reagent | Intermediate | Yield % | Number |
|---|---|---|---|---|
| 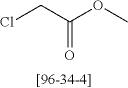 | 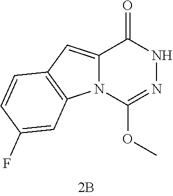 [SMILES: COc1n[nH]c(=O)c2cc3ccc(F)cc3n12] | 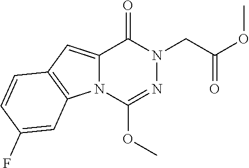 [SMILES: COC(=O)Cn1nc(OC)n2c(cc3ccc(F)cc32)c1=O] | 98 | 2C |
| 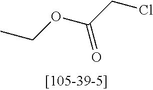 | 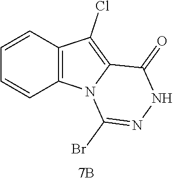 [SMILES: O=c1[nH]nc(Br)n2c1c(Cl)c1ccccc12] | 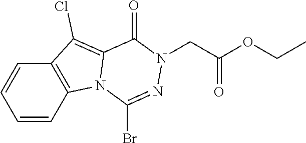 [SMILES: CCOC(=O)Cn1nc(Br)n2c(c(Cl)c3ccccc32)c1=O] | 77 | 3C |
| 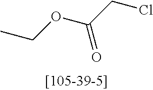 | 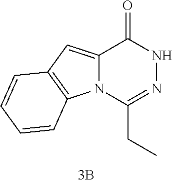 [SMILES: CCc1n[nH]c(=O)c2cc3ccccc3n12] | 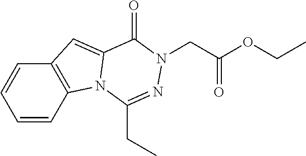 [SMILES: CCOC(=O)Cn1nc(CC)n2c(cc3ccccc32)c1=O] | 80 | 4C |
| 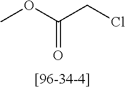 | 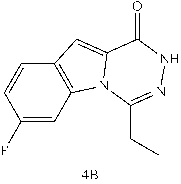 [SMILES: CCc1n[nH]c(=O)c2cc3ccc(F)cc3n12] | 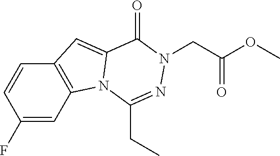 [SMILES: CCc1nn(CC(=O)OC)c(=O)c2cc3ccc(F)cc3n12] | 81 | 5C |
| 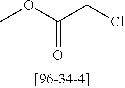 | 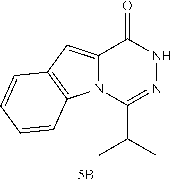 [SMILES: CC(C)c1n[nH]c(=O)c2cc3ccccc3n12] | 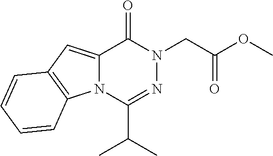 [SMILES: COC(=O)Cn1nc(C(C)C)n2c(cc3ccccc32)c1=O] | 98 | 6C |
| 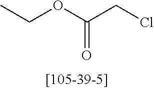 | 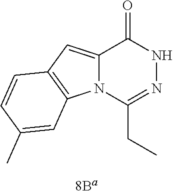 [SMILES: CCc1n[nH]c(=O)c2cc3ccc(C)cc3n12] | 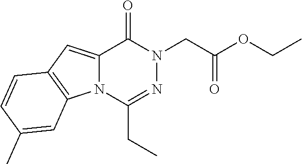 [SMILES: CCOC(=O)Cn1nc(CC)n2c(cc3ccc(C)cc32)c1=O] | 40^{b,c} | 28C |
| 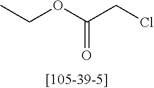 | 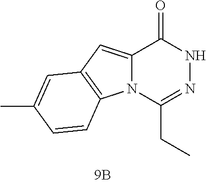 [SMILES: CCc1n[nH]c(=O)c2cc3cc(C)ccc3n12] | 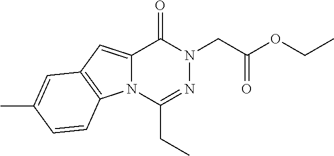 [SMILES: CCOC(=O)Cn1nc(CC)n2c(cc3cc(C)ccc32)c1=O] | 59^{b} | 29C |
| 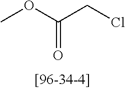 | 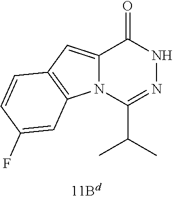 [SMILES: CC(C)c1n[nH]c(=O)c2cc3ccc(F)cc3n12] | 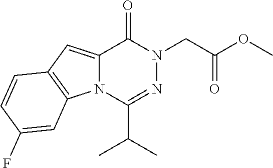 [SMILES: COC(=O)Cn1nc(C(C)C)n2c(cc3ccc(F)cc32)c1=O] | 100 | 31C |
| 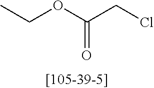 | 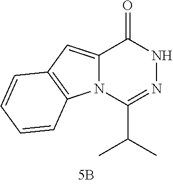 [SMILES: CC(C)c1n[nH]c(=O)c2cc3ccccc3n12] | 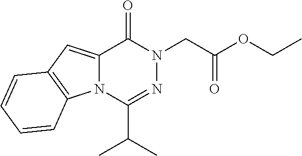 [SMILES: CCOC(=O)Cn1nc(C(C)C)n2c(cc3ccccc32)c1=O] | 91^{e} | 32C |
| 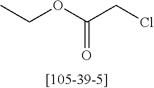 | 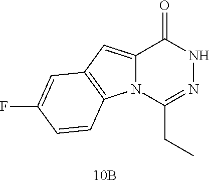 [SMILES: CCc1n[nH]c(=O)c2cc3cc(F)ccc3n12] | 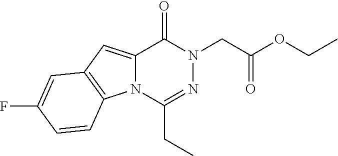 [SMILES: CCOC(=O)Cn1nc(CC)n2c(cc3cc(F)ccc32)c1=O] | 76 | 33C |

^{a}Ca. 2:3 mixture of 8B (triazinone) and regioisomeric indolo-oxadiazole;
^{b}Reacted for 7 h at 85° C.;
^{c}Purified by reverse phase preparative HPLC;
^{d}Ca. 88:12 mixture of 11B (triazinone) and regioisomeric indolo-oxadiazole;
^{e}Reacted for 3 h at 90° C.

### Synthesis of ethyl 2-(10-chloro-4-iodo-1-oxo-[1,2,4]triazino[4,5-a]indol-2-yl)acetate 7C

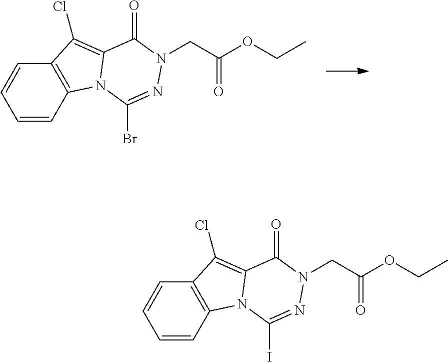 [SMILES: CCOC(=O)Cn1nc(Br)n2c(c(Cl)c3ccccc32)c1=O ; CCOC(=O)Cn1nc(I)n2c(c(Cl)c3ccccc32)c1=O]

CuI [7681-65-4] (15 mg, 0.078 mmol) was added to a stirred mixture of ethyl 2-(4-bromo-10-chloro-1-oxo-[1,2,4]triazino[4,5-a]indol-2-yl)acetate 3C (100 mg, 0.26 mmol), NaI [7681-82-5] (78 mg, 0.52 mmol), trans-N,N′-dimethylcyclohexane-1,2-diamine [67579-81-1] (11 mg, 0.078 mmol), in 1,4-dioxane (3.9 mL) and heated at 120° C. for 3 h. The mixture was evaporated in vacuo and the crude was purified by flash column chromatography (silica, DCM 100%). The desired fractions were collected and concentrated in vacuo to yield ethyl 2-(10-chloro-4-iodo-1-oxo-[1,2,4]triazino[4,5-a]indol-2-yl)acetate 7C (50 mg, 45%) as a white solid.

### Synthesis of ethyl 2-[10-chloro-1-oxo-4-(trifluoromethyl)-[1,2,4]triazino[4,5-a]indol-2-yl]acetate 8C

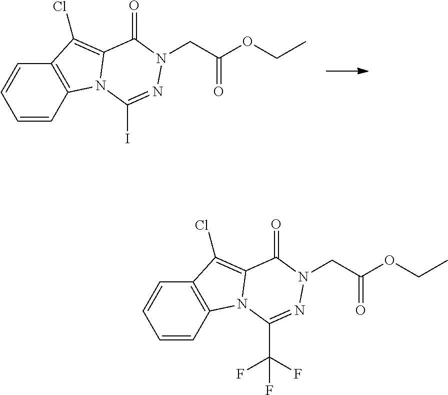 [SMILES: CCOC(=O)Cn1nc(I)n2c(c(Cl)c3ccccc32)c1=O ; CCOC(=O)Cn1nc(C(F)(F)F)n2c(c(Cl)c3ccccc32)c1=O]

A solution of ethyl 2-[10-chloro-1-oxo-4-(trifluoromethyl)-[1,2,4]triazino[4,5-a]indol-2-yl]acetate 7C (50 mg, 0.12 mmol) in anhydrous DMF (1.26 mL) was flushed with N₂ for a few minutes. Then CuI [7681-65-4] (66 mg, 0.35 mmol) was added followed by methyl 2,2-difluoro-2-(fluorosulfonyl) acetate [680-15-9] (67 mg, 0.35 mmol). The reaction was heated at 100° C. in a sealed tube for 2 h. The mixture was filtered through celite and then diluted with MeOH. The product was purified by RP HPLC (stationary phase: XBridge Prep C18 OBD-10 μm, 30×150 mm, mobile phase: 0.25% NH₄HCO₃ solution in water, CH₃CN). The pure fractions were evaporated in vacuo yielding ethyl 2-[10-chloro-1-oxo-4-(trifluoromethyl)-[1,2,4]triazino[4,5-a]indol-2-yl]acetate 8C (25 mg, 58%) as a white solid.

### Synthesis of ethyl 2-[1-oxo-4-(trifluoromethyl)-[1,2,4]triazino[4,5-a]indol-2-yl]acetate 9C

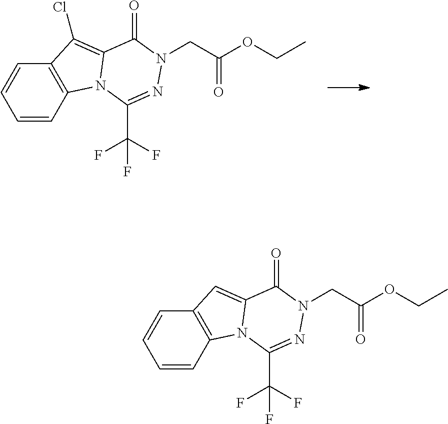 [SMILES: CCOC(=O)Cn1nc(C(F)(F)F)n2c(c(Cl)c3ccccc32)c1=O ; CCOC(=O)Cn1nc(C(F)(F)F)n2c(cc3ccccc32)c1=O]

Palladium on activated charcoal degussa type [7440-05-3] (10 mg, 0.009 mmol, 10 wt %) was added to a stirred mixture of ethyl 2-[10-chloro-1-oxo-4-(trifluoromethyl)-[1,2,4]triazino[4,5-a]indol-2-yl]acetate 8C (20 mg, 0.05 mol) and TEA [121-44-8] (11 μL, 0.08 mmol) in EtOH (10 mL) under N₂ and then hydrogenated at rt for 2 h. The mixture was filtered through celite and the filtrate was evaporated in vacuo to yield ethyl 2-[1-oxo-4-(trifluoromethyl)-[1,2,4]triazino[4,5-a]indol-2-yl]acetate 9C (20 mg, 100%). The product was used in next step without further purification.

### Synthesis of methyl 2-(1,4-dioxo-3H-[1,2,4]triazino[4,5-a]indol-2-yl)acetate 10C

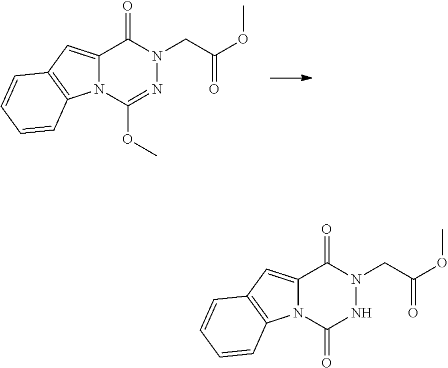 [SMILES: COC(=O)Cn1nc(OC)n2c(cc3ccccc32)c1=O ; COC(=O)Cn1[nH]c(=O)n2c(cc3ccccc32)c1=O]

Chlorotrimethylsilane [75-77-4] (0.28 g, 2.62 mmol) and NaI [7681-82-5] (0.39 g, 2.63 mmol) were added to a solution of methyl 2-(4-methoxy-1-oxo-[1,2,4]triazino[4,5-a]indol-2-yl)acetate 1C (0.36 g, 1.19 mmol) in ACN (6.2 mL). The reaction was stirred at 80° C. for 1 h. Water was added and the precipitate formed was filtered, washed with water and dried under vacuo to afford methyl 2-(1,4-dioxo-3H-[1,2,4]triazino[4,5-a]indol-2-yl)acetate 10C (0.28 g, 86%) as a yellowish white solid. The product was used in next step without further purification.

Structure analog was synthesized using the same procedure:

| | | Yield | |
|---|---|---|---|
| Intermediate | Product | % | Number |
| 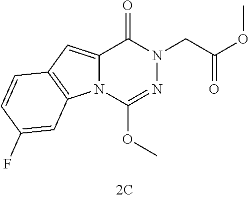 [SMILES: COC(=O)Cn1nc(OC)n2c(cc3ccc(F)cc32)c1=O] | 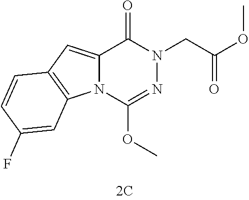 [SMILES: COC(=O)Cn1nc(OC)n2c(cc3ccc(F)cc32)c1=O] | 73 | 11C |

### Synthesis of methyl 2-[1-oxo-4-(trifluoromethylsulfonyloxy)-[1,2,4]triazino[4,5-a]indol-2-yl]acetate 12C

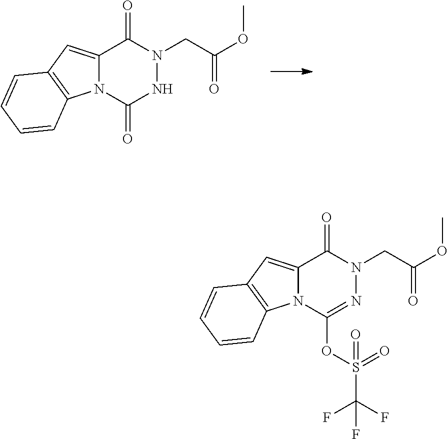 [SMILES: COC(=O)Cn1[nH]c(=O)n2c(cc3ccccc32)c1=O ; COC(=O)Cn1nc(OS(=O)(=O)C(F)(F)F)n2c(cc3ccccc32)c1=O]

Methyl 2-(1,4-dioxo-3H-[1,2,4]triazino[4,5-a]indol-2-yl)acetate 10C (100 mg, 0.37 mmol) was dissolved in DCM (9.4 mL) and TBA [121-44-8] (0.10 mL, 0.73 mmol) and cooled down to 0° C. The vial was purged with N₂, and trifluoromethanesulfonic anhydride [358-23-6] (0.12 mL, 0.73 mmol) was added dropwise. The mixture was allowed to warm to rt and stirred overnight. The mixture was diluted with water and extracted with DCM. The combined organic layers were concentrated in vacuo. The crude product was dissolved in a small amount of DCM, filtered through a silica plug (2 g) and eluted with additional DCM. The collected colourless solution was concentrated in vacuo to afford methyl 2-[1-oxo-4-(trifluoromethylsulfonyloxy)-[1,2,4]triazino[4,5-a]indol-2-yl]acetate 12C (97 mg, 65%) as a white crystalline solid.

### Synthesis of methyl 2-[4-(dimethylamino)-1-oxo-[1,2,4]triazino[4,5-a]indol-2-yl]acetate 13C

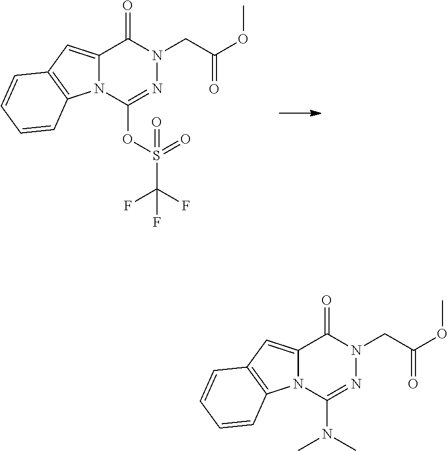 [SMILES: COC(=O)Cn1nc(OS(=O)(=O)C(F)(F)F)n2c(cc3ccccc32)c1=O ; COC(=O)Cn1nc(N(C)C)n2c(cc3ccccc32)c1=O]

Methyl 2-[1-oxo-4-(trifluoromethylsulfonyloxy)-[1,2,4]triazino[4,5-a]indol-2-yl]acetate 12C (265 mg, 0.65 mmol) and dimethylamine [75-09-2] (1.63 mL, 3.27 mmol, 2N) were mixed in a vial. The vial was purged with N₂ and stirred at rt for 10 min. The mixture was diluted with water and extracted with DCM. The organic layer was separated, and dried (Na₂SO₄), filtered and the solvents concentrated in vacuo to afford methyl 2-[4-(dimethylamino)-1-oxo-[1,2,4]triazino[4,5-a]indol-2-yl]acetate 13C (99 mg, 50%) as white solid. The product was used in next step without further purification.

### Synthesis of methyl 2(4-chloro-1-oxo-1,2,4 triazino[4,5-a]indol-2-1 acetate 14C

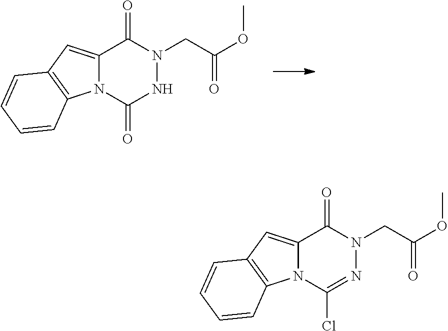 [SMILES: COC(=O)Cn1[nH]c(=O)n2c(cc3ccccc32)c1=O ; COC(=O)Cn1nc(Cl)n2c(cc3ccccc32)c1=O]

Methyl 2-(1,4-dioxo-3H-[1,2,4]triazino[4,5-a]indol-2-yl)acetate 10C (500 mg, 1.83 mmol) and POCl₃ [10025-87-3] (5 mL, 53.80 mmol) were mixed in a sealed vial and heated at 125° C. for 4h. The mixture was evaporated in vacuo. The crude product was diluted with a saturated aqueous solution of NaHCO₃ and extracted with DCM. The combined organic layers were washed with water, dried (Na₂SO₄) and concentrated in vacuo. The resulting solid was washed with ACN (5 mL), filtered and dried under vacuo at 50° C. for 2 h to afford methyl 2-(4-chloro-1-oxo-[1,2,4]triazino[4,5-a]indol-2-yl)acetate 14C (400 mg, 75%) as cream solid.

Structure analog was synthesized using the same procedure:

| | | Yield | |
|---|---|---|---|
| Intermediate | Product | % | Number |
| 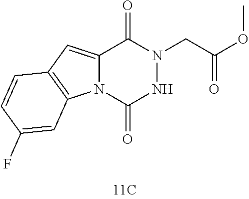 [SMILES: COC(=O)Cn1[nH]c(=O)n2c(cc3ccc(F)cc32)c1=O] | 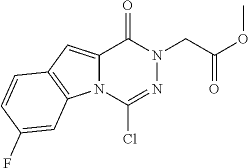 [SMILES: COC(=O)Cn1nc(Cl)n2c(cc3ccc(F)cc32)c1=O] | 90 | 15C |

### Synthesis of methyl 2-(1-oxo-4-phenyl-[1,2,4]triazino[4,5-a]indol-2-yl)acetate 16C and methyl 2-(1-oxo-[1,2,4]triazino[4,5-a]indol-2-yl)acetate 17C

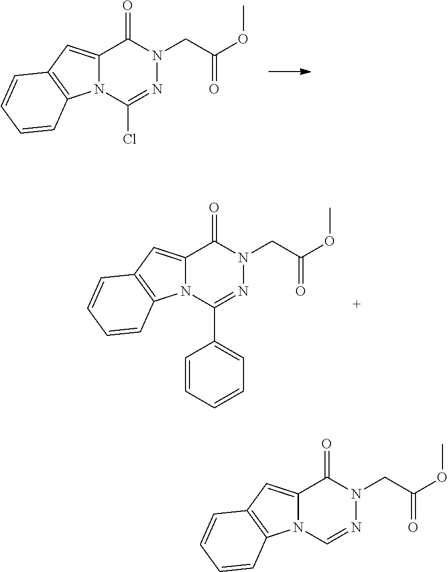 [SMILES: COC(=O)Cn1nc(Cl)n2c(cc3ccccc32)c1=O ; COC(=O)Cn1nc(-c2ccccc2)n2c(cc3ccccc32)c1=O ; COC(=O)Cn1ncn2c(cc3ccccc32)c1=O]

Bis[tris(tert-butyl)phosphine]palladium [53199-31-8](35 mg, 0.07 mmol) was added to a stirred solution of methyl 2-(4-chloro-1-oxo-[1,2,4]triazino[4,5-a]indol-2-yl)acetate 14C (100 mg, 0.34 mmol), phenylboronic acid [98-80-6] (50 mg, 0.41 mmol) and TEA [121-44-8] (0.24 mL, 1.71 mmol) in THF (5 mL) in a sealed tube and under N₂. The reaction was stirred at 120° C. for 16 h. The cooled reaction was concentrated in vacuo and purified by RP HPLC (stationary phase: C18 XBridge 30×100 mm 5 μm), mobile phase: gradient from 80% NH₄HCO₃ 0.25% solution in water, 20% CH₃CN to 0% NH₄HCO₃ 0.25% solution in water, 100% CH₃CN) to afford methyl 2-(1-oxo-4-phenyl-[1,2,4]triazino[4,5-a]indol-2-yl)acetate 16C (25 mg, 24%) and methyl 2-(1-oxo-[1,2,4]triazino[4,5-a]indol-2-yl)acetate 17C (31 mg, 40%).

### Synthesis of methyl 2-(7-fluoro-1-oxo-4-phenyl-[1,2,4]triazino[4,5-a]indol-2-yl)acetate 18C and methyl 2-(7-fluoro-1-oxo-[1,2,4]triazino[4,5-a]indol-2-yl)acetate 19C

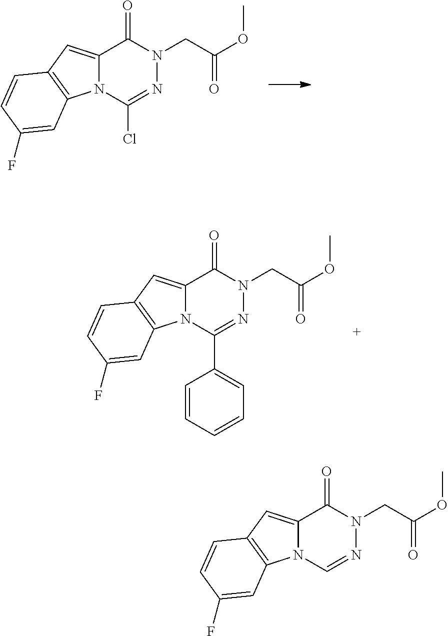 [SMILES: COC(=O)Cn1nc(Cl)n2c(cc3ccc(F)cc32)c1=O ; COC(=O)Cn1nc(-c2ccccc2)n2c(cc3ccc(F)cc32)c1=O ; COC(=O)Cn1ncn2c(cc3ccc(F)cc32)c1=O]

XPhos Pd G3 [1445085-55-1](27 mg, 0.03 mmol) was added to a stirred solution of methyl 2-(4-chloro-7-fluoro-1-oxo-[1,2,4]triazino[4,5-a]indol-2-yl)acetate 15C (100 mg, 0.32 mmol), phenylboronic acid [98-80-6] (78 mg, 0.64 mmol) and TEA [121-44-8] (0.09 mL, 0.64 mmol) in THF (25 mL) in a sealed tube and under N₂. The reaction was stirred at 120° C. for 16 h. The cooled reaction was concentrated in vacuo and purified by RP HPLC (stationary phase: C18 XBridge 30×100 mm 5 μm), mobile phase: gradient from 80% NH₄HCO₃ 0.25% solution in water, 20% CH₃CN to 0% NH₄HCO₃ 0.25% solution in water, 100% CH₃CN) to afford methyl 2-(7-fluoro-1-oxo-4-phenyl-[1,2,4]triazino[4,5-a]indol-2-yl)acetate 18C (54 mg, 48%) and methyl 2-(7-fluoro-1-oxo-[1,2,4]triazino[4,5-a]indol-2-yl)acetate 19C (20 mg, 22%) both as cream solids.

### Synthesis of methyl 2-[4-(1-ethoxyvinyl)-1-oxo-[1,2,4]triazino[4,5-a]indol-2-yl]acetate 20C

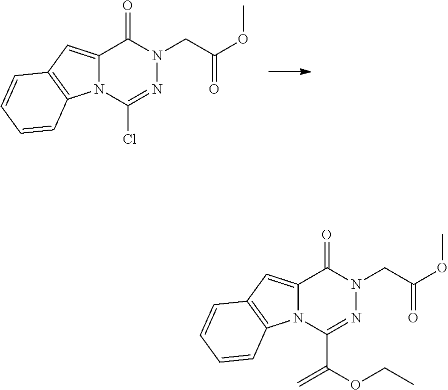 [SMILES: COC(=O)Cn1nc(Cl)n2c(cc3ccccc32)c1=O ; C=C(OCC)c1nn(CC(=O)OC)c(=O)c2cc3ccccc3n12]

Bis[tris(tert-butyl)phosphine]palladium [53199-31-8] (100 mg, 0.19 mmol) was added to a stirred solution of methyl 2-(4-chloro-1-oxo-[1,2,4]triazino[4,5-a]indol-2-yl)acetate 14C (200 mg, 0.68 mmol) and 1-ethoxy-1-(tributylstannyl)ethylene (0.47 mL, 1.38 mmol) in THF (12 mL). The reaction was heated at 90° C. for 3 h. The cooled mixture was concentrated in vacuo and purified by flash column chromatography (silica; EtOAc in heptane 0/100 to 40/60). The desired fractions were collected and concentrated in vacuo to yield methyl 2-[4-(1-ethoxyvinyl)-1-oxo-[1,2,4]triazino[4,5-a]indol-2-yl]acetate 20C (145 mg, 65%) as a white solid.

Structure analogs were synthesized using the same procedure:

| | | | Yield | |
|---|---|---|---|---|
| reagent | Intermediate | Product | % | Number |
| 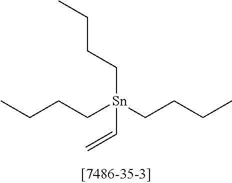 | 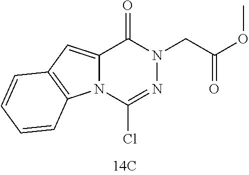 [SMILES: COC(=O)Cn1nc(Cl)n2c(cc3ccccc32)c1=O] | 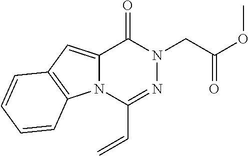 [SMILES: C=Cc1nn(CC(=O)OC)c(=O)c2cc3ccccc3n12] | 78 | 21C |
| 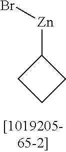 | 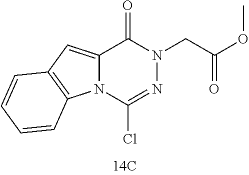 [SMILES: COC(=O)Cn1nc(Cl)n2c(cc3ccccc32)c1=O] | 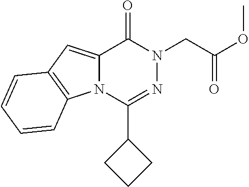 [SMILES: COC(=O)Cn1nc(C2CCC2)n2c(cc3ccccc32)c1=O] | 37 | 22C |
| 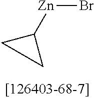 | 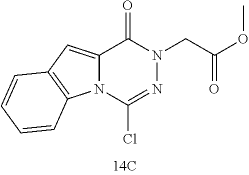 [SMILES: COC(=O)Cn1nc(Cl)n2c(cc3ccccc32)c1=O] | 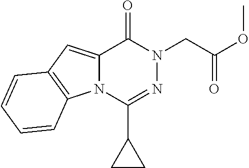 [SMILES: COC(=O)Cn1nc(C2CC2)n2c(cc3ccccc32)c1=O] | 73 | 23C |

### Synthesis of methyl 2-(4-formyl-1-oxo-[1,2,4]triazino[4,5-a]indol-2-yl)acetate 24C

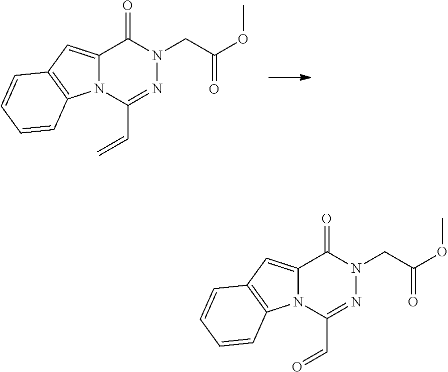 [SMILES: C=Cc1nn(CC(=O)OC)c(=O)c2cc3ccccc3n12 ; COC(=O)Cn1nc(C=O)n2c(cc3ccccc32)c1=O]

Sodium metaperiodate [7790-28-5] (2.5 g, 12 mmol) was added to a stirred mixture of methyl 2-(1-oxo-4-vinyl-[1,2,4]triazino[4,5-a]indol-2-yl)acetate 21C (0.85 g, 3 mmol), osmium tetroxide [20816-12-0] (2.69 mL, 0.06 mmol, 0.89 M) and N-methylmorpholine N-oxide [7529-22-8] (1.05 g, 9 mmol), in 1,4-dioxane (43 mL) and water (15 mL). The reaction was stirred at rt for 24 h. Then, a saturated aqueous solution of NaHCO₃ was added and the product was extracted in EtOAc. The combined organic layers were washed with a 10% aqueous solution of sodium bisulfite, dried (Na₂SO₄), filtered and the solvents concentrated in vacuo to afford methyl 2-(4-formyl-1-oxo-[1,2,4]triazino[4,5-a]indol-2-yl)acetate 24C (0.55 g, 64%) as oil which that solidified upon standing.

### Synthesis of methyl 2-[4-(difluoromethyl)-1-oxo-[1,2,4]triazino[4,5-a]indol-2-yl]acetate 25C

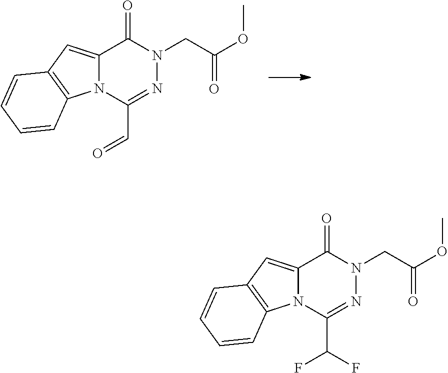 [SMILES: COC(=O)Cn1nc(C=O)n2c(cc3ccccc32)c1=O ; COC(=O)Cn1nc(C(F)F)n2c(cc3ccccc32)c1=O]

Bis(2-methoxyethyl) aminosulfur trifluoride [202289-38-1] (0.9 mL, 1.77 mmol) was added to a stirred solution of methyl 2-(4-formyl-1-oxo-[1,2,4]triazino[4,5-a]indol-2-yl)acetate 24C (0.230 g, 0.81 mmol) in DCM (8 mL) at 0° C. The reaction was stirred at 0° C. for 1 h and at rt for 2 h. A saturated aqueous solution of NaHCO₃ was added and the reaction was stirred at rt for 30 min. Then, the mixture was extracted with DCM and the combined organic layers were washed with water, dried (Na₂SO₄), filtered and the solvents concentrated in vacuo. The crude product was purified by flash column chromatography (silica; MeOH in DCM 0/100 to 1/99). The desired fractions were collected and concentrated in vacuo to yield methyl 2-[4-(difluoromethyl)-1-oxo-[1,2,4]triazino[4,5-a]indol-2-yl]acetate 25C (140 mg, 56%) as a white solid.

### Synthesis of methyl 2-(4-acetyl-1-oxo-[1,2,4]triazino[4,5-a]indol-2-yl)acetate 26C

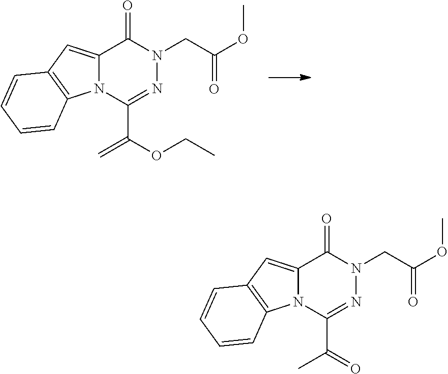 [SMILES: C=C(OCC)c1nn(CC(=O)OC)c(=O)c2cc3ccccc3n12 ; COC(=O)Cn1nc(C(C)=O)n2c(cc3ccccc32)c1=O]

A 6M aqueous solution of HCl [7647-01-0] (5 mL, 30 mmol) was added to a stirred solution of methyl 2-[4-(1-ethoxyvinyl)-1-oxo-[1,2,4]triazino[4,5-a]indol-2-yl]acetate 20C (250 mg, 0.76 mmol) in THF (10 mL).The reaction was stirred at rt for 16 h. Water was added and the product was extracted in EtOAc. The combined organic layers were dried (Na₂SO₄), filtered and the solvents concentrated in vacuo to afford methyl 2-(4-acetyl-1-oxo-[1,2,4]triazino[4,5-a]indol-2-yl)acetate 26C (190 mg, 83%) as cream solid.

### Synthesis of methyl 2-[4-(1,1-difluoroethyl)-1-oxo-[1,2,4]triazino[4,5-a]indol-2-yl]acetate 27C

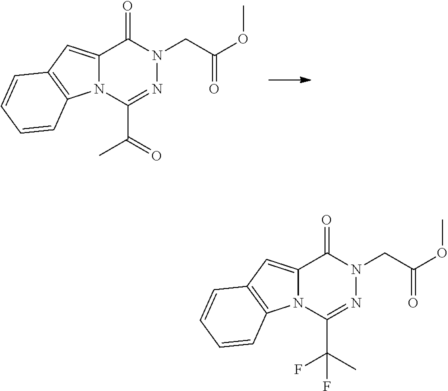 [SMILES: COC(=O)Cn1nc(C(C)=O)n2c(cc3ccccc32)c1=O ; COC(=O)Cn1nc(C(C)(F)F)n2c(cc3ccccc32)c1=O]

(Diethylamino)sulfur trifluoride [38078-09-0] (1.55 mL, 1.55 mmol, 1M) was added to a stirred solution of methyl 2-(4-acetyl-1-oxo-[1,2,4]triazino[4,5-a]indol-2-yl)acetate 26C (150 mg, 0.50 mmol) in DCM (25 mL) at 0° C. The reaction was stirred at rt for 72 h. The mixture was cooled at 0° C. and a saturated aqueous solution of NaHCO₃ was added dropwise. The mixture was stirred at rt for 30 min and then it was extracted with DCM. The combined organic layers were dried (Na₂SO₄), filtered and the solvents concentrated in vacuo. The crude product was purified by flash column chromatography (silica; MeOH in DCM 0/100 to 2/98). The desired fractions were collected and concentrated in vacuo to methyl 2-[4-(1,1-difluoroethyl)-1-oxo-[1,2,4]triazino[4,5-a]indol-2-yl]acetate 27C (100 mg, 62%) as a white solid.

### Synthesis of methyl 2-(4-(dimethylamino)-7-fluoro-1-oxo-[1,2,4]triazino[4,5-a]indol-2(1H)-yl)acetate 30C

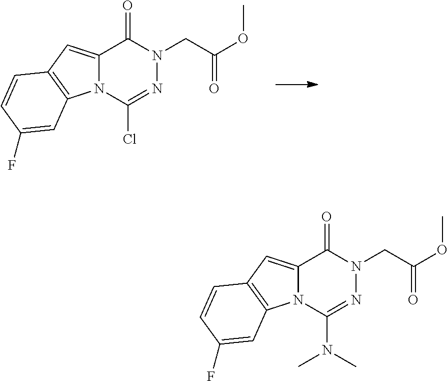 [SMILES: COC(=O)Cn1nc(Cl)n2c(cc3ccc(F)cc32)c1=O ; COC(=O)Cn1nc(N(C)C)n2c(cc3ccc(F)cc32)c1=O]

A solution of dimethylamine [124-40-3] (2M in THF, 2.8 mL, 5.6 mmol) was added to a stirred solution of methyl 2-(4-chloro-1-oxo-[1,2,4]triazino[4,5-a]indol-2-yl)acetate 15C (350 mg, 1.13 mmol) in acetonitrile (5 mL) at rt. The mixture was then stirred and heated at 120° C. for 20 min under MW irradiation. It was concentrated in vacuo to yield crude methyl 2-(4-(dimethylamino)-7-fluoro-1-oxo-[1,2,4]triazino[4,5-a]indol-2(1H)-yl)acetate 30C (450 mg, quantitative) as a brown solid that was used without further purification.

### Synthesis of 2-(4-cyano-1-oxo-[1,2,4]triazino[4,5-a]indol-2-yl)acetic acid 1D

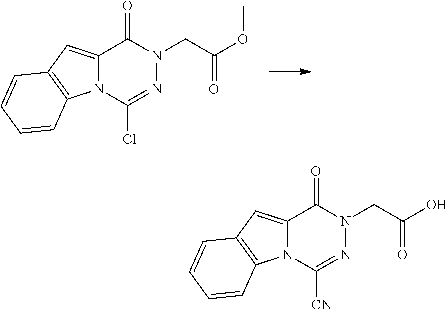 [SMILES: COC(=O)Cn1nc(Cl)n2c(cc3ccccc32)c1=O ; N#Cc1nn(CC(=O)O)c(=O)c2cc3ccccc3n12]

Sodium cyanide [143-33-9] (50 mg, 1.03 mmol) and 1,4-diazabicyclo[2.2.2]octane [280-57-9] (58 mg, 0.51 mmol) were added to a stirred solution of methyl 2-(4-chloro-1-oxo-[1,2,4]triazino[4,5-a]indol-2-yl)acetate 14C (100 mg, 0.34 mmol) in DMSO (3 mL). The mixture was stirred at 120° C. for 6 h. The mixture was diluted with water and extracted in EtOAc. The organic layer was separated, dried (Na₂SO₄), filtered and the solvents concentrated in vacuo to afford 2-(4-cyano-1-oxo-[1,2,4]triazino[4,5-a]indol-2-yl)acetic acid 1D (75 mg, 82%) as cream solid.

### Synthesis of 2-(4-oxazol-2-yl-1-oxo-[1,2,4]triazino[4,5-a]indol-2-yl)acetic acid 2D

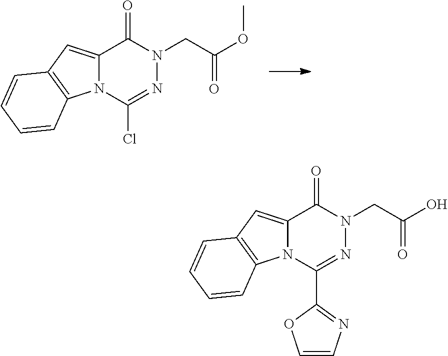 [SMILES: COC(=O)Cn1nc(Cl)n2c(cc3ccccc32)c1=O ; O=C(O)Cn1nc(-c2ncco2)n2c(cc3ccccc32)c1=O]

Bis[tris(tert-butyl)phosphine]palladium [53199-31-8] (42 mg, 0.08 mmol) was added to a stirred solution of methyl 2-(4-chloro-1-oxo-[1,2,4]triazino[4,5-a]indol-2-yl)acetate 14C (120 mg, 0.41 mmol), 2-(tributylstannyl)oxazole [145214-05-7] (368 mg, 1.03 mmol) and TEA [121-44-8] (0.14 mL, 1.03 mmol in THF (6 mL). The mixture was heated at 90° C. for 3 h. The cooled mixture was concentrated in vacuo and purified by flash column chromatography (silica; MeOH in DCM 0/100 to 2/98). The desired fractions were collected and concentrated in vacuo to yield 2-(4-oxazol-2-yl-1-oxo-[1,2,4]triazino[4,5-a]indol-2-yl)acetic acid 2D (75 mg, 59%) as a white solid.

### Synthesis of 2-(4-chloro-1-oxo-[1,2,4]triazino[4,5-a]indol-2-yl)acetic acid 3D. (Method A)

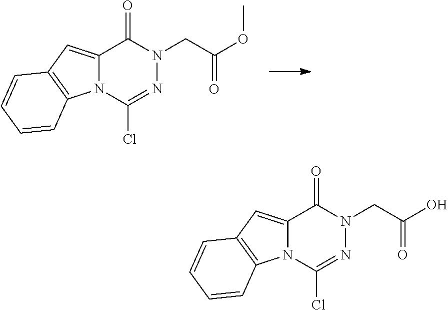 [SMILES: COC(=O)Cn1nc(Cl)n2c(cc3ccccc32)c1=O ; O=C(O)Cn1nc(Cl)n2c(cc3ccccc32)c1=O]

A 37% aqueous solution of HCl [7647-01-0] (3.5 mL, 41.91 mmol) was added to methyl 2-(4-chloro-1-oxo-[1,2,4]triazino[4,5-a]indol-2-yl)acetate 14C (350 mg, 1.2 mmol) in a sealed tube and the mixture was stirred at 120° C. for 16 hours. The mixture was cooled and concentrated in vacuo to afford 2-(4-chloro-1-oxo-[1,2,4]triazino[4,5-a]indol-2-yl)acetic acid 3D (340 mg, 80%) as cream solid.

### Synthesis of 2-[4-(1-ethoxyvinyl)-1-oxo-[1,2,4]triazino[4,5-a]indol-2-yl]acetic 4D. (Method B)

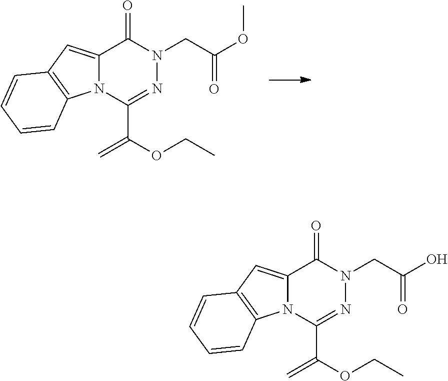 [SMILES: C=C(OCC)c1nn(CC(=O)OC)c(=O)c2cc3ccccc3n12 ; C=C(OCC)c1nn(CC(=O)O)c(=O)c2cc3ccccc3n12]

A 1N aqueous solution of NaOH [1310-73-2] (10 mL, 10 mmol) was added to a stirred solution of methyl 2-[4-(1-ethoxyvinyl)-1-oxo-[1,2,4]triazino[4,5-a]indol-2-yl]acetate 20C (502 mg, 1.53 mmol) in MeOH (5 mL). The mixture was stirred at 90° C. for 4 h and then acidified with a 1N aqueous solution of HCl until pH=2. The mixture was extracted in EtOAc and the organic layer was separated, dried (MgSO4), filtered and the solvents evaporated in vacuo to yield 2-[4-(1-ethoxyvinyl)-1-oxo-[1,2,4]triazino[4,5-a]indol-2-yl]acetic acid 4D (402 mg, 84%) as a white solid.

### Synthesis of 2-(4-ethyl-7-methyl-1-oxo-[1,2,4]triazino[4,5-a]indol-2(1H)-yl)acetic acid 5D. (Method C)

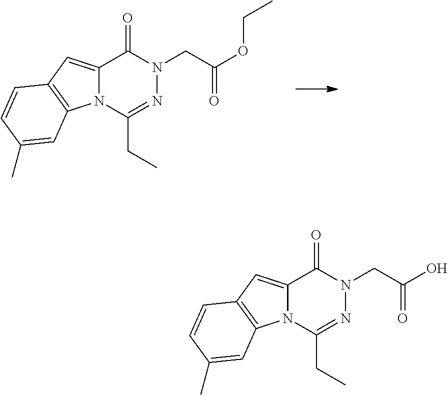 [SMILES: CCOC(=O)Cn1nc(CC)n2c(cc3ccc(C)cc32)c1=O ; CCc1nn(CC(=O)O)c(=O)c2cc3ccc(C)cc3n12]

Ester 28C (1 g, 3.19 mmol) was placed in a screw-cap vial equipped with a magnetic stir bar and dissolved in THF (11.7 mL), stirred vigorously at r.t. and an aqueous solution of LiOH [1310-65-2] (229.3 mg, 9.57 mmol) in DI water (15.8 mL) was added. The mixture was stirred vigorously at rt for 2 h. It was then acidified by addition of 1M aqueous HCl until pH 0-1 and the solvent was partially evaporated on a rotary evaporator. More DI water was added and the solids were filtered and dried under vacuum to obtain 2-(4-ethyl-7-methyl-1-oxo-[1,2,4]triazino[4,5-a]indol-2(1H)-yl)acetic acid 5D (525.5 mg, yield 58%) as a solid.

### Synthesis of lithium 2-(4-(dimethylamino)-7-fluoro-1-oxo-[1,2,4]triazino[4,5-a]indol-2(1H)-yl)acetate 6D. (Method D)

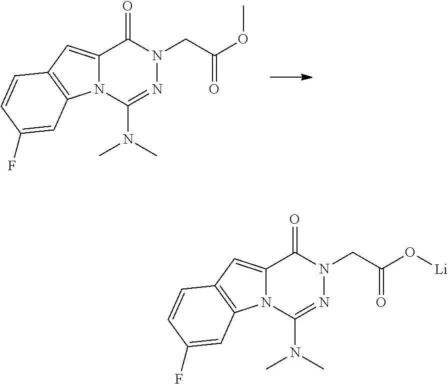

Ester 30C (400 mg, 1.18 mmol) was suspended in a mixture of THF (6 mL) and DI water (2 mL), the suspension stirred vigorously at r.t. and solid LiOH [1310-65-2] (113 mg, 4.73 mmol) was added. The mixture was stirred vigorously at rt 50° C. for 18 h. The volatiles were removed in vacuo on a rotary evaporator and the obtained solid (500 mg, quantitative) was dried in a desiccator and used without further purification.

Structure analogs were synthesized using the same procedures (Methods A, B, C or D):

| Meth- | | | Yield | |
|---|---|---|---|---|
| od | Intermediate | Product | % | Number |
| B | 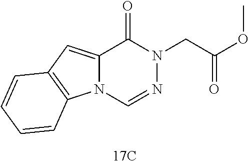 [SMILES: COC(=O)Cn1ncn2c(cc3ccccc32)c1=O] | 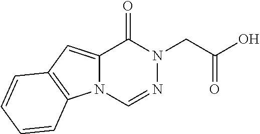 [SMILES: O=C(O)Cn1ncn2c(cc3ccccc32)c1=O] | 75 | 7D |
| B | 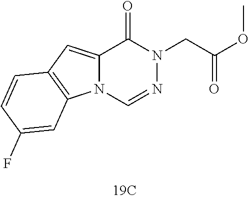 [SMILES: COC(=O)Cn1ncn2c(cc3ccc(F)cc32)c1=O] | 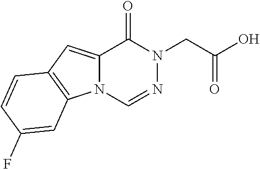 [SMILES: O=C(O)Cn1ncn2c(cc3ccc(F)cc32)c1=O] | 98 | 8D |
| B | 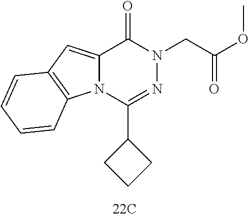 [SMILES: COC(=O)Cn1nc(C2CCC2)n2c(cc3ccccc32)c1=O] | 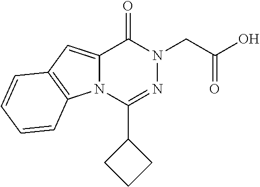 [SMILES: O=C(O)Cn1nc(C2CCC2)n2c(cc3ccccc32)c1=O] | 97 | 9D |
| A | 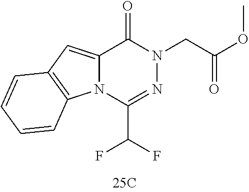 [SMILES: COC(=O)Cn1nc(C(F)F)n2c(cc3ccccc32)c1=O] | 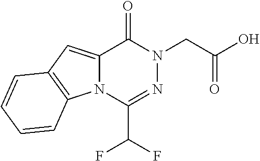 [SMILES: O=C(O)Cn1nc(C(F)F)n2c(cc3ccccc32)c1=O] | 82 | 10D |
| B | 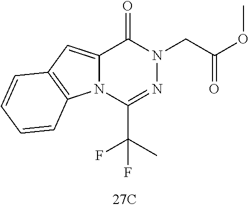 [SMILES: COC(=O)Cn1nc(C(C)(F)F)n2c(cc3ccccc32)c1=O] | 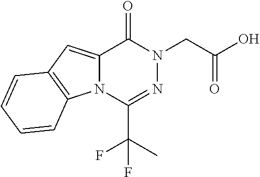 [SMILES: CC(F)(F)c1nn(CC(=O)O)c(=O)c2cc3ccccc3n12] | 84 | 11D |
| B | 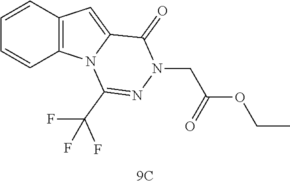 [SMILES: CCOC(=O)Cn1nc(C(F)(F)F)n2c(cc3ccccc32)c1=O] | 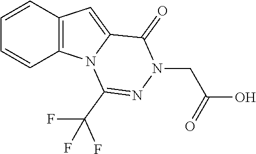 [SMILES: O=C(O)Cn1nc(C(F)(F)F)n2c(cc3ccccc32)c1=O] | 98 | 12D |
| B | 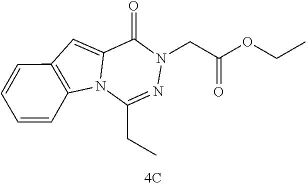 [SMILES: CCOC(=O)Cn1nc(CC)n2c(cc3ccccc32)c1=O] | 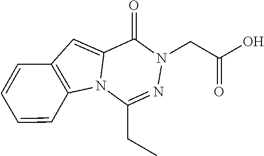 [SMILES: CCc1nn(CC(=O)O)c(=O)c2cc3ccccc3n12] | 80 | 13D |
| B | 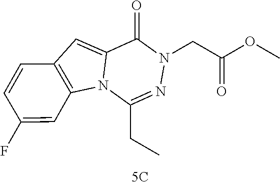 [SMILES: CCc1nn(CC(=O)OC)c(=O)c2cc3ccc(F)cc3n12] | 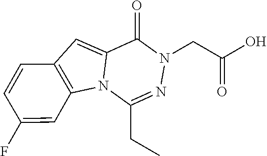 [SMILES: CCc1nn(CC(=O)O)c(=O)c2cc3ccc(F)cc3n12] | 85 | 14D |
| B | 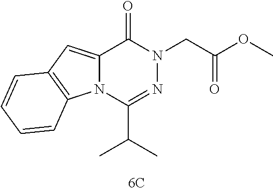 [SMILES: COC(=O)Cn1nc(C(C)C)n2c(cc3ccccc32)c1=O] | 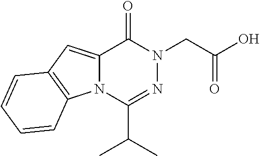 [SMILES: CC(C)c1nn(CC(=O)O)c(=O)c2cc3ccccc3n12] | 88 | 15D |
| B | 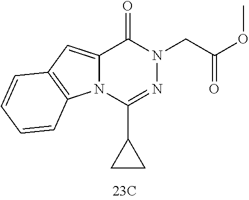 [SMILES: COC(=O)Cn1nc(C2CC2)n2c(cc3ccccc32)c1=O] | 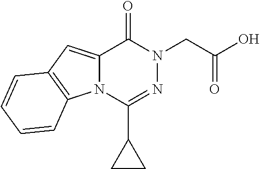 [SMILES: O=C(O)Cn1nc(C2CC2)n2c(cc3ccccc32)c1=O] | 99 | 16D |
| B | 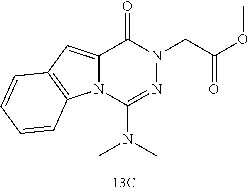 [SMILES: COC(=O)Cn1nc(N(C)C)n2c(cc3ccccc32)c1=O] | 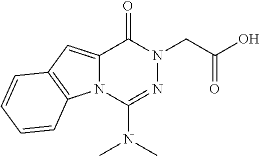 [SMILES: CN(C)c1nn(CC(=O)O)c(=O)c2cc3ccccc3n12] | 97 | 17D |
| C | 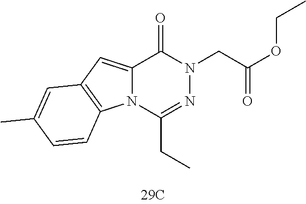 [SMILES: CCOC(=O)Cn1nc(CC)n2c(cc3cc(C)ccc32)c1=O] | 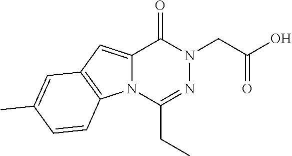 [SMILES: CCc1nn(CC(=O)O)c(=O)c2cc3cc(C)ccc3n12] | 52 | 18D |
| B^{a} | 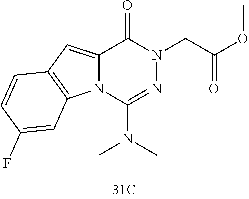 [SMILES: COC(=O)Cn1nc(N(C)C)n2c(cc3ccc(F)cc32)c1=O] | 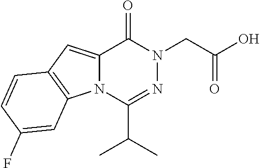 [SMILES: CC(C)c1nn(CC(=O)O)c(=O)c2cc3ccc(F)cc3n12] | 96 | 19D |
| C | 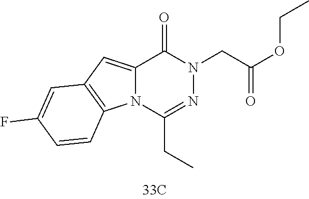 [SMILES: CCOC(=O)Cn1nc(CC)n2c(cc3cc(F)ccc32)c1=O] | 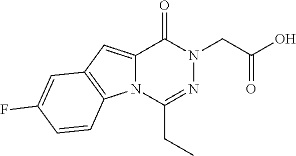 [SMILES: CCc1nn(CC(=O)O)c(=O)c2cc3cc(F)ccc3n12] | 84 | 20D |

^{a}Stirred at rt (not 90° C.) for 16 hours

### Synthesis of 2-[4-(1-ethoxyvinyl)-1-oxo-[1,2,4]triazino[4,5-a]indol-2-yl]-N-pyrimidin-4-1-acetamide 1E

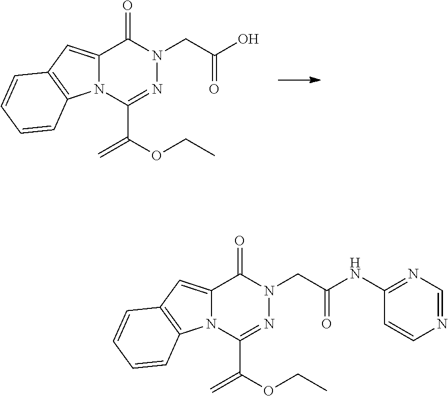 [SMILES: C=C(OCC)c1nn(CC(=O)O)c(=O)c2cc3ccccc3n12 ; C=C(OCC)c1nn(CC(=O)Nc2ccncn2)c(=O)c2cc3ccccc3n12]

1-propanephosphonic anhydride [68957-94-8] (0.61 mL, 0.96 mmol) was added to a stirred mixture of 2-[4-(1-ethoxyvinyl)-1-oxo-[1,2,4]triazino[4,5-a]indol-2-yl]acetic acid 4D (150 mg, 0.48 mmol), 4-aminopyrimidine [591-54-8] (82 mg, 0.86 mmol) and TEA [121-44-8] (0.13 mL, 0.96 mmol) in DCM (5 mL). The mixture was stirred at rt for 16 h. Then, a saturated aqueous solution of NaHCO₃ was added and the mixture was extracted with DCM. The organic layer was separated, dried (Na₂SO₄), filtered and the solvents evaporated in vacuo. The resulting solid was triturated with ACN, filtered and dried, filtered and dried under vacuo at 50° C. for 2 h to afford 2-[4-(1-ethoxyvinyl)-1-oxo-[1,2,4]triazino[4,5-a]indol-2-yl]-N-pyrimidin-4-yl-acetamide 1E (150 mg, 80%) as an off-white solid.

### Synthesis of 2-(4-acetyl-1-oxo-[1,2,4]triazino[4,5-a]indol-2-yl)-N-pyrimidin-4-yl-acetamide 2E

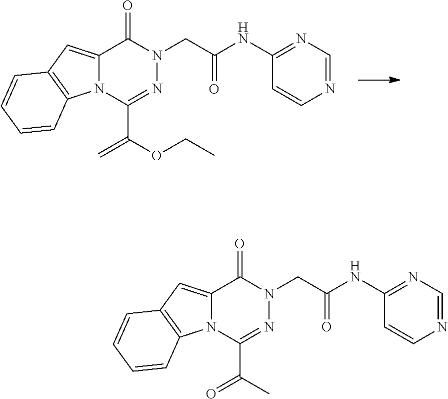 [SMILES: C=C(OCC)c1nn(CC(=O)Nc2ccncn2)c(=O)c2cc3ccccc3n12 ; CC(=O)c1nn(CC(=O)Nc2ccncn2)c(=O)c2cc3ccccc3n12]

A 2N aqueous solution of HCl [7647-01-0] (3 mL, 6 mmol) was added to a stirred solution of 2-[4-(1-ethoxyvinyl)-1-oxo-[1,2,4]triazino[4,5-a]indol-2-yl]-N-pyrimidin-4-yl-acetamide 1E (150 mg, 0.38 mmol) in THF (5 mL). The mixture was stirred at rt for 16 h. The mixture was extracted with EtOAc. The organic layer was separated, dried (MgSO4), filtered and the solvents evaporated in vacuo. The crude product was purified by flash column chromatography (silica; MeOH in DCM 0/100 to 4/96). The desired fractions were collected and concentrated in vacuo to yield 2-(4-acetyl-1-oxo-[1,2,4]triazino[4,5-a]indol-2-yl)-N-pyrimidin-4-yl-acetamide 2E (98 mg, 70%) as a white solid.

### Synthesis of 2-(1,4-dioxo-3H-[1,2,4]triazino[4,5-a]indol-2-yl)-N-tetrahydropyran-4-yl-acetamide 3E

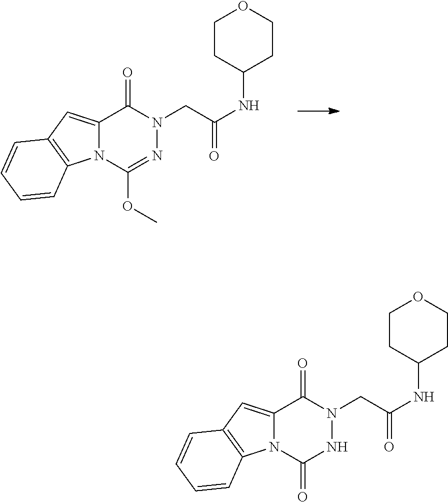 [SMILES: COc1nn(CC(=O)NC2CCOCC2)c(=O)c2cc3ccccc3n12 ; O=C(Cn1[nH]c(=O)n2c(cc3ccccc32)c1=O)NC1CCOCC1]

Chlorotrimethylsilane [75-77-4] (0.072 mL, 0.57 mmol) and NaI [7681-82-5] (85.13 mg, 0.57 mmol) were added to a stirred solution of 2-(4-methoxy-1-oxo-[1,2,4]triazino[4,5-a]indol-2-yl)-N-tetrahydropyran-4-yl-acetamide (Final compound 24) (100 mg, 0.26 mmol) in ACN (3.5 mL). The reaction was stirred at 80° C. for I h. The mixture was diluted with water and the formed precipitate was filtered, washed with water and dried under vacuo to afford methyl 2-(1,4-dioxo-3H-[1,2,4]triazino[4,5-a]indol-2-yl)-N-tetrahydropyran-4-yl-acetamide 3E (85 mg, 91%) as a white solid.

### Synthesis of [1-oxo-2-[2-oxo-2-(tetrahydropyran-4-ylamino)ethyl]-[1,2,4]triazino[4,5-a]indol-4-yl] trifluoromethanesulfonate 4E

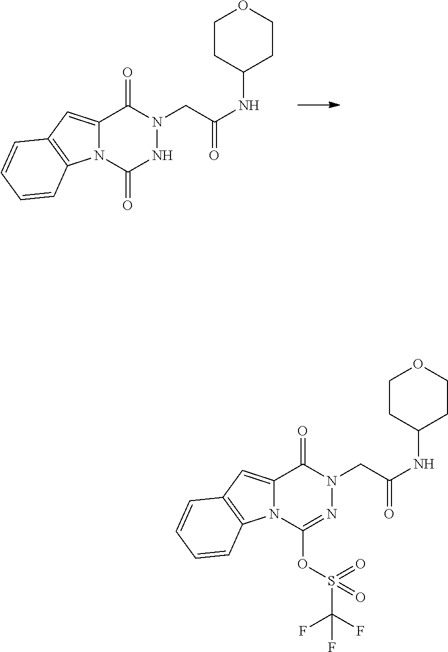 [SMILES: O=C(Cn1[nH]c(=O)n2c(cc3ccccc32)c1=O)NC1CCOCC1 ; O=C(Cn1nc(OS(=O)(=O)C(F)(F)F)n2c(cc3ccccc32)c1=O)NC1CCOCC1]

2-(1,4-Dioxo-3H-[1,2,4]triazino[4,5-a]indol-2-yl)-N-tetrahydropyran-4-yl-acetamide 3E (85 mg, 0.25 mmol) was dissolved in DCM (6.4 mL) and TEA [121-44-8] (35 μL, 0.25 mmol) in a vial that was cooled to 0° C. The vial was purged with N₂, and trifluoromethanesulfonic anhydride [358-23-6] (41 μL, 0.25 mmol) was added dropwise. The mixture was allowed to warm to room temperature and stirred for 5 min. Then, the mixture was concentrated in vacuo and purified by flash column chromatography (silica; MeOH in DCM 0/100 to 10/90). The desired fractions were collected and concentrated in vacuo to afford [1-oxo-2-[2-oxo-2-(tetrahydropyran-4-ylamino)ethyl]-[1,2,4]triazino[4,5-a]indol-4-yl] trifluoromethanesulfonate 4E (56 mg, 45%) as a yellow solid.

### Preparation of Final Compounds

### Synthesis of 2-(4-chloro-1-oxo-[1,2,4]triazino[4,5-a]indol-2-yl)-N-pyrimidin-4-yl-acetamide (Final Compound 1)

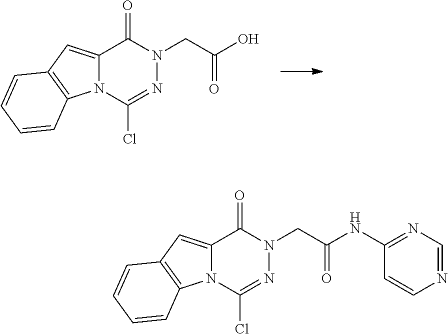 [SMILES: O=C(O)Cn1nc(Cl)n2c(cc3ccccc32)c1=O ; O=C(Cn1nc(Cl)n2c(cc3ccccc32)c1=O)Nc1ccncn1]

1-propanephosphonic anhydride [68957-94-8] (0.46 mL, 0.72 mmol) was added to a stirred mixture of 2-(4-chloro-1-oxo-[1,2,4]triazino[4,5-a]indol-2-yl)acetic acid 3D (100 mg, 0.36 mmol), 4-aminopyrimidine [591-54-8] (62 mg, 0.65 mmol) and TEA [121-44-8] (0.1 mL, 0.72 mmol) in DCM (3 mL). The mixture was stirred at rt for 16 h. Then, a saturated aqueous solution of NaHCO₃ was added and the product was extracted with DCM. The organic layer was separated, washed with water, dried (Na₂SO₄), filtered and the solvents evaporated in vacuo. The resulting solid was triturated with ACN, filtered and dried under vacuo at 50° C. for 2 h to yield 2-(4-chloro-1-oxo-[1,2,4]triazino[4,5-a]indol-2-yl)-N-pyrimidin-4-yl-acetamide (Final compound 1) (45 mg, 35%) as an off-white solid.

Structure analogs were synthesized using the same procedure:

| | | | Yield | Num- |
|---|---|---|---|---|
| Reagent | Intermediate | Final compound | % | ber |
| 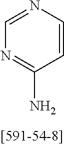 | 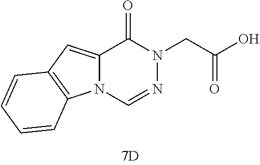 [SMILES: O=C(O)Cn1ncn2c(cc3ccccc32)c1=O] | 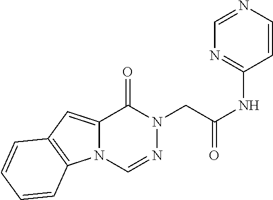 [SMILES: O=C(Cn1ncn2c(cc3ccccc32)c1=O)Nc1ccncn1] | 21 | 2 |
| 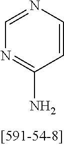 | 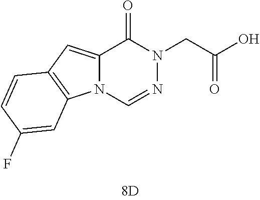 [SMILES: O=C(O)Cn1ncn2c(cc3ccc(F)cc32)c1=O] | 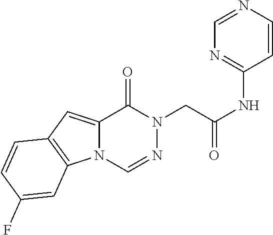 [SMILES: O=C(Cn1ncn2c(cc3ccc(F)cc32)c1=O)Nc1ccncn1] | 70 | 3 |
| 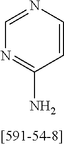 | 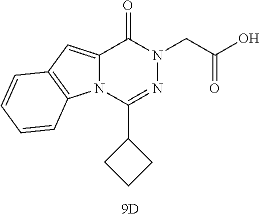 [SMILES: O=C(O)Cn1nc(C2CCC2)n2c(cc3ccccc32)c1=O] | 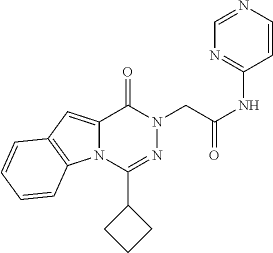 [SMILES: O=C(Cn1nc(C2CCC2)n2c(cc3ccccc32)c1=O)Nc1ccncn1] | 40 | 4 |
| 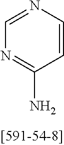 | 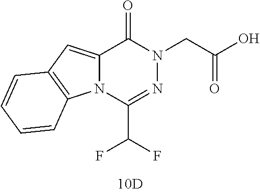 [SMILES: O=C(O)Cn1nc(C(F)F)n2c(cc3ccccc32)c1=O] | 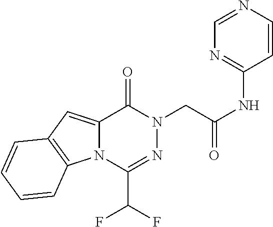 [SMILES: O=C(Cn1nc(C(F)F)n2c(cc3ccccc32)c1=O)Nc1ccncn1] | 7 | 5 |
| 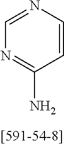 | 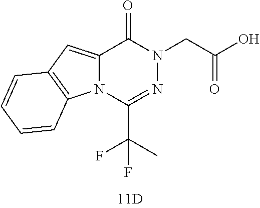 [SMILES: CC(F)(F)c1nn(CC(=O)O)c(=O)c2cc3ccccc3n12] | 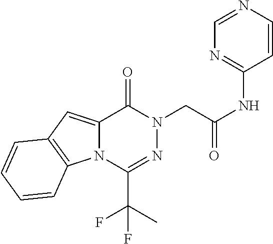 [SMILES: CC(F)(F)c1nn(CC(=O)Nc2ccncn2)c(=O)c2cc3ccccc3n12] | 8 | 6 |
| 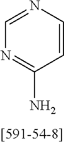 | 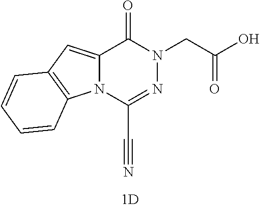 [SMILES: N#Cc1nn(CC(=O)O)c(=O)c2cc3ccccc3n12] | 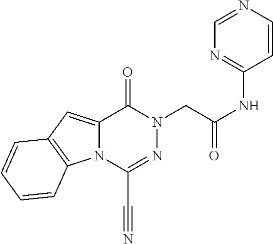 [SMILES: N#Cc1nn(CC(=O)Nc2ccncn2)c(=O)c2cc3ccccc3n12] | 70 | 7 |
| 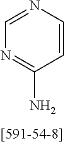 | 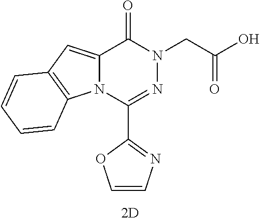 [SMILES: O=C(O)Cn1nc(-c2ncco2)n2c(cc3ccccc32)c1=O] | 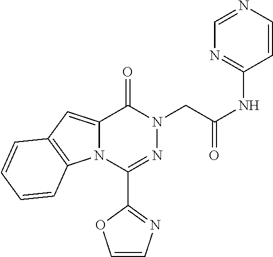 [SMILES: O=C(Cn1nc(-c2ncco2)n2c(cc3ccccc32)c1=O)Nc1ccncn1] | 32 | 8 |
| 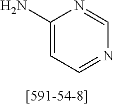 | 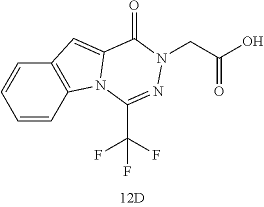 [SMILES: O=C(O)Cn1nc(C(F)(F)F)n2c(cc3ccccc32)c1=O] | 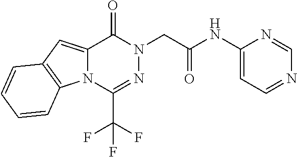 [SMILES: O=C(Cn1nc(C(F)(F)F)n2c(cc3ccccc32)c1=O)Nc1ccncn1] | 30 | 9 |
| 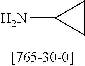 | 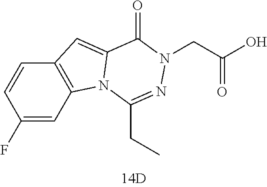 [SMILES: CCc1nn(CC(=O)O)c(=O)c2cc3ccc(F)cc3n12] | 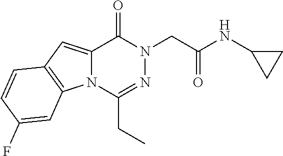 [SMILES: CCc1nn(CC(=O)NC2CC2)c(=O)c2cc3ccc(F)cc3n12] | 50 | 10 |
| 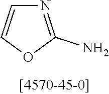 | 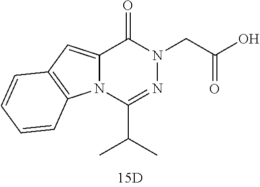 [SMILES: CC(C)c1nn(CC(=O)O)c(=O)c2cc3ccccc3n12] | 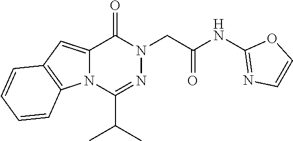 [SMILES: CC(C)c1nn(CC(=O)Nc2ncco2)c(=O)c2cc3ccccc3n12] | 25 | 11 |
| 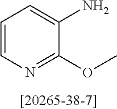 | 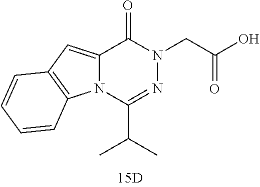 [SMILES: CC(C)c1nn(CC(=O)O)c(=O)c2cc3ccccc3n12] | 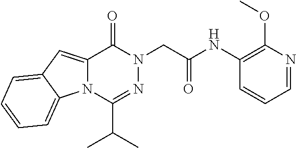 [SMILES: COc1ncccc1NC(=O)Cn1nc(C(C)C)n2c(cc3ccccc32)c1=O] | 57 | 12 |
| 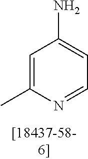 | 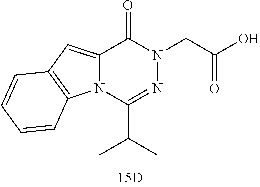 [SMILES: CC(C)c1nn(CC(=O)O)c(=O)c2cc3ccccc3n12] | 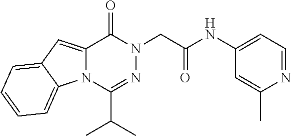 [SMILES: Cc1cc(NC(=O)Cn2nc(C(C)C)n3c(cc4ccccc43)c2=O)ccn1] | 33 | 13 |
| 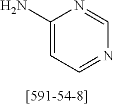 | 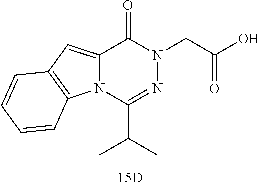 [SMILES: CC(C)c1nn(CC(=O)O)c(=O)c2cc3ccccc3n12] | 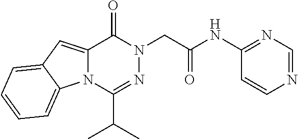 [SMILES: CC(C)c1nn(CC(=O)Nc2ccncn2)c(=O)c2cc3ccccc3n12] | 52 | 14 |
| 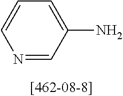 | 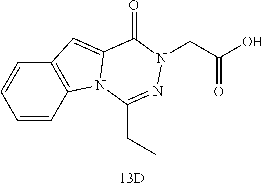 [SMILES: CCc1nn(CC(=O)O)c(=O)c2cc3ccccc3n12] | 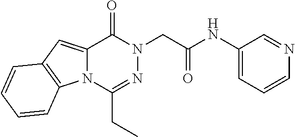 [SMILES: CCc1nn(CC(=O)Nc2cccnc2)c(=O)c2cc3ccccc3n12] | 55 | 15 |
| 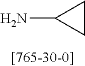 | 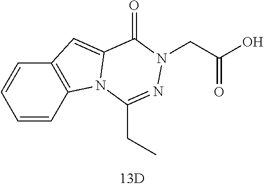 [SMILES: CCc1nn(CC(=O)O)c(=O)c2cc3ccccc3n12] | 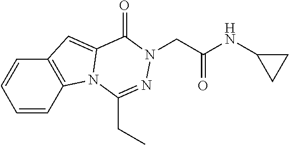 [SMILES: CCc1nn(CC(=O)NC2CC2)c(=O)c2cc3ccccc3n12] | 71 | 16 |
| 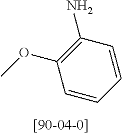 | 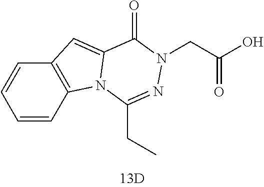 [SMILES: CCc1nn(CC(=O)O)c(=O)c2cc3ccccc3n12] | 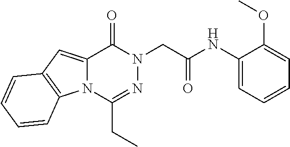 [SMILES: CCc1nn(CC(=O)Nc2ccccc2OC)c(=O)c2cc3ccccc3n12] | 85 | 17 |
| 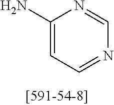 | 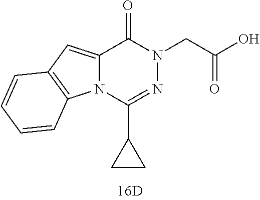 [SMILES: O=C(O)Cn1nc(C2CC2)n2c(cc3ccccc32)c1=O] | 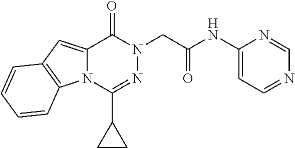 [SMILES: O=C(Cn1nc(C2CC2)n2c(cc3ccccc32)c1=O)Nc1ccncn1] | 37 | 18 |
| 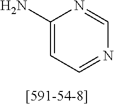 | 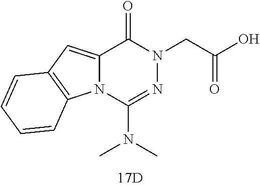 [SMILES: CN(C)c1nn(CC(=O)O)c(=O)c2cc3ccccc3n12] | 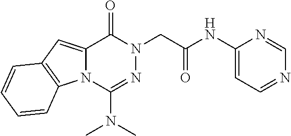 [SMILES: CN(C)c1nn(CC(=O)Nc2ccncn2)c(=O)c2cc3ccccc3n12] | 10 | 19 |
| 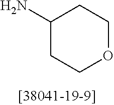 | 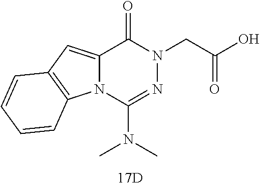 [SMILES: CN(C)c1nn(CC(=O)O)c(=O)c2cc3ccccc3n12] | 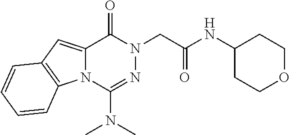 [SMILES: CN(C)c1nn(CC(=O)NC2CCOCC2)c(=O)c2cc3ccccc3n12] | 98 | 20 |
| 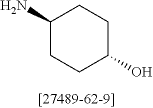 | 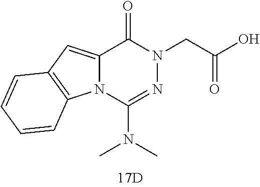 [SMILES: CN(C)c1nn(CC(=O)O)c(=O)c2cc3ccccc3n12] | 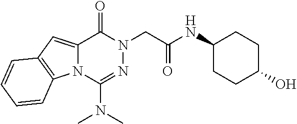 [SMILES: CN(C)c1nn(CC(=O)N[C@H]2CC[C@H](O)CC2)c(=O)c2cc3ccccc3n12] | 97 | 21 |
| 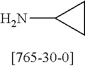 | 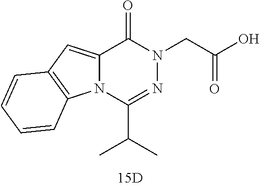 [SMILES: CC(C)c1nn(CC(=O)O)c(=O)c2cc3ccccc3n12] | 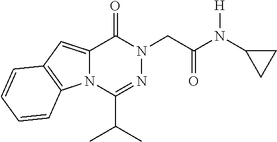 [SMILES: CC(C)c1nn(CC(=O)NC2CC2)c(=O)c2cc3ccccc3n12] | 57 | 29 |
| 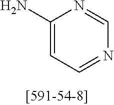 | 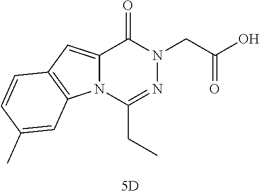 [SMILES: CCc1nn(CC(=O)O)c(=O)c2cc3ccc(C)cc3n12] | 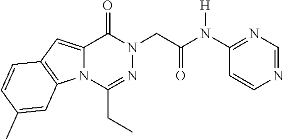 [SMILES: CCc1nn(CC(=O)Nc2ccncn2)c(=O)c2cc3ccc(C)cc3n12] | 56 | 30 |
| 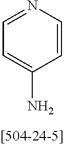 | 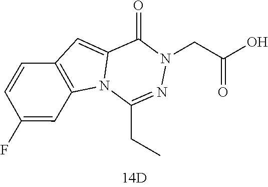 [SMILES: CCc1nn(CC(=O)O)c(=O)c2cc3ccc(F)cc3n12] | 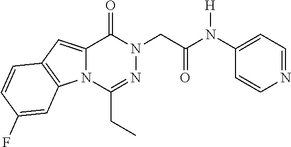 [SMILES: CCc1nn(CC(=O)Nc2ccncc2)c(=O)c2cc3ccc(F)cc3n12] | 92 | 31 |
| 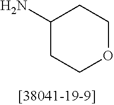 | 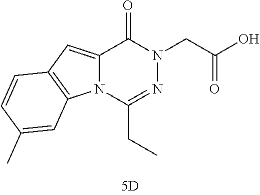 [SMILES: CCc1nn(CC(=O)O)c(=O)c2cc3ccc(C)cc3n12] | 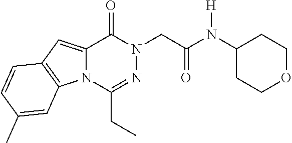 [SMILES: CCc1nn(CC(=O)NC2CCOCC2)c(=O)c2cc3ccc(C)cc3n12] | 42 | 32 |
| 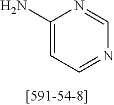 | 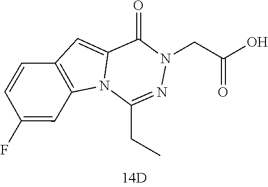 [SMILES: CCc1nn(CC(=O)O)c(=O)c2cc3ccc(F)cc3n12] | 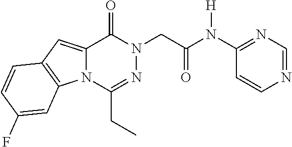 [SMILES: CCc1nn(CC(=O)Nc2ccncn2)c(=O)c2cc3ccc(F)cc3n12] | 93 | 33 |
| 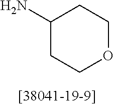 | 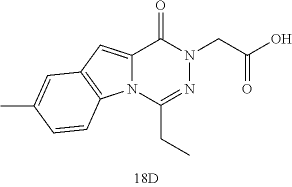 [SMILES: CCc1nn(CC(=O)O)c(=O)c2cc3cc(C)ccc3n12] | 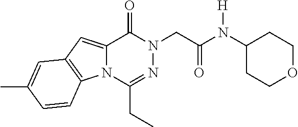 [SMILES: CCc1nn(CC(=O)NC2CCOCC2)c(=O)c2cc3cc(C)ccc3n12] | 81 | 34 |
| 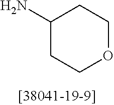 | 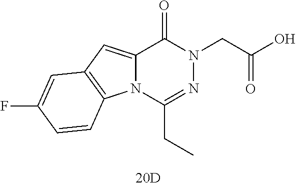 [SMILES: CCc1nn(CC(=O)O)c(=O)c2cc3cc(F)ccc3n12] | 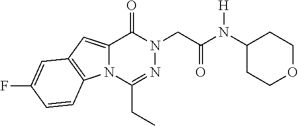 [SMILES: CCc1nn(CC(=O)NC2CCOCC2)c(=O)c2cc3cc(F)ccc3n12] | 85 | 35 |
| 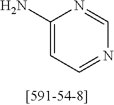 | 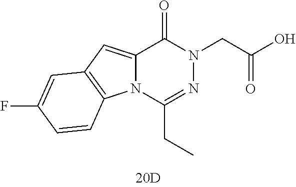 [SMILES: CCc1nn(CC(=O)O)c(=O)c2cc3cc(F)ccc3n12] | 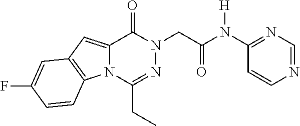 [SMILES: CCc1nn(CC(=O)Nc2ccncn2)c(=O)c2cc3cc(F)ccc3n12] | 43 | 36 |
| 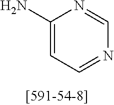 | 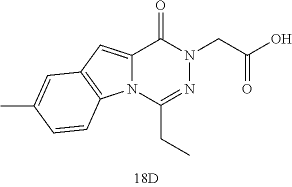 [SMILES: CCc1nn(CC(=O)O)c(=O)c2cc3cc(C)ccc3n12] | 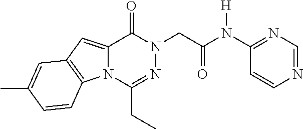 [SMILES: CCc1nn(CC(=O)Nc2ccncn2)c(=O)c2cc3cc(C)ccc3n12] | 38 | 37 |
| 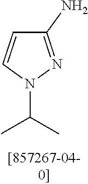 | 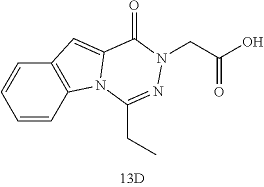 [SMILES: CCc1nn(CC(=O)O)c(=O)c2cc3ccccc3n12] | 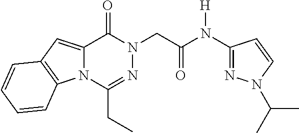 [SMILES: CCc1nn(CC(=O)Nc2ccn(C(C)C)n2)c(=O)c2cc3ccccc3n12] | 19^{a} | 38 |

^{a}Reaction in DMF

### Synthesis of 2-{4-ethyl-7-fluoro-1-oxo-[1,2,4]triazino[4,5-a]indol-2(1H)-yl}-N-((1s,3s)-3-hydroxy-3-methylcyclobutyl)acetamide (Final Compound 39)

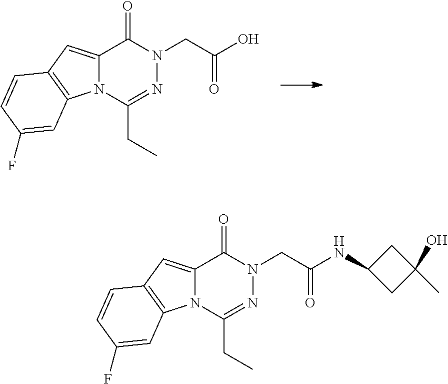 [SMILES: CCc1nn(CC(=O)O)c(=O)c2cc3ccc(F)cc3n12 ; CCc1nn(CC(=O)N[C@H]2C[C@@](C)(O)C2)c(=O)c2cc3ccc(F)cc3n12]

HATU (N-[(dimethylamino)-1H-1,2,3-triazolo-[4,5-b]pyridin-1-ylmethylene]-N-methylmethanaminium hexafluorophosphate N-oxide) [148893-10-1] (197 mg, 0.52 mmol), cis-3-hydroxy-3-methylcyclobutylamine hydrochloride [1363381-58-1] (57 mg, 0.41 mmol) and diisopropyl ethylamine (DIPEA) [7087-68-5] (0.3 mL, 1.72 mmol) were added sequentially to a stirred mixture of 2-(4-ethyl-7-fluoro-1-oxo-[1,2,4]triazino[4,5-a]indol-2(1H)-yl)acetic acid 14D (100 mg, 0.34 mmol) in DMF (2 mL) at rt. The mixture was stirred at rt for 18 h. Then, DI water was added and the formed precipitate was filtered off, washed with DI water and diethyl ether to yield 2-{4-ethyl-7-fluoro-1-oxo-[1,2,4]triazino[4,5-a]indol-2(1H)-yl}-N-((1s,3s)-3-hydroxy-3-methylcyclobutyl)acetamide (Final compound 39) (50 mg, 39%) as a solid.

If required, the obtained solid was further purified by reverse-phase preparative HPLC.

Structure analogs were synthesized using the same procedure:

| | | | Yield | Num- |
|---|---|---|---|---|
| Reagent | Intermediate | Final compound | % | ber |
| 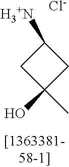 | 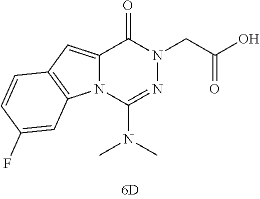 [SMILES: CN(C)c1nn(CC(=O)O)c(=O)c2cc3ccc(F)cc3n12] | 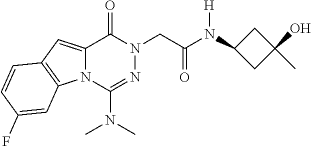 [SMILES: CN(C)c1nn(CC(=O)N[C@H]2C[C@@](C)(O)C2)c(=O)c2cc3ccc(F)cc3n12] | 29 | 40 |
| 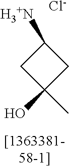 | 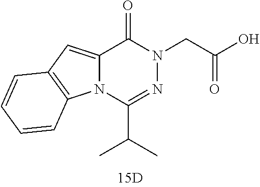 [SMILES: CC(C)c1nn(CC(=O)O)c(=O)c2cc3ccccc3n12] | 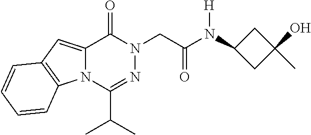 [SMILES: CC(C)c1nn(CC(=O)N[C@H]2C[C@@](C)(O)C2)c(=O)c2cc3ccccc3n12] | 81 | 41 |
| 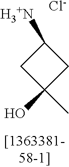 | 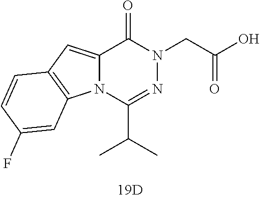 [SMILES: CC(C)c1nn(CC(=O)O)c(=O)c2cc3ccc(F)cc3n12] | 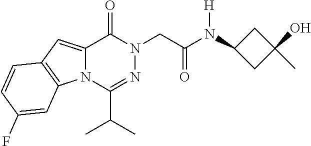 [SMILES: CC(C)c1nn(CC(=O)N[C@H]2C[C@@](C)(O)C2)c(=O)c2cc3ccc(F)cc3n12] | 13 | 42 |
| 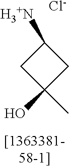 | 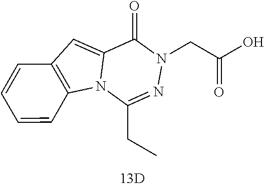 [SMILES: CCc1nn(CC(=O)O)c(=O)c2cc3ccccc3n12] | 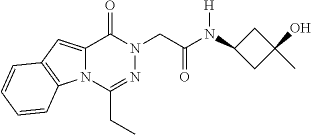 [SMILES: CCc1nn(CC(=O)N[C@H]2C[C@@](C)(O)C2)c(=O)c2cc3ccccc3n12] | 93 | 43 |
| 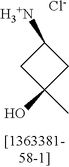 | 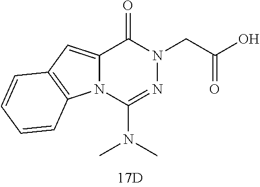 [SMILES: CN(C)c1nn(CC(=O)O)c(=O)c2cc3ccccc3n12] | 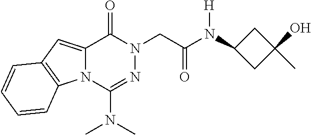 [SMILES: CN(C)c1nn(CC(=O)N[C@H]2C[C@@](C)(O)C2)c(=O)c2cc3ccccc3n12] | 93 | 44 |
| 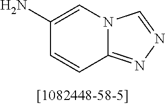 | 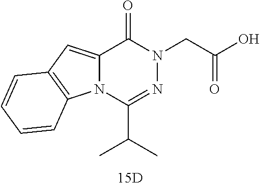 [SMILES: CC(C)c1nn(CC(=O)O)c(=O)c2cc3ccccc3n12] | 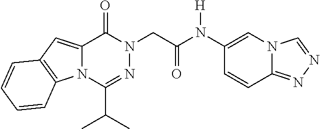 [SMILES: CC(C)c1nn(CC(=O)Nc2ccc3nncn3c2)c(=O)c2cc3ccccc3n12] | 85 | 45 |
| 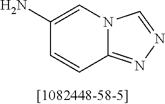 | 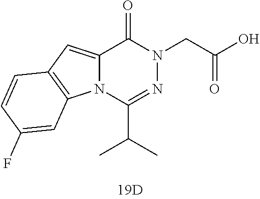 [SMILES: CC(C)c1nn(CC(=O)O)c(=O)c2cc3ccc(F)cc3n12] | 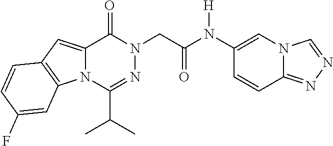 [SMILES: CC(C)c1nn(CC(=O)Nc2ccc3nncn3c2)c(=O)c2cc3ccc(F)cc3n12] | 37 | 46 |
| 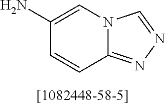 | 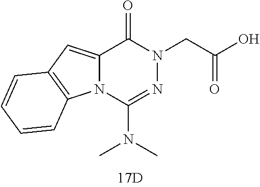 [SMILES: CN(C)c1nn(CC(=O)O)c(=O)c2cc3ccccc3n12] | 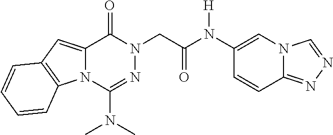 [SMILES: CN(C)c1nn(CC(=O)Nc2ccc3nncn3c2)c(=O)c2cc3ccccc3n12] | 39 | 47 |
| 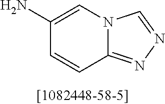 | 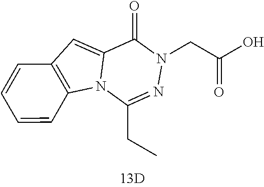 [SMILES: CCc1nn(CC(=O)O)c(=O)c2cc3ccccc3n12] | 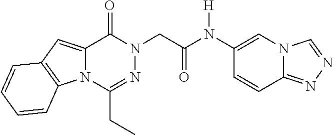 [SMILES: CCc1nn(CC(=O)Nc2ccc3nncn3c2)c(=O)c2cc3ccccc3n12] | 63 | 48 |

### Synthesis of N-([1,2,4]triazolo[4,3-b]pyridazin-6-yl)-2-(4-isopropyl-1-oxo-[1,2,4]triazino[4,5-a]indol-2(1H)-yl)acetamide (Final Compound 49)

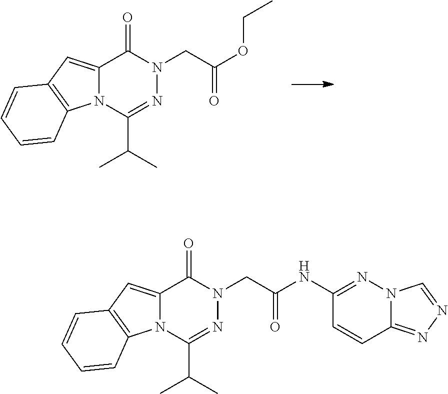 [SMILES: CCOC(=O)Cn1nc(C(C)C)n2c(cc3ccccc32)c1=O ; CC(C)c1nn(CC(=O)Nc2ccc3nncn3n2)c(=O)c2cc3ccccc3n12]

[1,2,4]Triazolo[4,3-b]pyridazin-6-amine [19195-46-1] (63.6 mg, 0.45 mmol) was placed in a dry 8-mL MW vial equipped with a magnetic stir bar and the setup placed under nitrogen (3 vacuum/nitrogen cycles). Anhydrous DMF (1.24 mL) was added and the solution cooled to 0° C. After 2 minutes at 0° C., a solution of LiHMDS (0.77 mL, 1 M in THF, 0.77 mmol) was added dropwise and the resulting solution stirred at 0° C. for further 2 minutes, whereupon a fine suspension had formed. Then, a solution of ethyl 2-(4-isopropyl-1-oxo-[1,2,4]triazino[4,5-a]indol-2(1H)-yl)acetate 32C (100 mg, 0.32 mmol) in anhydrous THF (1 mL) was added dropwise at 0° C. over 2 minutes. The resulting mixture was allowed to warm to r.t. and stirred at r.t. for 2 hours.

The crude mixture was quenched by addition of DI water (3 mL) then 1M aqueous HCl (0.7 mL overall). The resulting pale brown mixture was extracted with DCM (10×5 mL). The combined organic extracts were concentrated in vacuo. The obtained solid was redissolved in hot acetonitrile (ca. 35 mL), the hot solution was filtered off to remove insolubles and the resulting solution was allowed to cool to r.t. with vigorous stirring and stirred at r.t. for overall 16 hours. The resulting suspension was filtered off to give the title amide 49 (76 mg, 59%) as a pale tan solid.

### Synthesis of 2-[4-(1-hydroxyethyl)-1-oxo-[1,2,4]triazino[4,5-a]indol-2-yl]-N-pyrimidin-4-yl-acetamide (Final Compound 22)

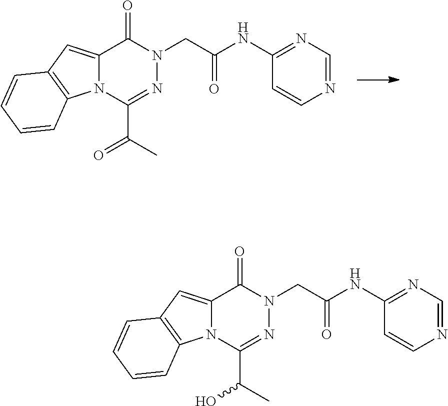 [SMILES: CC(=O)c1nn(CC(=O)Nc2ccncn2)c(=O)c2cc3ccccc3n12 ; CC(O)c1nn(CC(=O)Nc2ccncn2)c(=O)c2cc3ccccc3n12]

Sodium borohydride [16940-66-2] (11 mg, 0.3 mmol) was added to a stirred solution 2-(4-acetyl-1-oxo-[1,2,4]triazino[4,5-a]indol-2-yl)-N-pyrimidin-4-yl-acetamide 2E (90 mg, 0.25 mmol) in MeOH (5.4 mL) at 0° C. The mixture was stirred at rt for 1 h. Then, the mixture was diluted with a saturated aqueous solution of NH₄Cl and extracted with EtOAc. The organic layer was separated, dried (MgSO₄), filtered and the solvents evaporated in vacuo. The crude product was purified by flash column chromatography (silica; MeOH in DCM 0/100 to 2/98). The desired fractions were collected and concentrated in vacuo to yield 2-[4-(1-hydroxyethyl)-1-oxo-[1,2,4]triazino[4,5-a]indol-2-yl]-N-pyrimidin-4-yl-acetamide (Final compound 22) (52 mg, 57%) as a white solid.

### Synthesis of N-cyclopropyl-2-(4-methyl-1-oxo-[1,2,4]triazino[4,5-a]indol-2-yl)acetamide (Final compound 23)

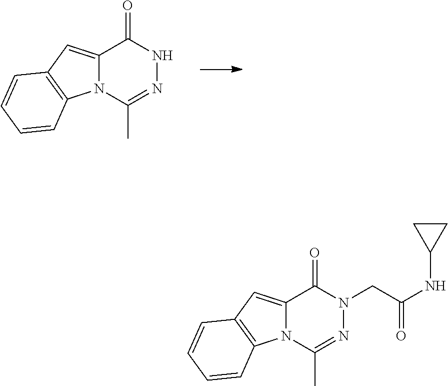 [SMILES: Cc1n[nH]c(=O)c2cc3ccccc3n12 ; Cc1nn(CC(=O)NC2CC2)c(=O)c2cc3ccccc3n12]

A mixture of 4-methyl-2H-[1,2,4]triazino[4,5-a]indol-1-one [37574-74-6] (100 mg, 0.50 mmol), 2-chloro-N-cyclopropyl-acetamide [19047-31-5] (80 mg, 0.60 mmol), 18-crown-6 [17455-13-9] (7 mg, 0.025 mmol), KI [7681-11-0] (10 mg, 0.06 mmol) and K₂CO₃ [584-08-7] (83 mg, 0.60 mmol) in ACN (8 mL) was stirred at 116° C. for 16 h. The mixture was diluted with water and extracted with DCM, the organic layer was separated, dried (Na₂SO₄), filtered and the solvents evaporated in vacuo to afford N-cyclopropyl-2-(4-methyl-1-oxo-[1,2,4]triazino[4,5-a]indol-2-yl)acetamide (Final compound 23) (102 mg, 69%) as cream.

Structure analog was synthesized using the same procedure:

| Reagent | Intermediate | Final compound | yield | Number |
|---|---|---|---|---|
| 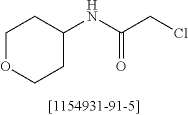 [SMILES: O=C(CCl)NC1CCOCC1] | 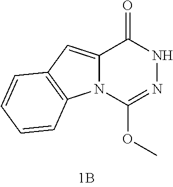 [SMILES: COc1n[nH]c(=O)c2cc3ccccc3n12] | 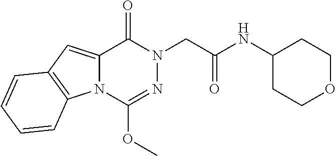 [SMILES: COc1nn(CC(=O)NC2CCOCC2)c(=O)c2cc3ccccc3n12] | 53 | 24 |
| 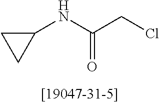 | 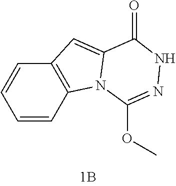 [SMILES: COc1n[nH]c(=O)c2cc3ccccc3n12] | 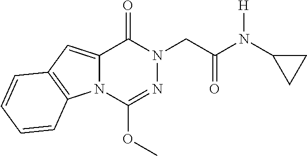 [SMILES: COc1nn(CC(=O)NC2CC2)c(=O)c2cc3ccccc3n12] | 8 | 50 |

Structure analog were synthesized using the same procedure:

| | | | Yield | |
|---|---|---|---|---|
| Reagent | Intermediate | Final compound | % | No. |
| 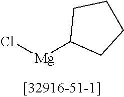 | 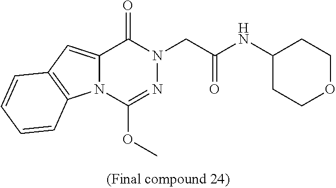 [SMILES: COc1nn(CC(=O)NC2CCOCC2)c(=O)c2cc3ccccc3n12] | 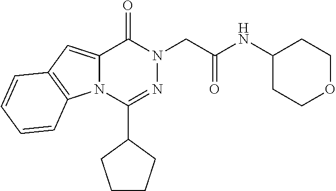 [SMILES: O=C(Cn1nc(C2CCCC2)n2c(cc3ccccc32)c1=O)NC1CCOCC1] | 5 | 25 |
| 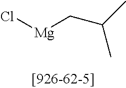 | 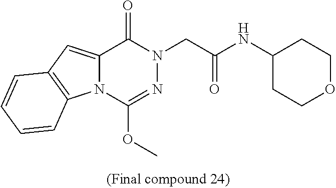 [SMILES: COc1nn(CC(=O)NC2CCOCC2)c(=O)c2cc3ccccc3n12] | 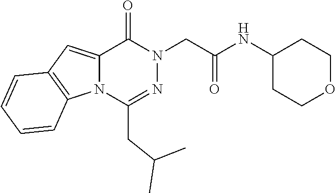 [SMILES: CC(C)Cc1nn(CC(=O)NC2CCOCC2)c(=O)c2cc3ccccc3n12] | 15 | 26 |
| 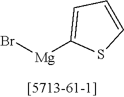 | 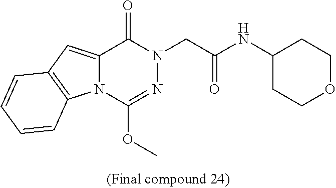 [SMILES: COc1nn(CC(=O)NC2CCOCC2)c(=O)c2cc3ccccc3n12] | 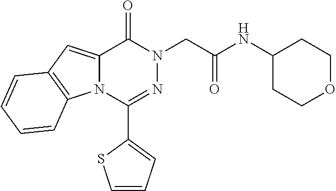 [SMILES: O=C(Cn1nc(-c2cccs2)n2c(cc3ccccc32)c1=O)NC1CCOCC1] | 7 | 27 |
| 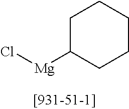 | 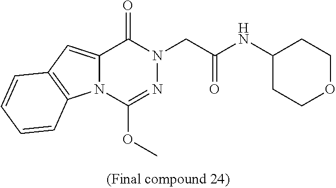 [SMILES: COc1nn(CC(=O)NC2CCOCC2)c(=O)c2cc3ccccc3n12] | 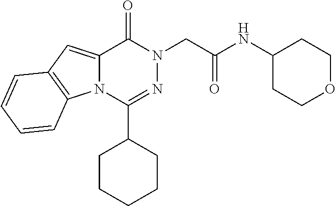 [SMILES: O=C(Cn1nc(C2CCCCC2)n2c(cc3ccccc32)c1=O)NC1CCOCC1] | 19 | 28 |

Characterising Data—LC-MS and Melting Point

LCMS: [M+H]⁺ means the protonated mass of the free base of the compound, Rₜ means retention time (in minutes), method refers to the method used for LCMS.

| | M.p | | | | LCMS |
|---|---|---|---|---|---|
| Final Cpd | (° C.) | [M + H]⁺ | [M − H]⁻ | Rₜ | Method |
| 1 | n.t. | 355 | — | 1.08 | A |
| 2 | n.t. | 321 | — | 0.82 | B |
| 3 | n.t. | 339 | — | 0.87 | B |
| 4 | n.t. | 375 | — | 2.01 | D |
| 5 | n.t. | 371 | — | 1.05 | E |
| 6 | n.t. | 385 | — | 1.23 | B |
| 7 | n.t. | 346 | — | 1.70 | D |
| 9 | n.t. | 389 | 387 | 1.85 | F |
| 10 | 277.81 | 329 | 327 | 1.67 | F |
| 11 | n.t. | 352 | 350 | 1.82 | G |
| 12 | n.t. | 392 | 390 | 2.55 | H |
| 13 | n.t. | 376 | 374 | 2.12 | H |
| 14 | n.t. | 363 | 361 | 1.94 | G |
| 15 | 232.81 | 348 | 346 | 1.61 | F |
| 16 | 255.58 | 311 | 309 | 1.61 | F |
| 17 | 216.79 | 377 | — | 1.96 | F |
| 18 | n.t. | 361 | — | 1.70 | D |
| 19 | n.t. | 364 | — | 1.74 | D |
| 20 | n.t. | — | 368 | 1.07 | I |
| 21 | n.t. | — | 382 | 1.53 | I |
| 22 | n.t. | 365 | 363 | 0.83 | B |
| 23 | n.t. | 297 | — | 0.92 | A |
| 24 | n.t. | 357 | — | 1.44 | D |
| 25 | n.t. | 395 | 393 | 1.60 | K |
| 26 | n.t. | 383 | 381 | 0.81 | J |
| 27 | n.t. | 409 | 407 | 0.81 | J |
| 28 | n.t. | 409 | 407 | 0.95 | J |
| 29 | n.t. | 325 | — | 1.90 | G |
| 30 | 248.91 | 363 | 361 | 1.76 | F |
| 31 | n.t. | 366 | 364 | 1.64 | F |
| 32 | 264.11 | 369 | 367 | 1.70 | F |
| 33 | n.t. | 367 | — | 1.14 | A |
| 34 | 265.70 11& | 369 | 367 | 1.75 | F |
| | 274.18 | | | | |
| 35 | 285.66 | 373 | 371 | 1.65 | F |
| 36 | n.t. | 367 | 365 | 1.69 | F |
| 37 | n.t. | 363 | 361 | 1.80 | F |
| 38 | 243.0 | 379 | — | 3.15 | N |
| 39 | n.t. | 373 | — | 1.45 | D |
| 40 | n.t. | — | 386 | 1.55 | I |
| 41 | n.t. | 369 | — | 1.61 | D |
| 42 | n.t. | 387 | 385 | 1.51 | O |
| 43 | n.t. | 355 | 353 | 1.33/1.36 | D/I |
| 44 | n.t. | — | 368 | 1.60 | I |
| 45 | n.t. | 402 | 400 | 1.05 | B |
| 46 | n.t. | 420 | 418 | 1.84/1.84 | D/I |
| 47 | n.t. | 403 | 401 | 1.61/1.62 | D/I |
| 48 | n.t. | 388 | 386 | 1.54/1.54 | D/I |
| 49 | n.t. | 403 | 401 | 1.61 | L |
| 50 | n.t. | 313 | — | 1.03 | A |

Characterising Data—Compound+NMR

This is depicted in the following table:

| | |
|---|---|
| Final Cpd 1 | ¹H NMR (500 MHz, DMSO-d₆) δ ppm 4.93 (s, 2 H) 7.50-7.54 (m, 1 |
| | H) 7.61 (ddd, J = 8.6, 7.2, 1.3 Hz, 1 H) 7.66 (d, J = 0.6 Hz, 1 H) 7.97 (br |
| | d, J = 8.1 Hz, 1 H) 8.00 (dd, J = 5.8, 1.2 Hz, 1 H) 8.52 (dq, J = 8.7, 0.8 Hz, |
| | 1 H) 8.68 (d, J = 5.8 Hz, 1 H) 8.92 (d, J = 1.1 Hz, 1 H) 11.28 (br s, 1 H) |
| Final Cpd 2 | ¹H NMR (500 MHz, DMSO-d₆) δ ppm 4.94 (s, 2 H) 7.42-7.48 (m, 2 |
| | H) 7.57 (ddd, J = 8.4, 7.2, 1.1 Hz, 1 H) 7.91 (d, J = 8.1 Hz, 1 H) 7.99 (dd, |
| | J = 5.8, 0.9 Hz, 1 H) 8.24 (dd, J = 8.5, 0.7 Hz, 1 H) 8.65 (d, J = 5.8 Hz, 1 |
| | H) 8.91 (d, J = 0.6 Hz, 1 H) 9.20 (s, 1 H) 11.27 (br s, 1 H) |
| Final Cpd 3 | ¹H NMR (500 MHz, DMSO-d₆) δ ppm 4.92 (s, 2 H) 7.35 (td, J = 9.2, |
| | 2.4 Hz, 1 H) 7.47 (s, 1 H) 7.95 (dd, J = 8.9, 5.3 Hz, 1 H) 7.98 (dd, J = 5.8, |
| | 1.2 Hz, 1 H) 8.17 (dd, J = 9.6, 2.1 Hz, 1 H) 8.65 (d, J = 5.8 Hz, 1 H) 8.90 |
| | (d, J = 0.9 Hz, 1 H) 9.12 (s, 1 H) 11.22 (br s, 1 H) |
| Final Cpd 4 | ¹H NMR (500 MHz, DMSO-d₆) δ ppm 1.93 (ddq, J = 14.5, 9.6, 4.9, 4.9, |
| | 4.9 Hz, 1 H) 2.02-2.15 (m, 1 H) 2.34-2.45 (m, 2 H) 2.45-2.57 (m, 2 |
| | H) 4.32 (quin, J = 7.7 Hz, 1 H) 4.96 (s, 2 H) 7.40-7.47 (m, 1 H) 7.52 (s, |
| | 1 H) 7.55 (ddd, J = 8.5, 7.2, 1.2 Hz, 1 H) 7.92 (d, J = 7.9 Hz, 1 H) 8.01 |
| | (dd, J = 5.8, 1.1 Hz, I H) 8.05 (d, J = 8.5 Hz, 1 H) 8.67 (d, J = 5.8 Hz, 1 H) |
| | 8.92 (d, J = 0.9 Hz, 1 H) 11.29 (s, 1 H) |
| Final Cpd 5 | ¹H NMR (400 MHz, CDCl₃) δ ppm 4.96 (s, 2 H) 6.74 (t, J = 52.7 Hz, 1 |
| | H) 7.45-7.51 (m, 1 H) 7.58 (ddd, J = 8.7, 7.2, 1.3 Hz, 1 H) 7.72 (d, |
| | J = 0.9 Hz, 1 H) 7.88 (dt, J = 8.1, 0.9 Hz, 1 H) 8.13 (dd, J = 5.8, 0.9 Hz, 1 |
| | H) 8.14-8.19 (m, 1 H) 8.60-8.66 (m, 1 H) 8.65 (d, J = 5.8 Hz, 1 H) |
| | 8.88 (d, J = 1.2 Hz, 1 H) |
| Final Cpd 6 | ¹H NMR (500 MHz, CDCl₃) δ ppm 2.22 (t, J = 19.3 Hz, 3 H) 4.96 (s, 2 |
| | H) 7.44-7.50 (m, 1 H) 7.53-7.59 (m, 1 H) 7.74 (d, J = 0.6 Hz, 1 H) |
| | 7.86 (d, J = 7.9 Hz, 1 H) 8.14 (d, J = 5.6 Hz, 1 H) 8.24-8.30 (m, 1 H) |
| | 8.62-8.66 (m, 1 H) 8.66 (d, J = 5.8 Hz, 1 H) 8.89 (d, J = 1.2 Hz, 1 H) |
| Final Cpd 7 | ¹H NMR (400 MHz, DMSO-d₆) δ ppm 5.05 (s, 2 H) 7.55 (t, J = 7.5 Hz, |
| | 1 H) 7.67-7.73 (m, 2 H) 7.98-8.02 (m, 2 H) 8.34 (br dd, J = 8.7, 0.7 |
| | Hz, 1 H) 8.69 (d, J = 5.8 Hz, 1 H) 8.93 (d, J = 0.9 Hz, 1 H) 11.32 (s, 1 H) |
| Final Cpd 8 | ¹H NMR (400 MHz, METHANOL-d₄) δ ppm 5.06 (s, 2 H) 6.74 (dq, |
| | J = 8.4, 0.8 Hz, 1 H) 7.35-7.40 (m, 1 H) 7.40-7.46 (m, 1 H) 7.63 (d, |
| | J = 0.9 Hz, 1 H) 7.67 (d, J = 0.7 Hz, 1 H) 7.90-7.94 (m, 1 H) 8.15 (dd, |
| | J = 6.0, 0.9 Hz, 1 H) 8.33 (d, J = 0.9 Hz, 1 H) 8.60 (d, J = 6.0 Hz, 1 H) |
| | 8.85 (d, J = 0.9 Hz, 1 H), 1H exchanged |
| Final Cpd 9 | ¹H NMR (400 MHz, CDCl₃) δ ppm 4.99 (s, 2 H) 7.47-7.53 (m, 1 H) |
| | 7.57-7.63 (m, 1 H) 7.75 (d, J = 0.8 Hz, 1 H) 7.87-7.92 (m, 1 H) 7.97-8.02 |
| | (m, 1 H) 8.12 (d, J = 5.7 Hz, 1 H) 8.39 (br s, 1 H) 8.65 (d, J = 5.7 |
| | Hz, 1 H) 8.89 (d, J = 1.2 Hz, 1 H) |
| Final Cpd 10 | ¹H NMR (400 MHz, DMSO-d₆) δ ppm 0.35-0.48 (m, 2 H) 0.55-0.70 |
| | (m, 2 H) 1.29 (t, J = 7.2 Hz, 3 H) 2.65 (tq, J = 7.3, 3.8 Hz, 1 H) 3.26 (q, |
| | J = 7.3 Hz, 2 H) 4.50 (s, 2 H) 7.35 (td, J = 9.0, 2.2 Hz, 1 H) 7.50 (d, J = 0.7 |
| | Hz, 1 H) 7.90-7.98 (m, 2 H) 8.10 (br d, J = 3.7 Hz, 1 H) |
| Final Cpd 11 | ¹H NMR (400 MHz, DMSO-d₆) δ ppm 1.37 (d, J = 6.7 Hz, 6 H) 3.89 |
| | (spt, J = 6.6 Hz, 1 H) 4.85 (br s, 2 H) 7.12 (d, J = 0.9 Hz, 1 H) 7.42-7.50 |
| | (m, 1 H) 7.55 (s, 1 H) 7.53-7.60 (m, 1 H) 7.88 (d, J = 0.9 Hz, 1 H) 7.94 |
| | (d, J = 7.4 Hz, 1 H) 8.1 1 (d, J = 8.6 Hz, 1 H) 11.51 (br s, 1 H) |
| Final Cpd 12 | ¹H NMR (400 MHz, DMSO-d₆) δ ppm 1.38 (d, J = 6.5 Hz, 6 H) 3.84-3.95 |
| | (m, 1 H) 3.96 (s, 3 H) 4.89 (s, 2 H) 6.97 (dd, J = 7.7, 5.0 Hz, 1 H) |
| | 7.42-7.48 (m, 1 H) 7.49-7.60 (m, 2 H) 7.90 (dd, J = 5.0, 1.7 Hz, 1 H) |
| | 7.94 (d, J = 7.4 Hz, 1 H) 8.11 (d, J = 8.3 Hz, 1 H) 8.27 (dd, J = 7.6, 1.4 Hz, |
| | 1 H) 9.73 (s, 1 H) |
| Final Cpd 13 | ¹H NMR (400 MHz, DMSO-d₆) δ ppm 1.28-1.46 (m, 6 H) 2.38-2.43 |
| | (m, 3 H) 3.90 (spt, J = 6.7 Hz, 1 H) 4.82 (s, 2 H) 7.35 (dd, J = 5.5, 1.8 Hz, |
| | 1 H) 7.44 (d, J = 1.8 Hz, 1 H) 7.46 (t, J = 7.9 Hz, 1 H) 7.54-7.60 (m, 2 |
| | H) 7.95 (d, J = 7.4 Hz, 1 H) 8.11 (d, J = 8.3 Hz, 1 H) 8.30 (d, J = 5.5 Hz, 1 |
| | H) 10.51 (brs, 1 H) |
| Final Cpd 14 | ¹H NMR (400 MHz, DMSO-d₆) δ ppm 1.37 (d, J = 6.7 Hz, 6 H) 3.90 |
| | (spt, J = 6.7 Hz, 1 H) 4.91 (s, 2 H) 7.44-7.49 (m, 1 H) 7.55 (d, J = 0.7 |
| | Hz, 1 H) 7.55-7.60 (m, 1 H) 7.95 (d, J = 7.4 Hz, 1 H) 8.00 (dd, J = 5.8. |
| | 1.2 Hz, 1 H) 8.11 (d, J = 8.6 Hz, 1 H) 8.67 (d, J = 5.8 Hz, 1 H) 8.92 (d, |
| | J = 1.4 Hz, 1 H) 11.26 (s, 1 H) |
| Final Cpd 15 | ¹H NMR (400 MHz, DMSO-d₆) δ ppm 1.33 (t, J = 7.3 Hz, 3 H) 3.32 (q, |
| | J = 7.3 Hz, 2 H) 4.84 (s, 2 H) 7.34-7.38 (m, 1 H) 7.43-7.49 (m, 1 H) |
| | 7.51-7.58 (m, 2 H) 7.94 (d, J = 7.5 Hz, 1 H) 7.99-8.04 (m, 1 H) 8.13 |
| | (dd, J = 8.7, 0.6 Hz, 1 H) 8.29 (dd, J = 4.7, 14 Hz, 1 H) 8.74 (d, J = 2.2 |
| | Hz, 1 H) 10.42 (s, 1 H) |
| Final Cpd 16 | ¹H NMR (400 MHz, DMSO-d₆) δ ppm 0.36-0.49 (m, 2 H) 0.56-0.69 |
| | (m, 2 H) 1.32 (t, J = 7.3 Hz, 3 H) 2.65 (tq, J = 7.4, 3.9 Hz, 1 H) 3.29 (q, |
| | J = 7.3 Hz, 2 H) 4.51 (s, 2 H) 7.40-7.47 (m, 1 H) 7.48 (s, 1 H) 7.54 |
| | (ddd, J = 8.6, 7.2, 1.2 Hz, 1 H) 7.92 (d, J = 7.7 Hz, 1 H) 8.07-8.15 (m, 2 |
| | H) |
| Final Cpd 17 | ¹H NMR (400 MHz, DMSO-d₆) δ ppm 1.34 (t, J = 7.2 Hz, 3 w 3.32 (q, |
| | J = 7.5 Hz, 2 H) 3.86 (s, 3 H) 4.88 (s, 2 H) 6.90 (ddd, J = 8.1, 6.3, 2.3 Hz, |
| | 1 H) 7.04-7.12 (m, 2 H) 7.43-7.48 (m, 1 H) 7.52 (d, J = 0.4 Hz, 1 H) |
| | 7.52-7.57 (m, 1 H) 7.93 (br d, J = 7.9 Hz, 2 H) 8.10-8.16 (m, 1 H) |
| | 9.47 (s, 1 H) |
| Final Cpd 18 | ¹H NMR (400 MHz, DMSO-d₆) δ ppm 0.91-0.96 (m, 2 H) 1.05-1.12 |
| | (m, 2 H) 1.18-1.22 (m, 1 H) 4.87 (s, 2 H) 7.47 (ddd, J = 8.0, 7.1, 0.7 |
| | Hz, 1 H) 7.51 (d, J = 0.7 Hz, 1 H) 7.55 (ddd, J = 8.6. 7.1, 1.2 Hz, 1 H) |
| | 7.94 (br d, J = 7.6 Hz, 1 H) 8.00 (dd, J = 5.8, 1.2 Hz, 1 H) 8.48 (dd, |
| | J = 8.8, 0.7 Hz, 1 H) 8.67 (d, J = 5.8 Hz, 1 H) 8.91 (d, J = 0.9 Hz, 1 H) |
| | 11.24 (s, 1 H) |
| Final Cpd 19 | ¹H NMR (500 MHz, DMSO-d₆) δ ppm 2.79 (s, 6 H) 4.85 (s, 2 H) 7.42-7.47 |
| | (m, 1 H) 7.50 (d, J = 0.6 Hz, 1 H) 7.56 (ddd, J = 8.5, 7.2, 1.2 Hz, 1 |
| | H) 7.91 (brd, J = 7.9 Hz, 1 H) 8.01 (dd, J = 5.8, 1.1 Hz, 1 H) 8.15 (dd, |
| | J = 8.6, 0.7 Hz, 1 H) 8.67 (d, J = 5.8 Hz, 1 H) 8.91 (d, J = 1.2 Hz, 1 H) |
| | 11.24 (s, 1 H) |
| Final Cpd 20 | ¹H NMR (400 MHz, DMSO-d₆) δ ppm 1.32-1.54 (m, 2 H) 1.69 (br |
| | dd, J = 12.7, 2.3 Hz, 2 H) 2.78 (s, 6 H) 3.29-3.38 (m,2 H) 3.74-3.90 |
| | (m, 3 H) 4.50 (s, 2 H) 7.43 (ddd, J = 8.0, 7.1, 0.9 Hz, 1 H) 7.47 (d, J = 0.7 |
| | Hz, 1H) 7.54 (ddd, J = 8.5, 7.1, 1.3 Hz, 1 H) 7.86-7.93 (m, 1 H) 7.98 |
| | (d, J = 7.9 Hz, 1 H) 8.14 (dd, J = 8.6, 0.9 Hz, 1 H) |
| Final Cpd 21 | ¹H NMR (400 MHz, DMSO-d₆) δ ppm 1.09-1.31 (m, 4 H) 1.66-1.88 |
| | (m, 4 H) 2.78 (s, 6 H) 3.28-3.41 (m, 1 H) 3.46-3.58 (m, 1 H) 4.47 (s, |
| | 2 H) 4.51 (br s, 1 H) 7.43 (ddd, J = 8.0, 7.1, 1.0 Hz, 1 H) 7.46 (d, J = 0.7 |
| | Hz, 1 H) 7.53 (ddd, J = 8.5, 7.1, 1.3 Hz, 1 H) 7.82 (d, J = 7.9 Hz, 1 H) |
| | 7.89 (dt, J = 7.9, 1.2 Hz, 1 H) 8.14 (dd, J = 8.6, 0.9 Hz, 1 H) |
| Final Cpd 22 | ¹H NMR (500 MHz, DMSO-d₆) δ ppm 1.57 (d, J = 6.3 Hz, 3 H) 4.88-4.99 |
| | (m, 2 H) 5.37 (quin, J = 6.4 Hz, 1 H) 6.01 (d, J = 7.0 Hz, 1 H) 7.41-7.47 |
| | (m, 1 H) 7.50-7.57 (m, 2 H) 7.91 (d, J = 7.9 Hz, 1 H) 8.00 (d, J = 5.6 Hz, |
| | 1 H) 8.30 (d, J = 8.7 Hz, 1 H) 8.67 (d, J = 5.8 Hz, 1 H) 8.92 (s, 1 H) |
| | 11.29 (br s, 1 H) |
| Final Cpd 23 | ¹H NMR (400 MHz, DMSO-d₆) δ ppm 0.36-0.49 (m, 2 H) 0.55-0.69 |
| | (m, 2 H) 2.60-2.69 (m, 1 H) 2.87 (s, 3 H) 4.50 (s, 2 H) 7.42-7.47 (m, |
| | 1 H) 7.47 (d, J = 0.7 Hz, 1 H) 7.53 (ddd, J = 8.6, 7.2, 1.4 Hz, 1 H) 7.92 |
| | (d, J = 7.4 Hz, 1 H) 8.11 (br d, J = 3.9 Hz, 1 H) 8.15 (dd, J = 8.7, 0.8 Hz, 1 |
| | H) |
| Final Cpd 24 | ¹H NMR (400 MHz, DMSO-d₆) δ ppm 1.37-1.50 (m, 2 H) 1.63-1.75 |
| | (m, 2 H) 3.32 (s, 2 H) 3.76-3.88 (m, 3 H) 4.09 (s, 3 H) 4.50 (s, 2 H) |
| | 7.40-7.47 (m, 2 H) 7.53 (ddd, J = 8.5, 7.1, 1.3 Hz, 1H) 7.90 (dt, J = 7.9, |
| | 1.0 Hz, 1 H) 7.97 (d, J = 7.9 Hz, 1 H) 8.18 (dq, J = 8.4, 0.8 Hz, 1 H) |
| Final Cpd 25 | ¹H NMR (400 MHz, DMSO-d₆) δ ppm 1.38-1.53 (m, 2 H) 1.69-1.81 |
| | (m, 4 H) 1.98-2.09 (m, 2 H) 2.10-2.20 (m, 4 H) 3.27-3.41 (m, 2 H) |
| | 3.62 (quin, J = 9.1 Hz, 1 H) 3.77-3.88 (m, 3 H) 5.13 (s, 2 H) 7.42-7.51 |
| | (m, 2 H) 7.52 (s, 1 H) 7.89-7.93 (m, 1 H) 8.44 (d, J = 7.4 Hz, 1 H) |
| | 8.73-8.78 (m, 1 H) |
| Final Cpd 26 | ¹H NMR (400 MHz, DMSO-d₆) δ ppm 0.99 (d, J = 6.5 Hz, 6 H) 1.38-1.54 |
| | (m, 2 H) 1.76 (br dd, J = 12.5, 2.3 Hz, 2 H) 2.12-2.25 (m, 1 H) |
| | 3.08 (d, J = 7.6 Hz, 2 H) 3.33-3.43 (m, 2 H) 3.75-3.88 (m, 3 H) 4.96 |
| | (s, 2 H) 7.42-7.47 (m, 1 H) 7.47-7.52 (m, 1 H) 7.69 (s, 1 H) 7.90 (dd, |
| | J = 7.2, 0.9 Hz, 1 H) 8.45 (d, J = 7.4 Hz, 1 H) 8.73 (d, J = 8.3 Hz, 1 H) |
| Final Cpd 27 | ¹H NMR (400 MHz, DMSO-d₆) δ ppm 1.34-1.47 (m, 2 H) 1.74 (br |
| | dd, J = 12.5, 2.5 Hz, 2 H) 3.31-3.39 (m, 2 H) 3.75-3.86 (m, 3 H) 4.83 |
| | (s, 2 H) 7.12 (d, J = 0.5 Hz, 1 H) 7.36 (dd, J = 5.1, 3.5 Hz, 1 H) 7.44-7.49 |
| | (m, 1 H) 7.54 (ddd, J = 8.4, 7.1, 1.2 Hz, 1 H) 7.66 (dd, J = 3.6, 1.3 |
| | Hz, 1 H) 7.86-7.90 (m, 1 H) 8.09 (dd, J = 5.1, 1.4 Hz, 1 H) 8.38 (d, |
| | J = 7.6 Hz, 1 H) 8.76 (dq, J = 8.3, 0.9 Hz, 1 H) |
| Final Cpd 28 | ¹H NMR (500 MHz, DMSO-d₆) δ ppm 1.32-1.55 (m, 6 H) 1.70-1.80 |
| | (m, 3 H) 1.84 (br d, J = 13.0 Hz, 2 H) 1.92-2.02 (m, 2 H) 2.03-2.15 |
| | (m, 2 H) 3.27-3.44 (m, 2 H) 3.78-3.88 (m, 3 H) 5.13 (s, 2 H) 7.43-7.51 |
| | (m, 2 H) 7.85 (s, 1 H) 7.87-7.93 (m, 1 H) 8.41-8.52 (m, 1 H) |
| | 8.75 (d, J = 7.9 Hz, 1 H) |
| Final Cpd 29 | ¹H NMR (400 MHz, DMSO-d₆) δ ppm 0.40-0.44 (m, 2 H) 0.60-0.65 |
| | (m, 2 H) 1.36 (d, J = 6.5 Hz, 6 H) 2.60-2.66 (m, 1 H) 3.86 (quin, J = 6.6 |
| | Hz, 1 H) 4.50 (s, 2 H) 7.43-7.47 (m, 1 H) 7.51 (s, 1 H) 7.55 (ddd, |
| | J = 8.6, 7.1, 1.4 Hz, 1 H) 7.93 (d, J = 7.4 Hz, 1 H) 8.07-8.12 (m, 2 H) |
| Final Cpd 30 | ¹H NMR (400 MHz, DMSO-d₆) δ ppm 1.33 (t, J = 7.3 Hz, 3 H) 2.55 (s, |
| | 3H) 3.32 (q, J = 7.3 Hz, 2 H) 4.84 (s, 2 H) 7.36 (dd, J = 8.4, 4.6 Hz, 1 H) |
| | 7.42-7.50 (m, 1 H) 7.51-7.60 (m, 2 H) 7.94 (d, J = 7.5 Hz, 1 H) 7.98-8.06 |
| | (m, 1 H) 8.09-8.19 (m, 1 H) 8.29 (dd, J = 4.7, 1.4 Hz, 1 H) 8.74 |
| | (d, J = 2.2 Hz, 1 H) 10.42 (s, 1 H) |
| Final Cpd 31 | ¹H NMR (400 MHz, DMSO-d₆) δ ppm 1.30 (1, J = 7.2 Hz, 3 H) 3.26-3.29 |
| | (m, 2 H) 4.84 (s, 2 H) 7.37 (td, J = 9.1, 2.1 Hz, 1 H) 7.53-7.57 (m, 3 H) |
| | 7.92-8.00 (m, 2 H) 8.44 (d, J = 5.1 Hz, 2 H) 10.62 (s, 1 H) |
| Final Cpd 32 | ¹H NMR (400 MHz, DMSO-d₆) δ ppm 1.31 (1,J = 7.3 Hz, 3 H) 1.36-1.48 |
| | (m, 2 H) 1.67-1.73 (m, 2 H) 2.54 (s, 3 H) 3.25-3.38 (m, 4 H) |
| | 3.76-3.86 (m, 3 H) 4.54 (s, 2 H) 7.28 (d, J = 8.8 Hz, 1 H) 7.42 (s, 1 H) |
| | 7.80 (d, J = 8.1 Hz, 1 H) 7.91 (s, 1 H) 8.04 (d, J = 7.9 Hz, 1 H) |
| Final Cpd 33 | ¹H NMR (400 MHz, DMSO-d₆) δ ppm 1.26-1.39 (m, 3 H) 3.23-3.35 |
| | (m, 2 H) 4.92 (s, 2 H) 7.27-7.44 (m, 1 H) 7.51-7.60 (m, 1 H) 7.85-8.14 |
| | (m, 3 H) 8.66 (s, 1 H) 8.85-8.99 (m, 1 H) 11.20-11.41 (m, 1 H) |
| Final Cpd 34 | ¹H NMR (400 MHz, DMSO-d₆) δ ppm 1.31 (t, J = 7 2 Hz, 3 H) 1.37-1.48 |
| | (m, 2 H) 1.65-1.75 (m, 2 H) 2.47 (s, 3 H) 3.22-3.40 (m, 4 H) |
| | 3.76-3.85 (m, 3 H) 4.54 (s, 2 H) 7.36 (dd, J = 9.0, 1.5 Hz, 1 H) 7.39 (s, |
| | 1 H) 7.69 ( s, 1 H) 7.99 (d, J = 8.8 Hz, 1 H) 8.03 (br d, J = 7.7 Hz, 1 H) |
| Final Cpd 35 | ¹H NMR (400 MHz, DMSO-d₆) δ ppm 1.31 (t, J = 7.2 Hz, 3 H) 1.36-1.48 |
| | (m, 2 H) 1.67-1.74 (m, 2 H) 3.23-3.40 (m, 4 H) 3.75-3.87 (m, 3 H) |
| | 4.56 (s, 2 H) 7.39 (td, J = 9.2, 2.6 Hz, 1 H) 7.47 (s, 1 H) 7.71 (dd, |
| | J = 9.2, 2.6 Hz, 1 H) 8.04 (d, J = 7.7 Hz, 1 H) 8.15 (dd, J = 9.5, 4.4 Hz, 1 |
| | H) |
| Final Cpd 36 | ¹H NMR (400 MHz, DMSO-d₆) δ ppm 1.31 (t, J = 7.3 Hz, 3 H) 3.25-3.33 |
| | (m, 2 H) 4.93 (s, 2 H) 7.41 (td, J = 9.2, 2.6 Hz, 1 H) 7.50 (s, 1 H) |
| | 7.72 (dd, J = 9.0, 2.6 Hz, 1 H) 8.00 (dd, J = 5.7, 1.3 Hz, 1 H) 8.17 (dd, |
| | J = 9.5, 4.4 Hz, 1 H) 8.67 (d, J = 5.9 Hz, 1 H) 8.92 (d, J = 1.1 Hz, 1 H) |
| | 11.26 (s, 1 H) |
| Final Cpd 37 | ¹H NMR (400 MHz, DMSO-d₆) δ ppm 1.31 (t, J = 7.2 Hz, 3 H) 2.48 (s, |
| | 3 H) 3.24-3.34 (m, 2 H) 4.91 (s, 2 H) 7.38 (dd, J = 9.0, 1.5 Hz, 1 H) |
| | 7.43 (s, 1 H) 7.70 (s, 1 H) 7.98-8.03 (m, 2 H) 8.66 (d, J = 5.7 Hz, 1 H) |
| | 8.91 (d, J = 0.9 Hz, 1 H) 11.25 (s, 1 H) |
| Final Cpd 38 | ¹H NMR (400 MHz, CDCl₃) δ 1.43 (d, J = 6.6 Hz, 6H) 1.47 (t, J = 7.3 |
| | Hz, 3H) 3.28 (q, J = 7.3 Hz, 2H) 4.21-4.42 (m, 1H) 4.88 (s, 2H) 6.68 |
| | (d, J = 2.3 Hz, 1H) 7.30 (d, J = 2.3 Hz, 1H) 7.43 (t, J = 7.4 Hz, 1H) 7.51 |
| | (dd, J = 11.5, 4.2 Hz, 1H) 7.58 (s, 1H) 7.87 (d, J = 7.9 Hz, 1H) 7.93 (d, |
| | J = 8.7 Hz, 1H) 8.37 (s, 1H) |
| Final Cpd 39 | ¹H NMR (500 MHz, DMSO-d₆) δ ppm 1.21 (s, 3 H) 1.29 (t, J = 7.2 Hz, |
| | 3 H) 1.88-2.01 (m, 2 H) 2.16-2.28 (m, 2 H) 3.22-3.29 (m, 2 H) 3.71-3.85 |
| | (m, 1 H) 4.51 (s, 2 H) 4.97 (s, 1 H) 7.35 (td, J = 9.0, 2.2 Hz, 1 H) |
| | 7.50 (s, 1 H) 7.85-8.04 (m, 2 H) 8.23 (d, J = 7.0 Hz, 1 H) |
| Final Cpd 40 | ¹H NMR (400 MHz, DMSO-d₆) δ ppm 1.21 (s, 3 H) 1.88-2.03 (m, 2 |
| | H) 2.17-2.26 (m, 2 H) 2.77 (s, 6 H) 3.71-3.85 (m, 1 H) 4.45 (s, 2 H) |
| | 4.95 (br s, 1 H) 7.34 (td, J = 9.1,2.4 Hz, 1 H) 7.48 (s, 1 H) 7.88 (dd, |
| | J = 10.4, 2.3 Hz, 1 H) 7.94 (dd, J = 8.9, 5.7 Hz, 1 H) 8.17 (d, J = 7.4 Hz, 1 |
| | H) |
| Final Cpd 41 | ¹H NMR (500 MHz, DMSO-d₆) δ ppm 1.21 (s, 3 H) 1.35 (d, J = 6.6 Hz, |
| | 6 H) 1.89-2.00 (m, 2 H) 2.14-2.27 (m, 2 H) 3.74-3.91 (m, 2 H) 4.51 |
| | (s, 2 H) 5.00 (s, 1 H) 7.41-7.48 (m, 1 H) 7.51 (s, 1 H) 7.55 (ddd, |
| | J = 8.6, 7.2, 1.3 Hz, 1 H) 7.93 (d, J = 7.8 Hz, 1 H) 8.09 (d, J = 8.7 Hz, 1 H) |
| | 8.23 (d, J = 7.2 Hz, 1 H) |
| Final Cpd 42 | ¹H NMR (400 MHz, DMSO-d₆) δ ppm 1.21 (s, 3 H) 1.33 (d, J = 6.7 Hz, |
| | 6 H) 1.95 (td, J = 9.0, 2.1 Hz, 2 H) 2.17-2.29 (m, 2 H) 3.69-3.85 (m, 2 |
| | H) 4.50 (s, 2 H) 4.95 (s, 1 H) 7.35 (td, J = 9.1,2.0 Hz, 1 H) 7.52 (s, 1 H) |
| | 7.89 (dd, J = 11.1, 1.4 Hz, 1 H) 7.96 (dd, J = 8.8. 5. 8 Hz, 1 H) 8.20 (d, |
| | J = 7.2 Hz, 1 H) |
| Final Cpd 43 | ¹H NMR (500 MHz, DMSO-d₆) δ ppm 1.21 (s, 3 H) 1.31 (L J = 7.2 Hz, |
| | 3 H) 1.91-1.99 (m, 2 H) 2.16-2.28 (m, 2 H) 3.28 (q, J = 7.2 Hz, 2 H) |
| | 3.73-3.83 (m, 1 H) 4.52 (s, 2 H) 5.02 (s, 1 H) 7.42-7.47 (m, 1 H) |
| | 7.48 (s, 1 H) 7.53 (ddd, J = 8.6. 7.1,1.2 Hz, 1 H) 7.92 (d, J = 7.8 Hz, 1 H) |
| | 8.11 (d, J = 8.7 Hz, 1 H) 8.25 (d, J = 7.2 Hz, 1 H) |
| Final Cpd 44 | ¹H NMR (500 MHz, DMSO-d₆) δ ppm 1.21 (s, 3 H) 1.88-2.00 (m, 2 |
| | H) 2.14-2.27 (m, 2 H) 2.78 (s, 6 H) 3.79 (sxt, J = 8.0 Hz, 1 H) 4.46 (s, |
| | 2 H) 4.96 (s, 1 H) 7.38-7.48 (m, 2 H) 7.54 (ddd, J = 8.5, 7.2, 1.1 Hz, 1 |
| | H) 7.90 (d, J = 7.9 Hz, 1 H) 8.10-8.24 (m, 2 H) |
| Final Cpd 45 | ¹H NMR (500 MHz, DMSO-d₆) δ ppm 1.37 (d, J = 6.6 Hz, 6 H) 3.90 |
| | (spt, J = 6.6 Hz, 1 H) 4.85 (s, 2 H) 7.32 (dd, J = 9.8, 1.8 Hz, 1 H) |
| | 7.43-7.50 (m, 1 H) 7.54-7.60 (m, 2 H) 7.79 (d, J = 9.8 Hz, 1 H) 7.95 (d, |
| | J = 7.9 Hz, 1 H) 8.12 (d, J = 8.7 Hz, 1 H) 9.09-9.34 (m, 2 H) 10.49 (s, 1 |
| | H) |
| Final Cpd 46 | ¹H NMR (500 MHz, DMSO-d₆) δ ppm 1.35 (d, J = 6.6 Hz, 6 H) 3.85 |
| | (spt, J = 6.6 Hz, 1 H) 4.84 (s, 2 H) 7.32 (dd, J = 9.8, 1.8 Hz, 1 H) 7.39 (td, |
| | J = 9.0, 2.1 Hz, 1 H) 7.58 (s, 1 H) 7.79 (d, J = 9.8 Hz, 1 H) 7.93 (dd, |
| | J = 11.1, 1.7 Hz, 1 H) 7.99 (dd, J = 8.8, 5.9 Hz, 1 H) 9.21 (dd, J = 1.7. 0.9 |
| | Hz, 1 H) 9.25 (d, J = 0.6 Hz, 1 H) 10.49 (br s, 1 H) |
| Final Cpd 47 | ¹H NMR (400 MHz, DMSO-d₆) δ ppm 2.81 (s, 6 H) 4.80 (s, 2 H) 7.32 |
| | (dd, J = 9.7, 1.8 Hz, 1 H) 7.42-7.49 (m, 1 H) 7.52 (s, 1 H) 7.56 (ddd, |
| | J = 8.5, 7.2, 1.2 Hz, 1 H) 7.79 (d, J = 9.7 Hz, 1 H) 7.92 (d, J = 7.9 Hz, 1 H) |
| | 8.11-8.21 (m, 1 H) 9.21 (d, J = 0.7 Hz, 1 H) 9.23-9.28 (m, 1 H) 10.42 |
| | (s, 1 H) |
| Final Cpd 48 | ¹H NMR (400 MHz, DMSO-d₆) δ ppm 1.33 (t, J = 7.2 Hz, 3 H) 2.45-2.55 |
| | (m, 2 H) 4.86 (s, 2 H) 7.32 (dd, J = 9.7, 1.8 Hz, 1 H) 7.43-7.50 (m, |
| | 1 H) 7.51-7.59 (m, 2 H) 7.79 (d, J = 9.7 Hz, 1 H) 7.94 (d, J = 7.6 Hz, 1 |
| | H) 8.14 (d, J = 8.3 Hz, 1 H) 9.20 (dd, J = 1.8, 0.9 Hz, 1 H) 9.24 (d, J = 0.7 |
| | Hz, 1 H) 10.60 (br s, 1 H) |
| Final Cpd 49 | ¹H NMR (400 MHz, DMSO-d₆) δ ppm 1.38 (d, J = 6.6 Hz, 6 H) 3.90 |
| | (spt, J = 6.5 Hz, 1 H) 4.93 (s, 2 H) 7.47 (d, J = 7.5 Hz, 1 H) 7.52-7.62 |
| | (m, 2 H) 7.88-8.00 (m, 2 H) 8.04-8.19 (m, 1 H) 8.35 (dd, J = 10.1, 0.7 |
| | Hz, 1 H) 9.51 (d, J = 0.7 Hz, 1 H) 11.42 (br s, 1 H) |
| Final Cpd 50 | ¹H NMR (500 MHz, DMSO-d₆) δ ppm 0.38-0.45 (m, 2 H) 0.60-0.64 |
| | (m, 2 H) 2.61-2.69 (m, 1 H) 4.09 (s, 3 H) 4.45 (s, 2 H) 7.41-7.47 (m, |
| | 2 H) 7.50-7.55 (m, 1 H) 7.87-7.92 (m, 1 H) 8.03-8.08 (m, 1 H) 8.16-8.22 |
| | (m, 1 H) |

### Example B—Pharmaceutical Compositions

A compound of the invention (for instance, a compound of the examples) is brought into association with a pharmaceutically acceptable carrier, thereby providing a pharmaceutical composition comprising such active compound. A therapeutically effective amount of a compound of the invention (e.g. a compound of the examples) is intimately mixed with a pharmaceutically acceptable carrier, in a process for preparing a pharmaceutical composition.

### Example C—Biological Examples

The activity of a compound according to the present invention can be assessed by in vitro methods. A compound the invention exhibits valuable pharmacological properties, e.g. properties susceptible to inhibit NLRP3 activity, for instance as indicated the following test, and are therefore indicated for therapy related to NLRP3 inflammasome activity.

### PBMC Assay

Peripheral venous blood was collected from healthy individuals and human peripheral blood mononuclear cells (PBMCs) were isolated from blood by Ficoll-Histopaque (Sigma-Aldrich, A0561) density gradient centrifugation. After isolation, PBMCs were stored in liquid nitrogen for later use. Upon thawing, PBMC cell viability was determined in growth medium (RPMI media supplemented with 10% fetal bovine serum, 1% Pen-Strep and 1% L-glutamine). Compounds were spotted in a 1:3 serial dilution in DMSO and diluted to the final concentration in 30 μl medium in 96 well plates (Falcon, 353072). PBMCs were added at a density of 7.5×104 cells per well and incubated for 30 min in a 5% C02 incubator at 37° C. LPS stimulation was performed by addition of 100 ng/ml LPS (final concentration, Invivogen, tlrl-smlps) for 6 hrs followed by collection of cellular supernatant and the analysis of IL-1β (μM) and TNF cytokines levels (μM) via MSD technology according to manufacturers' guidelines (MSD, K151A0H).

The IC₅₀ values (for IL-1β) and EC₅₀ values (TNF) were obtained on compounds of the invention/examples, and are depicted in the following table:

| | | IL1β IC₅₀ | TNF EC₅₀ |
|---|---|---|---|
| Number | Compound | (μM) | (μM) |
| Final Cpd 1 | 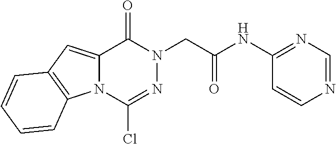 [SMILES: O=C(Cn1nc(Cl)n2c(cc3ccccc32)c1=O)Nc1ccncn1] | 0.62 | >10 |
| Final Cpd 2 | 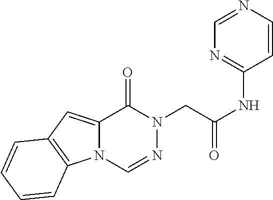 [SMILES: O=C(Cn1ncn2c(cc3ccccc32)c1=O)Nc1ccncn1] | 1.89 | >10 |
| Final Cpd 3 | 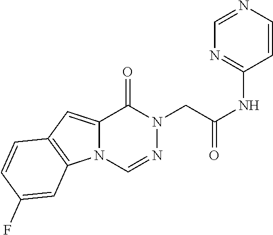 [SMILES: O=C(Cn1ncn2c(cc3ccc(F)cc32)c1=O)Nc1ccncn1] | 1.58 | >10 |
| Final Cpd 4 | 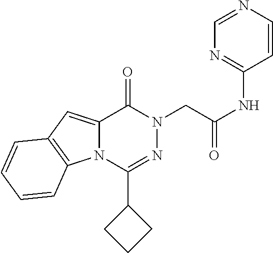 [SMILES: O=C(Cn1nc(C2CCC2)n2c(cc3ccccc32)c1=O)Nc1ccncn1] | 1.54 | >10 |
| Final Cpd 5 | 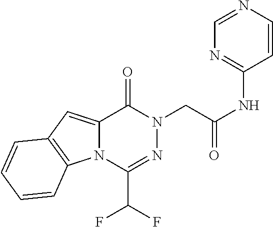 [SMILES: O=C(Cn1nc(C(F)F)n2c(cc3ccccc32)c1=O)Nc1ccncn1] | 0.47 | >10 |
| Final Cpd 6 | 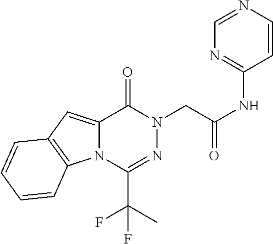 [SMILES: CC(F)(F)c1nn(CC(=O)Nc2ccncn2)c(=O)c2cc3ccccc3n12] | >10 | >10 |
| Final Cpd 7 | 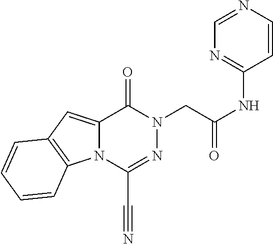 [SMILES: N#Cc1nn(CC(=O)Nc2ccncn2)c(=O)c2cc3ccccc3n12] | 3.50 | >10 |
| Final Cpd 8 | 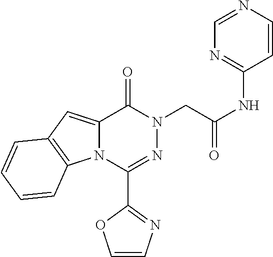 [SMILES: O=C(Cn1nc(-c2ncco2)n2c(cc3ccccc32)c1=O)Nc1ccncn1] | 5.80 | >10 |
| Final Cpd 9 | 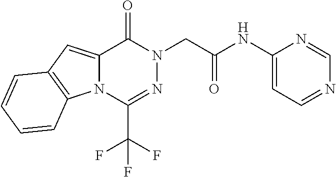 [SMILES: O=C(Cn1nc(C(F)(F)F)n2c(cc3ccccc32)c1=O)Nc1ccncn1] | 1.91 | >10 |
| Final Cpd 10 | 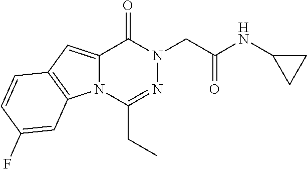 [SMILES: CCc1nn(CC(=O)NC2CC2)c(=O)c2cc3ccc(F)cc3n12] | 1.68 | >10 |
| Final Cpd 11 | 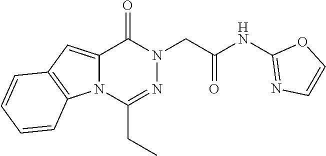 [SMILES: CCc1nn(CC(=O)Nc2ncco2)c(=O)c2cc3ccccc3n12] | 0.84 | >10 |
| Final Cpd 12 | 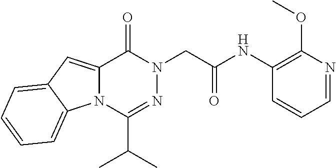 [SMILES: COc1ncccc1NC(=O)Cn1nc(C(C)C)n2c(cc3ccccc32)c1=O] | 0.41 | >10 |
| Final Cpd 13 | 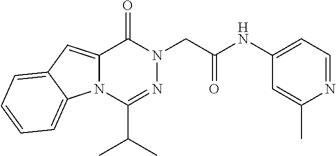 [SMILES: Cc1cc(NC(=O)Cn2nc(C(C)C)n3c(cc4ccccc43)c2=O)ccn1] | 0.15 | >10 |
| Final Cpd 14 | 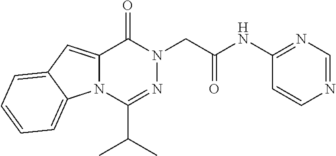 [SMILES: CC(C)c1nn(CC(=O)Nc2ccncn2)c(=O)c2cc3ccccc3n12] | 0.17 | >10 |
| Final Cpd 15 | 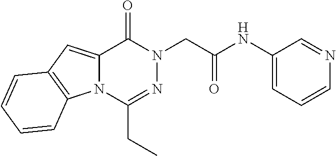 [SMILES: CCc1nn(CC(=O)Nc2cccnc2)c(=O)c2cc3ccccc3n12] | 0.63 | >10 |
| Final Cpd 16 | 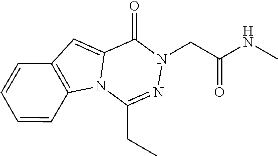 [SMILES: CCc1nn(CC(=O)NC)c(=O)c2cc3ccccc3n12] | 0.66 | >10 |
| Final Cpd 17 | 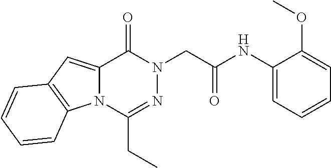 [SMILES: CCc1nn(CC(=O)Nc2ccccc2OC)c(=O)c2cc3ccccc3n12] | 2.80 | >10 |
| Final Cpd 18 | 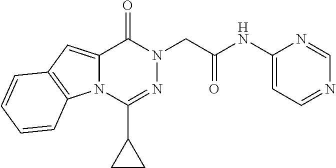 [SMILES: O=C(Cn1nc(C2CC2)n2c(cc3ccccc32)c1=O)Nc1ccncn1] | 0.76 | >10 |
| Final Cpd 19 | 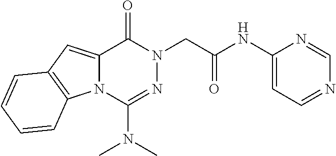 [SMILES: CN(C)c1nn(CC(=O)Nc2ccncn2)c(=O)c2cc3ccccc3n12] | | |
| Final Cpd 20 | 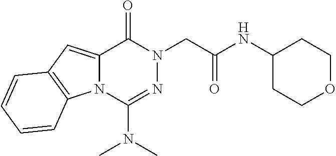 [SMILES: CN(C)c1nn(CC(=O)NC2CCOCC2)c(=O)c2cc3ccccc3n12] | 0.31 | >10 |
| Final Cpd 21 | 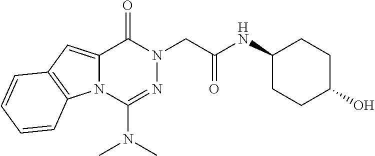 [SMILES: CN(C)c1nn(CC(=O)N[C@H]2CC[C@H](O)CC2)c(=O)c2cc3ccccc3n12] | 0.39 | >10 |
| Final Cpd 22 | 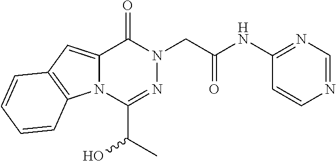 [SMILES: CC(O)c1nn(CC(=O)Nc2ccncn2)c(=O)c2cc3ccccc3n12] | 1.44 | >10 |
| Final Cpd 23 | 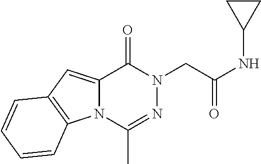 [SMILES: Cc1nn(CC(=O)NC2CC2)c(=O)c2cc3ccccc3n12] | 9.12 | >10 |
| Final Cpd 24 | 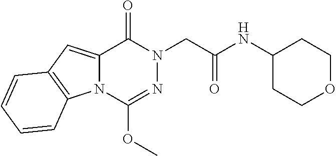 [SMILES: COc1nn(CC(=O)NC2CCOCC2)c(=O)c2cc3ccccc3n12] | 1.17 | >10 |
| Final Cpd 25 | 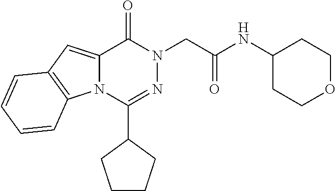 [SMILES: O=C(Cn1nc(C2CCCC2)n2c(cc3ccccc32)c1=O)NC1CCOCC1] | >10 | >20 |
| Final Cpd 26 | 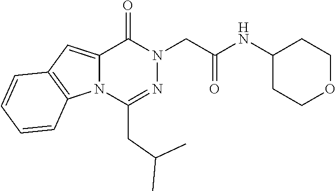 [SMILES: CC(C)Cc1nn(CC(=O)NC2CCOCC2)c(=O)c2cc3ccccc3n12] | >10 | >10 |
| Final Cpd 27 | 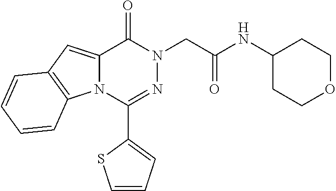 [SMILES: O=C(Cn1nc(-c2cccs2)n2c(cc3ccccc32)c1=O)NC1CCOCC1] | >10 | >10 |
| Final Cpd 28 | 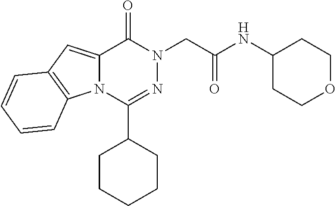 [SMILES: O=C(Cn1nc(C2CCCCC2)n2c(cc3ccccc32)c1=O)NC1CCOCC1] | >10 | >10 |
| Final Cpd 29 | 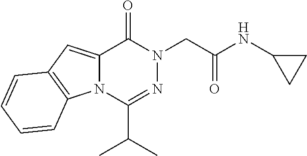 [SMILES: CC(C)c1nn(CC(=O)NC2CC2)c(=O)c2cc3ccccc3n12] | 0.66 | >10 |
| Final Cpd 30 | 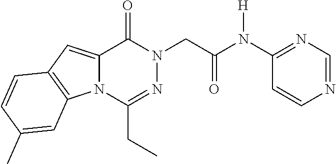 [SMILES: CCc1nn(CC(=O)Nc2ccncn2)c(=O)c2cc3ccc(C)cc3n12] | >10 | >10 |
| Final Cpd 31 | 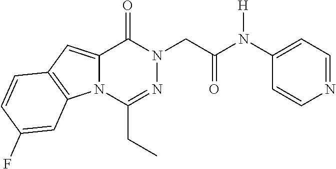 [SMILES: CCc1nn(CC(=O)Nc2ccncc2)c(=O)c2cc3ccc(F)cc3n12] | 0.79 | >10 |
| Final Cpd 32 | 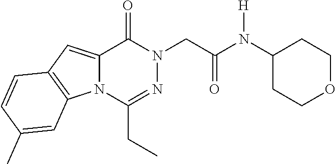 [SMILES: CCc1nn(CC(=O)NC2CCOCC2)c(=O)c2cc3ccc(C)cc3n12] | >10 | >10 |
| Final Cpd 33 | 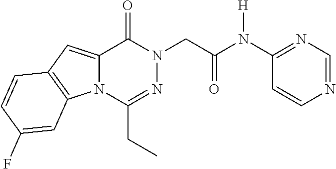 [SMILES: CCc1nn(CC(=O)Nc2ccncn2)c(=O)c2cc3ccc(F)cc3n12] | 0.31 | >10 |
| Final Cpd 34 | 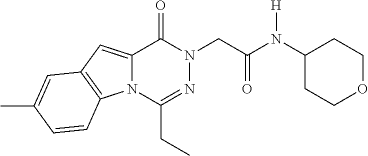 [SMILES: CCc1nn(CC(=O)NC2CCOCC2)c(=O)c2cc3cc(C)ccc3n12] | 7.24 | >10 |
| Final Cpd 35 | 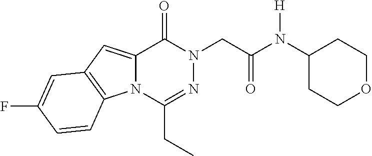 [SMILES: CCc1nn(CC(=O)NC2CCOCC2)c(=O)c2cc3cc(F)ccc3n12] | >10 | >10 |
| Final Cpd 36 | 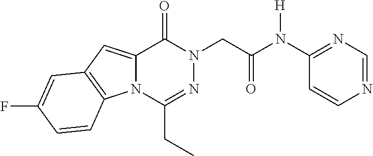 [SMILES: CCc1nn(CC(=O)Nc2ccncn2)c(=O)c2cc3cc(F)ccc3n12] | 7.24 | >10 |
| Final Cpd 37 | 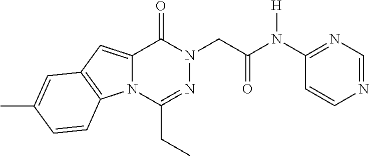 [SMILES: CCc1nn(CC(=O)Nc2ccncn2)c(=O)c2cc3cc(C)ccc3n12] | >10 | >10 |
| Final Cpd 38 | 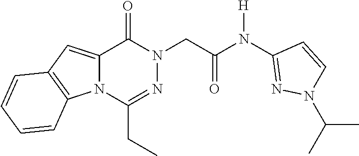 [SMILES: CCc1nn(CC(=O)Nc2ccn(C(C)C)n2)c(=O)c2cc3ccccc3n12] | 7.7 | >20 |
| Final Cpd 39 | 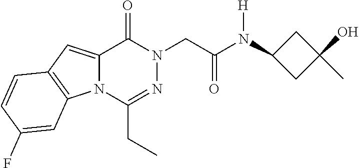 [SMILES: CCc1nn(CC(=O)N[C@H]2C[C@@](C)(O)C2)c(=O)c2cc3ccc(F)cc3n12] | 0.176 | >20 |
| Final Cpd 40 | 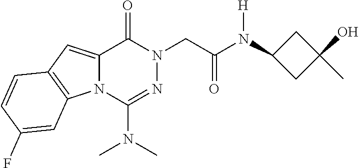 [SMILES: CN(C)c1nn(CC(=O)N[C@H]2C[C@@](C)(O)C2)c(=O)c2cc3ccc(F)cc3n12] | 0.237 | >20 |
| Final Cpd 41 | 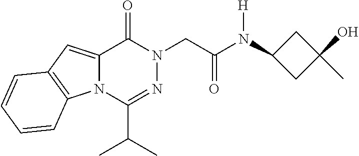 [SMILES: CC(C)c1nn(CC(=O)N[C@H]2C[C@@](C)(O)C2)c(=O)c2cc3ccccc3n12] | 0.052 | 15.4 |
| Final Cpd 42 | 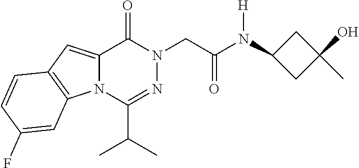 [SMILES: CC(C)c1nn(CC(=O)N[C@H]2C[C@@](C)(O)C2)c(=O)c2cc3ccc(F)cc3n12] | 0.077 | 10.9 |
| Final Cpd 43 | 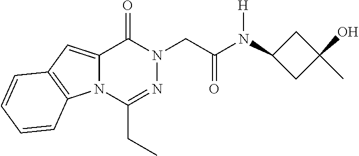 [SMILES: CCc1nn(CC(=O)N[C@H]2C[C@@](C)(O)C2)c(=O)c2cc3ccccc3n12] | 0.086 | >20 |
| Final Cpd 44 | 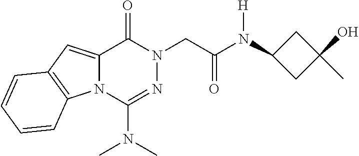 [SMILES: CN(C)c1nn(CC(=O)N[C@H]2C[C@@](C)(O)C2)c(=O)c2cc3ccccc3n12] | 0.057 | >20 |
| Final Cpd 45 | 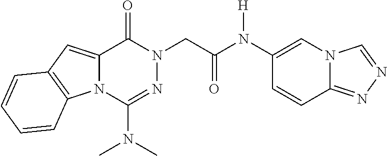 [SMILES: CN(C)c1nn(CC(=O)Nc2ccc3nncn3c2)c(=O)c2cc3ccccc3n12] | 0.024 | >20 |
| Final Cpd 46 | 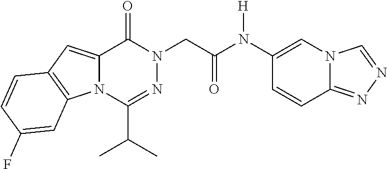 [SMILES: CC(C)c1nn(CC(=O)Nc2ccc3nncn3c2)c(=O)c2cc3ccc(F)cc3n12] | 0.044 | >20 |
| Final Cpd 47 | 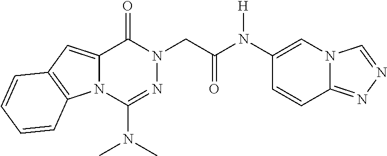 [SMILES: CN(C)c1nn(CC(=O)Nc2ccc3nncn3c2)c(=O)c2cc3ccccc3n12] | 0.052 | >20 |
| Final Cpd 48 | 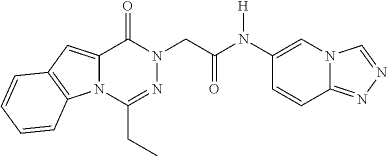 [SMILES: CCc1nn(CC(=O)Nc2ccc3nncn3c2)c(=O)c2cc3ccccc3n12] | 0.122 | >20 |
| Final Cpd 49 | 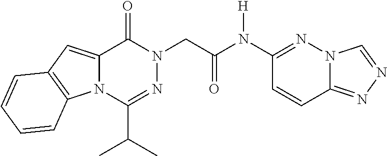 [SMILES: CC(C)c1nn(CC(=O)Nc2ccc3nncn3n2)c(=O)c2cc3ccccc3n12] | 0.025 | 3.94 |
| Final Cpd 50 | 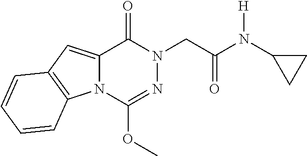 [SMILES: COc1nn(CC(=O)NC2CC2)c(=O)c2cc3ccccc3n12] | 4.07 | >10 |

### Example D—Further Testing

One or more compound(s) of the invention (including compounds of the final examples) is/are tested in a number of other methods to evaluate, amongst other properties, permeability, stability (including metabolic stability and blood stability) and solubility.

### Permeability Test

The in vitro passive permeability and the ability to be a transported substrate of P-glycoprotein (P-gp) is tested using MDCK cells stably transduced with MDR1 (this may be performed at a commercial organisation offering ADME, PK services, e.g. Cyprotex). Permeability experiments are conducted in duplicate at a single concentration (5 μM) in a transwell system with an incubation of 120 min. The apical to basolateral (AtoB) transport in the presence and absence of the P-gp inhibitor GF120918 and the basolateral to apical (BtoA) transport in the absence of the P-gp inhibitor is measured and permeation rates (Apparent Permeability) of the test compounds (Pₐₚₚ×10⁻⁶ cm/sec) are calculated.

### Metabolic Stability Test in Liver Microsomes

The metabolic stability of a test compound is tested (this may be performed at a commercial organisaiton offering ADME, PK services, e.g. Cyprotex) by using liver microsomes (0.5 mg/ml protein) from human and preclinical species incubated up to 60 minutes at 37° C. with 1 μM test compound.

The in vitro metabolic half-life (t_{1/2}) is calculated using the slope of the log-linear regression from the percentage parent compound remaining versus time relationship (κ),

t_{1/2}=−ln(2)/κ.

The in vitro intrinsic clearance (Clᵢₙₜ) (ml/min/mg microsomal protein) is calculated using the following formula:

$${Cl}_{int} = \frac{0.693}{{t}_{1 / 2}} ⨯ \frac{{V}_{inc}}{{W}_{mic prot , inc}}$$

Where: V_{inc}=incubation volume,
W_{mic prot,inc}=weight of microsomal protein in the incubation.

### Metabolic Stabity Test in Uver Hepatocytes

The metabolic stability of a test compound is tested using liver hepatocytes (1 milj cells) from human and preclinical species incubated up to 120 minutes at 37° C. with 1 μM test compound.

The in vitro metabolic half-life (tin) is calculated using the slope of the log-linear regression from the percentage parent compound remaining versus time relationship (c), t_{1/2}=−ln(2)/κ.

The in vitro intrinsic clearance (Clᵢₙₜ) (μl/min/million cells) is calculated using the following formula:

$${C l}_{int} = \frac{0.693}{{t}_{1 / 2}} ⨯ \frac{{V}_{inc}}{\# {cells}_{inc}} x 1000$$

Where: V_{inc}=incubation volume,
# cells_{inc}=number of cells (×10⁶) in the incubation

### Solubillty Test

The test/assay is run in triplicate and is semi-automated using the Tecan Fluent for all liquid handling with the following general steps:
20 μl of 10 mM stock solution is dispensed in a 500 μl 96 well plate
DMSO is evaporated (Genevac)
a stir bar and 400 μl of buffer/biorelevant media is added
the solution is stirred for 72 h (pH2 and pH7) or 24 h (FaSSIF and FeSSIF)
the solution is filtered
the filtrate is quantified by UPLC/UV using a three-points calibration curve
   The LC conditions are:
Waters Acquity UPLC
Mobile phase A: 0.1% formic acid in H2O, B: 0.1% formic acid in CH3CN
Column: Waters HSS T3 1.8 μm 2.1×50 mm
Column temp.: 55° C.
Inj. vol.: 2 μl
Flow: 0.6 ml/min
Wavelength UV: 250_350 nm
Gradient: 0 min: 0% B, 0.3 min: 5% B, 1.8 min: 95% B, 2.6 min: 95% B

### Blood Stability Assay

The compound of the invention/examples is spiked at a certain concentration in plasma or blood from the agreed preclinical species; then after incubating to predetermined times and conditions (37° C., 0° C. (ice) or room temperature) the concentration of the test compound in the blood or plasma matrix with LCMS/MS can then be determined.

1. A compound of formula (I), 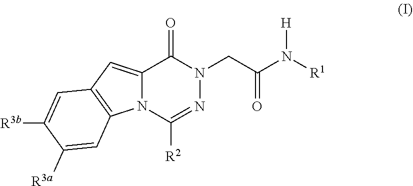
or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
R² represents:
(i) hydrogen;
(ii) halo;
(iii) —CN;
(iv) C₁₋₆ alkyl optionally substituted with one or more substituents independently selected from halo, —OH, —OC₁₋₃alkyl and oxo;
(v) C₃₋₆ cycloalkyl;
(vi) C₂₋₄alkenyl optionally substituted with —OC₁₋₃alkyl;
(vii) —O—C₁₋₃alkyl;
(viii) —N(R^{2a})R^{2b}; or
(ix) 5-membered heteroaryl, optionally substituted by one or more substituents selected from halo, C₁₋₃ alkyl and —OC₁₋₃ alkyl;
each R^{2a} and R^{2b} independently represent hydrogen or C₁₋₄alkyl optionally substituted with —OC₁₋₃ alkyl;
either one of R^{3a} and R^{3b} represents hydrogen and the other represents R³;
R³ represents:
(i) hydrogen;
(ii) halo; or
(iii) C₁₋₃ alkyl
but wherein:
(i) when R² represents hydrogen, R^{3a} and R^{3b} both represent hydrogen, then R¹ does not represent 2,3,4-trimethoxyphenyl, 2,4-dimethylcyclohexyl, 2-ethylphenyl, 3,4-dimethoxyphenyl, 3,4-dimethylphenyl, 3,5-dimethylphenyl, 3-ethylphenyl, 3-fluorophenyl, 4-ethylphenyl, 4-isopropylphenyl (or 4-propan-2-yl-phenyl) or cyclopropyl;
(ii) when R^{3a} represents hydrogen, R^{3b} represents hydrogen and R^{3b} represents fluoro, then R¹ does not represent 1,2,3,4-tetrahydronaphthalen-1-yl, 1(R)-1,2,3,4-tetrahydronaphthalen-1-yl, cyclohexyl or cyclopropyl;
(iii) when R² represents methyl, R^{3a} represents hydrogen and R^{3b} represents fluoro, then R¹ does not represent 1(S),2(R)-2-methylcyclohexyl, 2-methylcyclohexyl, 2,3-dimethylcyclohexyl, (1R),(2R),3(R)-2,3-dimethylcyclohexyl, (1R),(2R),3(S)-2,3-dimethylcyclohexyl, (1R),(2S),3(R)-2,3-dimethylcyclohexyl, (1R),(2S),3(S)-2,3-dimethylcyclohexyl, cyclohexyl or cyclopropyl;
(iv) when R² represents methyl or ethyl, R^{3a} and R^{3b} both represent hydrogen, then R¹ does not represent cyclopropyl.

2. The compound of claim 1, wherein R³ represents hydrogen or halo.

3. The compound of claim 1, wherein:
at least one of R^{3a} and R^{3b} does not represent hydrogen; and/or
R² does not represent hydrogen, methyl or ethyl.

4. The compound of claim 1, wherein R¹ represents C₃₋₆ cycloalkyl optionally substituted by one or two substituents selected from C₁₋₃ alkyl and —OH.

5. The compound of claim 4, wherein R¹ represents: 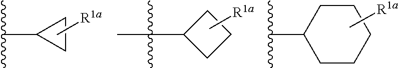
where each R^{1a} represents one or two optional substituents selected from —OH and C₁₋₃ alkyl.

6. The compound of claim 1, wherein R¹ represents a mono-cyclic 5- or 6-membered heterocyclyl group containing at least one nitrogen or oxygen heteroatom, and which is optionally substituted by one substituent selected from C₁₋₃ alkyl and C₃₋₆ cycloalkyl.

7. The compound of claim 1, wherein R¹ represents: (i) phenyl; (ii) a 5- or 6-membered mono-cyclic heteroaryl group; or (iii) a 9- or 10-membered bicyclic heteroaryl group, all of which are optionally substituted with one or two substituent(s) selected from halo, —OH, C₁₋₃ alkyl and —OC₁₋₃ alkyl.

8. The compound of claim 7, wherein R¹ represents phenyl or a mono-cyclic 6-membered heteroaryl group: 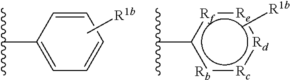
wherein R^{1b} represents one or two optional substituents selected from halo, —CH₃, —OH and —OCH₃, and, either one or two of R_{b}, R_{c}, R_{d}, Rₑ and R_{f} represent(s) a nitrogen heteroatom (and the others represent a CH).

9. The compound of claim 7, wherein R¹ represents: 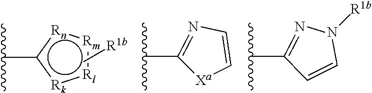
wherein R^{1b} is as defined in claim 8, and at least one of Rₖ, Rₗ, Rₘ and Rₙ represents a nitrogen heteroatom, and the others are independently selected from CH, N, O and S.

10. The compound of claim 7, wherein R¹ represents a monocyclic 5-membered heteroaryl group: 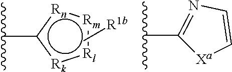
wherein R^{1b} is as defined in claim 8, one of Rₖ and Rₙ represents N, the other represents N, O, S or CH, and Rₗ and Rₘ each represent CH, and, X^{a} represents N, O, S or CH.

11. The compound of claim 7, wherein R¹ represents a 9- or 10-membered bicyclic heteroaryl group, for instance: 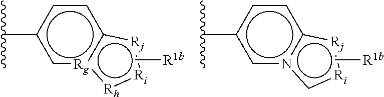
wherein R^{1b} represents one or two optional substituent selected from halo, —OH and —OCH₃, each ring of the bicyclic system is aromatic, R_{g} represents a N or C atom and any one or two of Rₕ, Rᵢ and Rⱼ represents N and the other(s) represent(s) C.

12. The compound of claim 7, wherein R¹ represents: 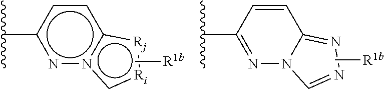
in which any one or two of Rᵢ and Rⱼ represents N and the other, if applicable, represents CH and R^{1b} represents one or more optional substituents as defined in claim 8 or claim 11).

13. The compound of claim 1, wherein R² represents: (i) hydrogen; (ii) halo; (iii) —CN; (iv) C₁₋₄ alkyl optionally substituted with one or more substituents independently selected from halo, —OH and —OC₁₋₂ alkyl; (v) C₃₋₆ cycloalkyl;
(vi) —O—C₁₋₂alkyl; (vii) —N(R^{2a})R^{2b}; or (viii) 5-membered heteroaryl.

14. The compound of claim 1, wherein each R^{2a} and R^{2b} independently represent hydrogen or unsubstituted C₁₋₄ alkyl.

15. The compound of claim 1, wherein R^{2a} and R^{2b} represents C₁₋₃ alkyl.

16. The compound of claim 1, wherein R³ represents (i) hydrogen; or (ii) fluoro.

17. The compound of claim 1, wherein one of R^{3a} and R^{3b} represents hydrogen and other represents hydrogen or fluoro.

18. A pharmaceutical composition comprising a therapeutically effective amount of a compound as defined in claim 1 but without the provisos and a pharmaceutically acceptable carrier.

19. (canceled)

20. (canceled)

21. A combination comprising: (a) a compound according to claim 1 but without the provisos; and (b) one or more other therapeutic agents.

22. (canceled)

23. A method of treating a disease or disorder associated with inhibition of NLRP3 inflammasome activity in a subject in need thereof, the method comprising administering to said subject a therapeutically effective amount of a compound according to claim 1 but without the provisos.

24. The method of treating according to claim 23 wherein the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

25. A process for the preparation of a compound of formula (I) as claimed in claim 1, which comprises:
(i) reaction of a compound of formula (II), 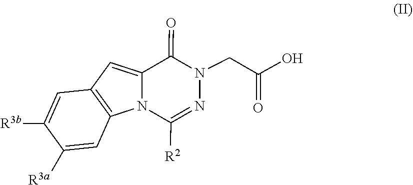
or a derivative thereof, wherein R², R^{3a} and R^{3b} are as defined in claim 1, with a compound of formula (III),
H₂N—R¹ (III)
or a derivative thereof, wherein R¹ is as defined in claim 1, under amide-forming reaction conditions;
(ii) reaction of a compound of formula (IV), 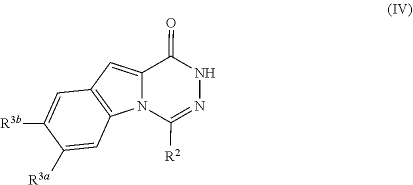
wherein R², R^{3a} and R^{3b} are as defined in claim 1, with a compound of formula (V),
LG^{a}-CH₂—C(O)—N(H)R¹ (V)
wherein LG^{a} represents a suitable leaving group and R¹ is as defined in claim 1;
(iii) by transformation of a certain compound of formula (I) into another.

26. A compound of formula (II) or a compound of formula (IV): 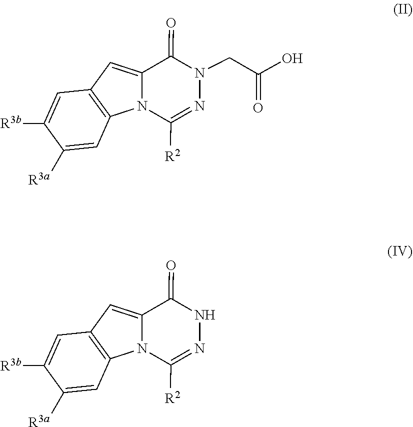
wherein R², R^{3a} and R^{3b} are as defined in claim 1.
